(19) United States
(12) Patent Application Publication
(10) Pub. No.: US 2026/0176272 A1
(43) Pub. Date: Jun. 25, 2026

(54) CATALYSTS AND METHODS OF USE THEREOF

(71) Applicant: Yale University, New Haven, CT (US)

(72) Inventors: Mingjiang Zhong, New Haven, CT (US); Xiaowei Zhang, New Haven, CT (US)

(21) Appl. No.: 19/129,236

(22) PCT Filed: Nov. 21, 2023
(86) PCT No.: PCT/US2023/080673

§ 371 Date: May 12, 2025

Related U.S. Application Data
(60) Provisional application No. 63/427,662, filed on Nov. 23, 2022.

(51) Int. Cl. C07D 471/10; C07D 487/04

(52) U.S. Cl. CPC C07D 471/10; C07D 487/04

(57) ABSTRACT

The present disclosure relates, in part, to catalyst compounds and methods of use thereof for stereocontrolled living radical polymerization. In another aspect, the present disclosure relates to isotactic polymers and/or polymers having controlled tacticity, and applications thereof. In certain embodiments, the isotactic polymers and/or controlled tacticity polymers are prepared according to the methods of the present disclosure.

### CROSS-REFERENCE TO RELATED APPLICATIONS

This application claims priority under 35 U.S.C. § 119(e) to U.S. Provisional Patent Application No. 63/427,662, filed Nov. 23, 2022, which is incorporated herein by reference in its entirety.

### STATEMENT REGARDING FEDERALLY SPONSORED RESEARCH OR DEVELOPMENT

This invention was made with government support under CHE-2108681 awarded by the National Science Foundation. The government has certain rights in the invention.

### BACKGROUND

The material properties of polymers with a given chemical composition can be tuned through engineering the relative stereochemistry of repeat units, also known as tacticity.

Vinyl polymers with varied backbone stereoregularities exhibit different thermomechanical properties, hole/charge carrier mobility, dielectric properties, self-assembly behaviors, processability, and permeation properties.

These intrinsic structure-property relationships motivated the development of stereocontrolled vinyl polymerizations to access polymer products with diversified properties from unchanged monomer feedstocks. However, stereocontrolled vinyl polymerizations have been limited to a small group of monomers and demand rigorously developed reaction conditions.

Coordination polymerization, which uses organometallic chain ends to perform stereospecific monomer insertion, predominates the industrial production of stereoregular poly(α-olefins). Polymers with enolizable propagating chain ends, such as polymethacrylates and poly acrylamides, can be synthesized in a stereoregular manner through a coordination-addition mechanism. Stereocontrolled ionic polymerizations were developed by introducing propagating chain ends with predetermined stereochemistry or countered with chiral ionic auxiliaries.

Radical polymerization (RP) holds several advantageous aspects compared to coordination and ionic polymerization methods, including its compatibility with significantly expanded libraries of vinyl monomer structures and pendant functionalities, relatively high tolerance to impurities, and ability to be conducted in aqueous and other protic reaction media.

However, development of stereocontrolled RP remains a fundamental challenge primarily due to the difficulty in controlling the stereochemistry of the radical propagating chain ends with an sp²-hybridized planar geometry. The continuous generation of new stereogenic radical center during the chain-growth polymerization process further complicates the development of stereocontrolled RP.

There is thus a need in the art for catalysts suitable for stereocontrolled radical polymerization, and methods of use thereof. The present disclosure addresses this need.

### BRIEF SUMMARY

In one aspect, the present disclosure provides a compound of Formula (Ia), or a salt, solvate, stereoisomer, or isotopologue thereof, wherein M, Z, R¹, R^{2a}, R^{2b}, R^{2c}, R^{3a}, R^{3b}, R^{3c}, R^{3d}, R^{3e}, R^{3f}, R^{3g}, and R^{3h} are defined elsewhere herein:

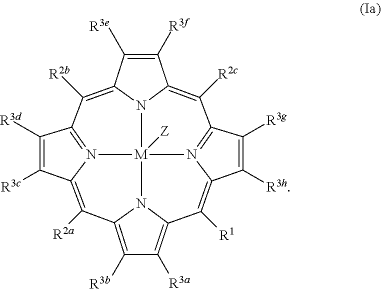

In one aspect, the present disclosure provides a compound of Formula (Ib), or a salt, solvate, stereoisomer, or isotopologue thereof, wherein M, Z, R¹, R^{3a}, R^{3b}, R^{3c}, R^{3d}, R^{3e}, R^{3f}, and R^{3g} are defined elsewhere herein:

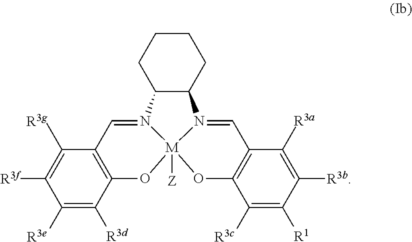

In one aspect, the present disclosure provides a compound of Formula (Ic), or a salt, solvate, stereoisomer, or isotopologue thereof, wherein R¹, R^{2a}, R^{3a}, R^{3b}, R^{3c}, and R^{3d} are defined elsewhere herein:

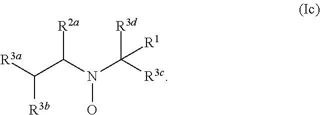

In one aspect, the present disclosure provides a compound of Formula (Id), or a salt, solvate, stereoisomer, or isotopologue thereof, wherein R¹, R^{3a}, R^{3b}, R^{3c}, and R^{3d} are defined elsewhere herein:

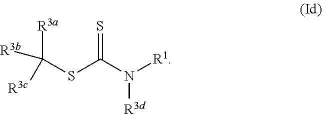

In one aspect, the present disclosure provides a compound of Formula (Ie), or a salt, solvate, stereoisomer, or isotopologue thereof, wherein R^{3a}, R^{3b}, R^{3c}, and R⁴ are defined elsewhere herein:

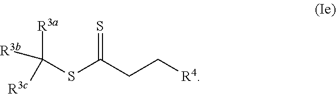

In one aspect, the present disclosure provides a compound of Formula (If), or a salt, solvate, stereoisomer, or isotopologue thereof, wherein R^{3a}, R^{3b}, R^{3c}, and R⁴ are defined elsewhere herein:

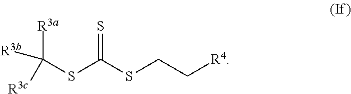

In one aspect, the present disclosure provides a compound of Formula (Ig), or a salt, solvate, stereoisomer, or isotopologue thereof, wherein R^{3a}, R^{3b}, R^{3c}, and R⁴ are defined elsewhere herein:

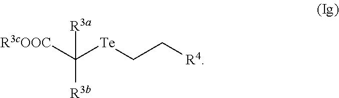

In another aspect, the present disclosure provides a polymer composition of Formula (III):

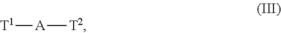

wherein:
each occurrence of T¹ is independently selected from the group consisting of C(═O)OR¹³, optionally substituted alkyl, and halogen;
T² is selected from the group consisting of H,

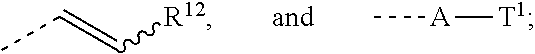

each occurrence of A independently comprises o units of

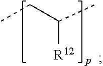

each occurrence o is independently an integer ranging from 1 to 10;
each occurrence of p is independently an integer ranging from 1 to 5,000;
R¹² is substituted with at least one substituent comprising a heteroatom having at least one coordinating lone pair of electrons,
   wherein the R¹² groups in each unit of

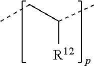

are identical; and
each occurrence of R¹³ is selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

In another aspect, the present disclosure provides a pharmaceutical composition comprising at least one therapeutic agent at least partially encapsulated in the polymer of the present disclosure. In certain embodiments, polymer comprises at least one selected from the group consisting of polyDEAA and polyNIPAM.

In another aspect, the present disclosure provides an adhesive composition comprising the polymer of the present disclosure. In certain embodiments, the adhesive composition comprises polyHEAA.

In another aspect, the present disclosure provides an ion-exchange membrane composition comprises the polymer of the present disclosure, wherein the polymer comprises at least one ionic substituent. In certain embodiments, the polymer comprises polyAPTMAT.

In another aspect, the present disclosure provides a battery comprising the ion-exchange membrane composition of the present disclosure.

In another aspect, the present disclosure provides a method of promoting stereocontrolled living radical polymerization reaction. In certain embodiments, the method comprises (a) contacting the compound of the disclosure and a Lewis acid to provide a Lewis acid-catalyst complex. In certain embodiments, the method comprises (b) contacting the Lewis acid-catalyst complex with at least two vinyl monomers to provide a mixture. In certain embodiments, each vinyl monomer is substituted with at least one substituent comprising a heteroatom having at least one coordinating lone pair of electrons. In certain embodiments, each vinyl monomer is identical. In certain embodiments, the method comprises (c) irradiating the mixture to provide a catalyst-nascent polymer complex.

### BRIEF DESCRIPTION OF THE FIGURES

The drawings illustrate generally, by way of example, but not by way of limitation, various embodiments of the present application.

FIGS. 1A-ID depict stereocontrolled radical polymerizations. FIG. 1A depicts inherent challenges of stereocontrol in radical polymerization. FIG. 1B depicts radical polymerization involving a chiral capping agent. FIG. 1C depicts Lewis acid (LA) assisted isotactic (living) radical polymerizations. FIG. 1D depicts chain-end stereocontrol directed by reversible radical deactivation, as described herein.

FIG. 2 provides a graph depicting ln[M]₀/[M] vs time profile during a periodic light-on-off process of an exemplary polymerization, wherein M is monomer and [M] stands for concentration of M.

FIGS. 3A-3B depict the stereochemistry of radical addition products obtained through chain-end control (FIG. 3A) and catalyst control (FIG. 3B) mechanisms. Note: σ₁ and σ₂ are the probability values that vary with the type of catalyst.

FIG. 4 depicts ¹H-NMR spectra of CD₃OD solution of (a) DMAA only, [DMAA]=1 mol/L; (b) TMTZC only, [TMTZC]=2.5 mmol/L; (c) TMTZC and La(OTf)₃, [TMTZC]=[La(OTf)₃]=2.5 mmol/L; (d) La(OTf)₃, DMAA, and TMTZC, [TMTZC]=[La(OTf)₃]=2.5 mmol/L, [DMAA]=0.025 mol/L; (e) La(OTf)₃, DMAA, and TMTZC, [TMTZC]=[La(OTf)₃]=2.5 mmol/L, [DMAA]=1 mol/L.

FIG. 5 depicts a proposed mechanism of cobalt-mediated radical polymerization.

FIG. 6 depicts kinetic plots of DMAA polymerizations initiated by 1a. Conditions: [DMAA]₀:[1a]₀=400:1 in MeOH at room temperature with a light intensity of 3 mW/cm². La(OTf)₃ mole fraction was relative to monomer.

FIG. 7 depicts kinetic plots of DMAA polymerizations initiated by 1e. [DMAA]₀:[1a]₀=400:1 in MeOH at room temperature with a light intensity of 3 mW/cm². La(OTf)₃ mole fraction was relative to monomer.

FIGS. 8A-8D depicting monomer conversion vs polymerization time in polymerization of DMAA initiated by (FIG. 8A) 1a and (FIG. 8B) 1e with various La(OTf)₃ as well as representative GPC traces (2.5 mol % La(OTf)₃) shown in (FIG. 8C) and (FIG. 8D), respectively. Conditions: [DMAA]₀:[initiator]₀=400:1 in MeOH at room temperature with a light intensity of 3 mW/cm². La(OTf)₃ mole fraction was relative to monomer.

FIGS. 9A-9D provide exemplary data relating to the polymerization of DEAA (FIGS. 9A-9B) and NIPAM (FIGS. 9C-9D). FIG. 9A depicts a plot of monomer conversion vs polymerization time for DEAA. FIG. 9B depicts GPC traces of polymerized DEAA. FIG. 9C depicts a plot of monomer conversion vs polymerization time for NIPAM. FIG. 9D depicts GPC traces of polymerized NIPAM. Conditions: [monomer]₀:[1e]₀:[La(OTf)₃]=400:1:4 in MeOH at room temperature with a light intensity of 3 mW/cm².

FIG. 10 provides a graph depicting GPC traces of diblock copolymers polytBA-b-polyDMAA before (a) and after (b) chain extension of a co-polytBa macroinitiator.

FIG. 11 provides a graph depicting GPC traces of diblock copolymers polytBA-b-polyDEAA before (a) and after (b) chain extension of a co-polytBa macroinitiator.

FIG. 12 provides a graph depicting GPC traces of diblock copolymers polyDMAA-b-polytBA before (a) and after (b) chain extension of a co-polyDMAA macroinitiator.

FIGS. 13A-13F depict LACoP-mediated LRPs in methanol. FIG. 13A depicts a general scheme and exemplary tacticity data for LACoP-initiated photopolymerization of acrylamides; [DMAA]₀:[initiator]₀=400:1. FIG. 13B depicts chemical structures of initiators and ligands designed in the studies. FIG. 13C depicts the scope of monomers and corresponding percentage of meso diads obtained from polymerizations initiated by 1e (no parentheses) and TPO (in parentheses) with 5 mol % La(OTf)₃. FIGS. 13D-13F depict ¹H-NMR spectra (FIG. 13D), DSC curves (FIG. 13E), and WAXS profiles (FIG. 13F) of polyDMAA with different degrees of tacticity (i.e., 95% in (a), 83% in (b), and 51% m (c)).

FIG. 14 provides a graph depicting DSC traces of polyHEAA with various degrees of tacticity.

FIGS. 15A-15G depicts kinetic and mechanistic studies of LACoP-mediated LRP.

FIG. 15A depicts a semi-logarithmic pseudo-first-order kinetic plot for an exemplary polymerization of DMAA at various loadings of La(OTf)₃. [DMAA]₀: [1e]₀=400:1. FIG. 15B depicts evolution of molecular weight and dispersity as a function of DMAA conversion.

FIG. 15C depicts exemplary GPC traces before and after chain extension of a polyDMAA macroinitiator with tBA. FIG. 15D provides a graph depicting apparent propagation rate coefficients and meso diads in 1a- and 1e-initiated LRPs. FIG. 15E provides a schematic description of chain-end-selective LA coordination in 1e-initiated polymerization and ¹H-NMR study of LA/TACN coordination. FIG. 15F depicts a plot of percentage of meso diads versus DMAA conversion. FIG. 15G depicts a proposed mechanism of 1a/1e-initiated polymerizations at different monomer conversions.

FIG. 16 provides a graph depicting lap shear stress-strain curves of polyHEAA and commercial adhesives.

FIG. 17 provides a graph depicting tacticity of polyDMAA with various water volume fraction. Conditions: [DMAA]₀:[1e]₀:[La(OTf)₃]=400:1:10 in 4 mL of MeOH/H₂O at room temperature with a light intensity of 3 mW/cm².

FIGS. 18A-18F depict tacticity-dependent properties of exemplary polymers of the present disclosure. FIGS. 18A-18B depict a general mechanism of LCST behavior (FIG. 18A) and the dependence of polyDEAA and polyNIPAM LCST on the degree of isotacticity (FIG. 18B). FIGS. 18C-18D depict an experimental setup for adhesion performance evaluation of polyHEAA (FIG. 18C) and exemplary summarized data on the tacticity impact (FIG. 18D).

FIGS. 18E-18F depict WAXS profiles (FIG. 18E) and ionic conductivity (FIG. 18F) of exemplary polyAPTMAT polymers with varied tacticity.

FIG. 19 depicts LCST curves of polyDEAA measured by UV-Vis spectroscopy.

FIG. 20 depicts LCST curves of polyNIPAM measured by UV-Vis spectroscopy.

FIG. 21 depicts LCST curves of exemplary 79% m polyDEAA polymers with differing molecular weights.

FIG. 22 depicts LCST curves of polyDEAA (79% m, 55.3 kDa) measured at different concentrations.

FIG. 23 depicts exemplary TGA and DTG curves for polyHEAA.

FIG. 24 depicts exemplary TGA and DTG curves for polyDMAA.

FIG. 25 depicts exemplary TGA and DTG curves for polyDEAA.

FIG. 26 depicts exemplary TGA and DTG curves for polyNIPAM.

FIG. 27 provides a graph depicting GPC traces of stereo-block polyDEAA before (a) and after (b) adding a Lewis acid.

FIG. 28 provides a graph depicting conversion of DMAA with various water volume fractions. Conditions: [DMAA]₀:[1e]₀:[La(OTf)₃]=400:1:10 in 4 mL of MeOH/H₂O at room temperature with a light intensity of 3 mW/cm².

FIG. 29 provides a graph and table depicting DSC traces and data for polyDMAA with various degrees of tacticity.

FIG. 30 provides a graph and table depicting DSC traces and data for polyDEAA with various degrees of tacticity.

FIG. 31 provides a graph and table depicting DSC traces and data for polyNIPAM with various degrees of tacticity.

FIG. 32 provides a graph and table depicting Nyquist plots and associated data for 78% m polyAPTMA (Mₙ=44 kDa, Ð=1.45) at different temperatures.

FIG. 33 depicts a comparison of the reported methods to achieve stereocontrolled FRPs.

FIG. 34 depicts an exemplary ¹H-NMR spectrum of atactic polyDMAA (2a) in DMSO-d₆ at room temperature.

FIG. 35 depicts an exemplary ¹H-NMR spectrum of isotactic polyDMAA (2a) in DMSO-d₆ at room temperature.

FIG. 36 depicts an exemplary ¹H-NMR spectrum of isotactic polyDMAA (2a) in DMSO-d₆ at room temperature.

FIG. 37 depicts an exemplary ¹H-NMR spectrum of isotactic polyDMAA (2a) in DMSO-d₆ at room temperature.

FIG. 38 depicts an exemplary ¹H-NMR spectrum of isotactic polyDMAA (2a) in DMSO-d₆ at room temperature.

FIG. 39 depicts an exemplary ¹H-NMR spectrum of atactic polyDEAA (2b) in DMSO-d₆ at 130° C.

FIG. 40 depicts an exemplary ¹H-NMR spectrum of isotactic polyDEAA (2b) in DMSO-d₆ at 130° C.

FIG. 41 depicts an exemplary ¹H-NMR spectrum of atactic polyNIPAM (2c) in DMSO-d₆ at 130° C.

FIG. 42 depicts an exemplary ¹H-NMR spectrum of isotactic polyNIPAM (2c) in DMSO-d₆ at 130° C.

FIG. 43 depicts an exemplary ¹H-NMR spectrum of atactic poly(4-Acryloylmorpholine) (2d) in DMSO-d₆ at 130° C.

FIG. 44 depicts an exemplary ¹H-NMR spectrum of isotactic poly(4-Acryloylmorpholine) (2d) in DMSO-d₆ at 130° C.

FIG. 45 depicts an exemplary ¹H-NMR spectrum of atactic poly(N-(3-methoxypropyl)acrylamide) (2e) in DMSO-d₆ at 130° C.

FIG. 46 depicts an exemplary ¹H-NMR spectrum of isotactic poly(N-(3-methoxypropyl)acrylamide) (2e) in DMSO-d₆ at 130° C.

FIG. 47 depicts an exemplary ¹H-NMR spectrum of atactic poly N-(2-(2-methoxyethoxy)ethyl)acrylamide (2f) in DMSO-d₆ at 130° C.

FIG. 48 depicts an exemplary ¹H-NMR spectrum of isotactic poly N-(2-(2-methoxyethoxy)ethyl)acrylamide (2f) in DMSO-d₆ at 130° C.

FIG. 49 depicts an exemplary ¹H-NMR spectrum of atactic poly N-(2-(2-(2-methoxyethoxy)ethoxy)ethyl)acrylamide (2g) in DMSO-d₆ at 130° C.

FIG. 50 depicts an exemplary ¹H-NMR spectrum of isotactic poly N-(2-(2-(2-methoxyethoxy)ethoxy)ethyl)acrylamide (2g) in DMSO-d₆ at 130° C.

FIG. 51 depicts an exemplary ¹H-NMR spectrum of atactic poly (N-(3-(dimethylamino)propyl)acrylamide) (2g) in DMSO-d₆ at 130° C.

FIG. 52 depicts an exemplary ¹H-NMR spectrum of isotactic poly (N-(3-(dimethylamino)propyl)acrylamide) (2g) in DMSO-d₆ at 130° C.

FIG. 53 depicts an exemplary H-NMR spectrum of atactic polyHEAA (2i) in DMSO-d₆ at 130° C.

FIG. 54 depicts an exemplary ¹H-NMR spectrum of isotactic polyHEAA (2i) in DMSO-d₆ at 130° C.

FIG. 55 depicts an exemplary ¹H-NMR spectrum of atactic poly((3-acrylamidopropyl)trimethylammonium trifluoromethanesulfonate) (2j) in D₂O at 95° C.

FIG. 56 depicts an exemplary ¹H-NMR spectrum of isotactic poly((3-acrylamidopropyl)trimethylammonium trifluoromethanesulfonate) (2j) in D₂O at 95° C.

FIG. 57 depicts an exemplary ¹H-NMR spectrum of isotactic poly 3-((3-acrylamidopropyl)dimethylammonio)propane-1-sulfonate (2k) in D₂O at 95° C.

### DETAILED DESCRIPTION OF THE DISCLOSURE

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y. or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B. or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

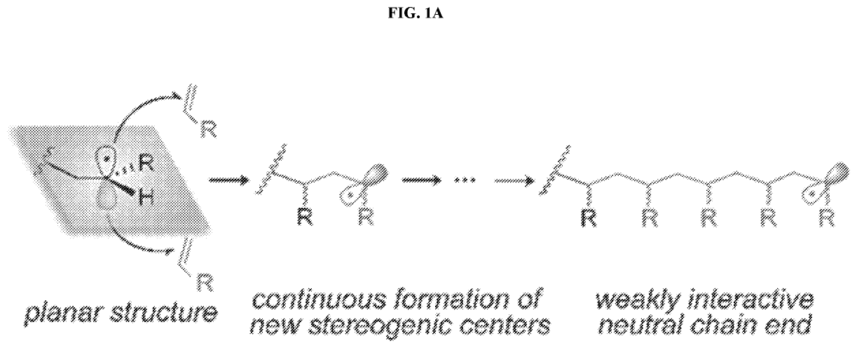

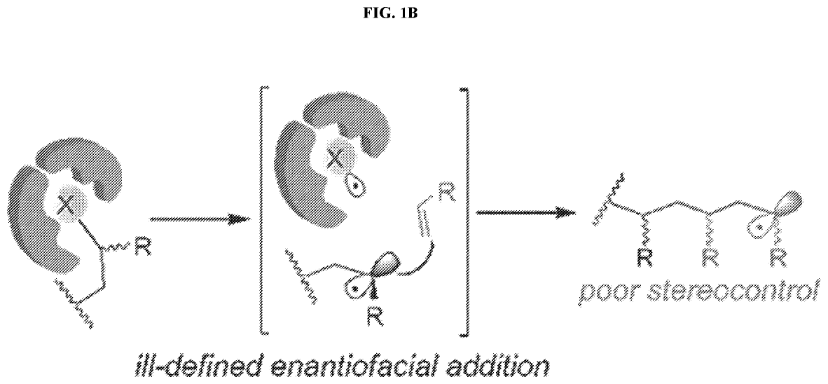

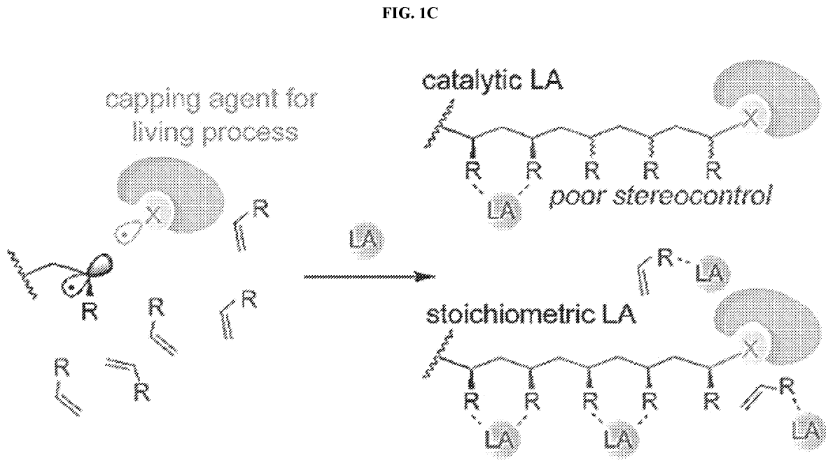

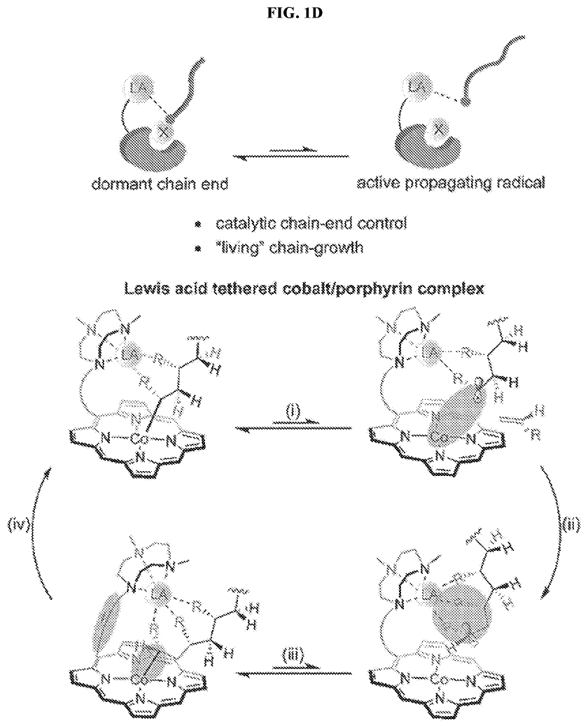

Development of stereocontrolled radical polymerization (RP) remains a fundamental challenge (FIG. 1A) primarily due to the difficulty in controlling the stereochemistry of the radical propagating chain ends with an sp²-hybridized planar geometry. Various attempts have been made to realize stereocontrol utilizing RP techniques. Recent development of living radical polymerization (LRP) techniques opened ways to synthesize polymers with controlled molecular weight, dispersity, and topology. Capping agents used in LRP to enable reversible radical deactivation and controlled chain propagation were designed with defined chirality for enantioselective monomer additions (FIG. 1B). However, the efficient chain end interactions that are required to ensure enantiofacially differentiated vinyl additions (e.g., organometallic bonding in coordination polymerization and electrostatic force in ionic polymerization) could not be retained in LRP between a non-bonding neutral radical chain end and a designed chiral capping agent, therefore resulting in negligible stereocontrol.

Stereoregular polymers have been synthesized through RP of chiral auxiliary monomers or in a rationally designed reaction environment (e.g., in the presence of fluoroalcohols or under spatial confinement). The addition of Lewis acids (LAs) has been widely demonstrated as a more general strategy to promote the stereocontrol in (L)RPs of common vinyl monomers under readily accessible reaction conditions. Polar vinyl monomers including methacrylates, acrylamides, and acrylonitrile were examined in this method and isotactic polymers with over 80% meso diads (% m) were obtained.

A prevalent rationale attributes the enhanced isotacticity to the hypothesis that LA binds to the polar pendant groups (i.e., esters, amides, and nitriles, of the terminal and penultimate enchained monomers) thereby forming a meso-configuration at the adjacently chelated units upon the subsequent monomer addition (FIG. 1C). However, large quantity of LAs, typically 10-20 mol % relative to the monomer, are necessary to ensure LA chelation selectively with the pendant groups at the growing chain ends due to the presence of numerous competing chelating species including non-chain-end pendant groups of polymers and unreacted monomers. Further application of this method is restricted by the high metallic LA loading and poor LA compatibility with reactive or chelating pendants of monomers.

Described herein, in part, is a molecular design of rare earth cobalt based bimetallic catalytic system to address the long-standing challenge in stereocontrolled RP (FIG. 1D) through a chain-end control mechanism. The cobalt center (i.e., organometallic cobalt(III)/porphyrin (R—Co^{III}/por) complex) is responsible for the initiation of a living radical chain-growth process. The C—Co^{III} bond undergoes photocatalytic homolytic cleavage to form a radical (i.e., R·) and a Co^{III}/por species (FIG. 1D). A multidentate cyclic ligand of aza-crown ether (ACE) that is covalently anchored with the stable R—Co^{III}/por binds to LA (e.g., rare earth metal salts) yielding an LA-tethered Co/por (LACoP) bimetallic complex.

Without wishing to be bound by theory, it has been hypothesized herein that the tethered LA is confined in proximity to the growing chain end, thereby providing a chain-end-selective LA chelation, followed by meso radical addition to an incoming monomer via a chain-end control mechanism (FIG. 1D). The in situ generated Co^{III}/por can reversibly deactivate the propagating radical to form an R—Co^{III}/por dormant species either before or after the monomer addition (FIG. 1D).

The hypothesized proximity-induced chain-end control can be facilitated by its covalently linked Co/por that mediates the rapid reversible chain-end capping with a deactivation rate constant above 10⁵ M⁻¹s⁻¹. Conversely, the chain-end-chelated LA can direct a Co/por to regulate the growth of the same polymer chain and suppress the interchain transfer of LACoP, which represents a competing process detrimental for a chain-end controlled mechanism.

Although each monomer addition event produces a new terminal unit, the subsequent recapping of radical by Co^{II}/por forces the tethered LA to rearrange to interact with the newest enchained monomers and ensure continuous meso monomer additions even at catalytic loading of LA (FIG. 1D). Whereas the concept of bimetallic catalysis involving covalently bridged hetero-catalytic centers has been applied in non-radical systems to attain high-performance polymerizations, leveraging this proximity effect to realize chain-end-controlled radical propagation remains unprecedented.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C≡CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines: R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “anionic ligand” or “X-type ligand” as used herein refers to a class of ligands that donate a single electron to a metal center and accept one electron from the metal when using the neutral ligand method of electron counting, or donate two electrons to the metal when using the donor pair method of electron counting. The “anionic” or “X-type” ligands yield a covalent bond with the metal center. Non-limiting examples of “anionic” and/or “X-type” ligands include H, halogens, OH, CN, alkyl, NO (bent), and C(═O)O(alkyl) ligands.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “arylenyl” as used herein refers to a bivalent aryl radical (e.g., 1,4-phenylene). In certain embodiments, the term may be regarded as a divalent radical formed by the removal of two hydrogen atoms from one or more rings of a aryl moiety, wherein the hydrogen atoms may be removed from the same or different rings, preferably the same ring.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl. 5-thiazolyl), pyridyl (2-pyridyl, 3-pyridyl. 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl). 5-(2,3-dihydro-benzo[b]thiophenyl), 6-(2,3-dihydro-benzo[b]thiophenyl). 7-(2,3-dihydro-benzo[b]thiophenyl), indolyl (1-indolyl. 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl, C₂-C₃ heterocycloalkyl, C₂-C₄ heterocycloalkyl, C₂-C₅ heterocycloalkyl, C₂-C₆ heterocycloalkyl, C₂-C₇ heterocycloalkyl, C₂-C₈ heterocycloalkyl, C₂-C₉ heterocycloalkyl. C₂-C₁₀ heterocycloalkyl. C₂—CH heterocycloalkyl, and the like, up to and including a C₂-14₅ heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “ion exchange membrane” as used herein refers to a membrane comprising chemical groups capable of combining with ions or exchanging ions between the membrane and an external medium. The chemical groups can be in a form of a salt, an acid or a base, wherein the cations, anions, protons or hydroxyl ions thereof are exchangeable with other cations, anions, protons or hydroxyl ions from an external source (e.g., a solution or gas). Ion exchange membranes can be provided in an acid form and converted to a salt form by pretreating the membrane with a base, such as an alkali metal salt or an alkaline earth metal salt or in an alkaline form, being thereafter converted to a salt by pretreating the membrane with a suitable acid.

The term “Lewis acid” as used herein refers to any species having an empty orbital that can accept a pair of electrons and form a coordinate covalent bond.

The term “lone pair” or “lone pair of electrons” as used herein refers to a pair of electrons in the outermost shell of an atom (e.g., O, N, S, and P) that are not used in bonding.

The term “nascent,” as used herein in the context of a polymer, refers to a reactive intermediate of a polymer which is either undergoing, or may undergo, further chain extension by virtue of a covalent bond between the polymer backbone and a transition metal (i.e., a portion of a polymer that is in the process of being synthesized).

The term “rare earth metal” as used herein refers to seventeen chemical elements in the periodic table, which includes fifteen lanthanides (i.e., fifteen elements having atomic numbers in the range from 57 to 71, from lanthanum to lutetium) in addition to scandium and yttrium.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters: a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “tacticity” as used herein generally refers to the stereoregularity of a polymer. For example, the chirality of adjacent monomers can be of either like or opposite configuration. The term “diad” is used to designate two contiguous monomers, whereas the term “triad” is used to designate three adjacent monomers. If the chirality of adjacent monomers is of the same relative configuration, the diad is defined as meso (m); if opposite in configuration, it is termed racemo (r) diad. When three adjacent monomers are of the same configuration, the stereoregularity of the triad is ‘mm’. If two adjacent monomers in a three-monomer sequence have the same chirality and that is different from the relative configuration of the third unit, this triad has ‘mr’ tacticity. An ‘rr’triad has the middle monomer unit having an opposite configuration from either neighbor. The fraction of each type of triad in the polymer can be determined and when multiplied by 100 indicates the percentage of that type found in the polymer. Another way to describe the configurational relationship is to term polymers with contiguous monomer pairs having the same chirality as isotactic polymers and those of contiguous monomer pairs having opposite chirality syndiotactic polymers. If the chirality of adjacent monomers is random, the polymer is referred to as an atactic polymer.

### Compositions

### Catalysts

In one aspect, the present disclosure provides a compound of Formula (Ia), or a salt, solvate, stereoisomer, or isotopologue thereof:

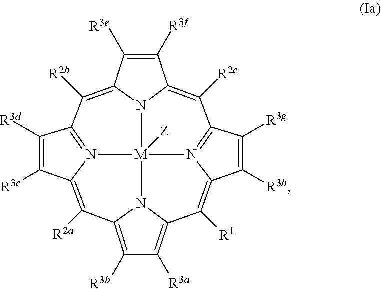

wherein:
M is Co;
Z is absent or an anionic ligand;
R¹ is

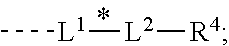

R^{2a}, R^{2b}, and R^{2c} are each independently selected from the group consisting of H, optionally substituted C₁-C₆ alkyl, optionally substituted C₃-C₈ cycloalkyl, optionally substituted C₆-C₁₀ aryl and optionally substituted C₂-C₁₀ heteroaryl;
R^{3a}, R^{3b}, R^{3c}, R^{3d}, R^{3e}, R^{3f}, R^{3g}, and R^{3h} are each independently selected from the group consisting of H and optionally substituted C₁-C₆ alkyl;
L¹ is selected from the group consisting of -(optionally substituted C₆-C₁₀ arylenyl)-* and -(optionally substituted C₂-C₁₀ heteroarylenyl)-*,
   wherein a substituent of the C₆-C₁₀ arylenyl or the C₂-C₁₀ heteroarylenyl in L¹ can combine with a substituent of the C₆-C₁₀ aryl or C₂-C₁₀ heteroaryl in
   R^{2b} to form an optionally substituted C₂₀-C₃₀ heterocycloalkenyl;
L² is selected from the group consisting of *—C(═O)N(R⁵)(optionally substituted C₁-C₆ alkylenyl)-, *—C(═O)O(optionally substituted C₁-C₆ alkylenyl)-, *—C(═O)(optionally substituted C₁-C₆ alkylenyl)-, *-(optionally substituted C₁-C₆ alkylenyl)-, *—N(R⁵)C(═O)(optionally substituted C₁-C₆ alkylenyl)-, *—OC(═O)(optionally substituted C₁-C₆ alkylenyl)-, *—C(═O)N(R⁵)(optionally substituted C₁-C₆ heteroalkylenyl)-, *—C(═O)O(optionally substituted C₁-C₆ heteroalkylenyl)-, *—C(═O)(optionally substituted C₁-C₆ heteroalkylenyl)-, *-(optionally substituted C₁-C₆ heteroalkylenyl)-, *—N(R⁵)C(═O)(optionally substituted C₁-C₆ heteroalkylenyl)-, and *—OC(═O)(optionally substituted C₁-C₆ heteroalkylenyl)-;
R⁴ is an optionally substituted C₆-C₈ heterocycloalkyl,
   wherein the C₆-C₈ heterocycloalkyl comprises at least three atoms independently selected from the group consisting of N, O, and S; and
each occurrence of R⁵ is independently selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

In certain embodiments, the compound of Formula (Ia) is a compound of Formula (Ia-1):

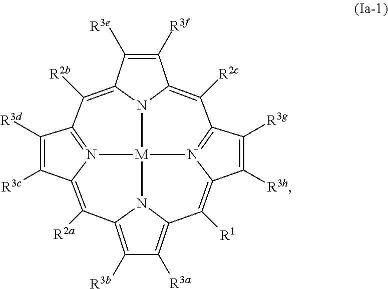

wherein:
M is Co;
R¹ is

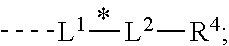

R^{2a}, R^{2b}, and R^{2e} are each independently selected from the group consisting of H, optionally substituted C₁-C₆ alkyl, optionally substituted C₃-C₈ cycloalkyl, optionally substituted C₆-C₁₀ aryl and optionally substituted C₂-C₁₀ heteroaryl;
R^{3a}, R^{3b}, R^{3c}, R^{3d}, R^{3e}, R^{3f}, R^{3g}, and R^{3h} are each independently selected from the group consisting of H and optionally substituted C₁-C₆ alkyl;
L¹ is selected from the group consisting of -(optionally substituted C₆-C₁₀ arylenyl)-* and -(optionally substituted C₂-C₁₀ heteroarylenyl)-*,
   wherein a substituent of the C₆-C₁₀ arylenyl or the C₂-C₁₀ heteroarylenyl in Lt can combine with a substituent of the C₆-C₁₀ aryl or C₂-C₁₀ heteroaryl in R^{2b} to form an optionally substituted C₂₀-C₃₀ heterocycloalkenyl;
L² is selected from the group consisting of *—C(═O)N(R⁵)(optionally substituted C₁-C₆ alkylenyl)-, *—C(═O)O(optionally substituted C₁-C₆ alkylenyl)-, *—C(═O)(optionally substituted C₁-C₆ alkylenyl)-, *-(optionally substituted C₁-C₆ alkylenyl)-, *—N(R⁵)C(═O)(optionally substituted C₁-C₆ alkylenyl)-, *—OC(═O)(optionally substituted C₁-C₆ alkylenyl)-, *—C(═O)N(R⁵)(optionally substituted C₁-C₆ heteroalkylenyl)-, *—C(═O)O(optionally substituted C₁-C₆ heteroalkylenyl)-, *—C(═O)(optionally substituted C₁-C₆ heteroalkylenyl)-, *-(optionally substituted C₁-C₆ heteroalkylenyl)-, *—N(R⁵)C(═O)(optionally substituted C₁-C₆ heteroalkylenyl)-, and *—OC(═O)(optionally substituted C₁-C₆ heteroalkylenyl)-;
R⁴ is an optionally substituted C₆-C₈ heterocycloalkyl,
   wherein the C₆-C₈ heterocycloalkyl comprises at least three atoms independently selected from the group consisting of N, O, and S; and
each occurrence of R⁵ is independently selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

In certain embodiments, the compound of Formula (Ia) is a compound of Formula (Ia-2):

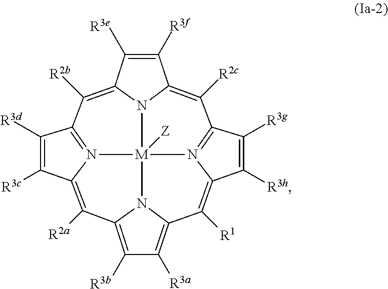

wherein:
M is Co;
Z is an anionic ligand;
R¹ is

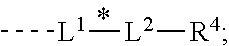

R^{2a}, R^{2b}, and R^{2c} are each independently selected from the group consisting of H, optionally substituted C₁-C₆ alkyl, optionally substituted C₃-C₈ cycloalkyl, optionally substituted C₆-C₁₀ aryl and optionally substituted C₂-C₁₀ heteroaryl;
R^{3a}, R^{3b}, R^{3c}, R^{3d}, R^{3e}, R^{3f}, R^{3g}, and R^{3h} are each independently selected from the group consisting of H and optionally substituted C₁-C₆ alkyl;
L¹ is selected from the group consisting of -(optionally substituted C₆-C₁₀ arylenyl)-* and -(optionally substituted C₂-C₁₀ heteroarylenyl)-*,
   wherein a substituent of the C₆-C₁₀ arylenyl or the C₂-C₁₀ heteroarylenyl in L¹ can combine with a substituent of the C₆-C₁₀ aryl or C₂-C₁₀ heteroaryl in R²¹ to form an optionally substituted C₂₀-C₃₀ heterocycloalkenyl;
L² is selected from the group consisting of *—C(═O)N(R⁵)(optionally substituted C₁-C₆ alkylenyl)-, *—C(═O)O(optionally substituted C₁-C₆ alkylenyl)-, *—C(═O)(optionally substituted C₁-C₆ alkylenyl)-, *-(optionally substituted C₁-C₆ alkylenyl)-, *—N(R⁵)C(═O)(optionally substituted C₁-C₆ alkylenyl)-, *—OC(═O)(optionally substituted C₁-C₆ alkylenyl)-, *—C(═O)N(R⁵)(optionally substituted C₁-C₆ heteroalkylenyl)-, *—C(═O)O(optionally substituted C₁-C₆ heteroalkylenyl)-, *—C(═O)(optionally substituted C₁-C₆ heteroalkylenyl)-, *-(optionally substituted C₁-C₆ heteroalkylenyl)-, *—N(R⁵)C(═O)(optionally substituted C₁-C₆ heteroalkylenyl)-, and *—OC(═O)(optionally substituted C₁-C₆ heteroalkylenyl)-;
R⁴ is an optionally substituted C₆-C₈ heterocycloalkyl,
   wherein the C₆-C₈ heterocycloalkyl comprises at least three atoms independently selected from the group consisting of N, O, and S; and
each occurrence of R⁵ is selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

In certain embodiments, the compound is a compound of Formula (Ib), or a salt, solvate, stereoisomer, or isotopologue thereof:

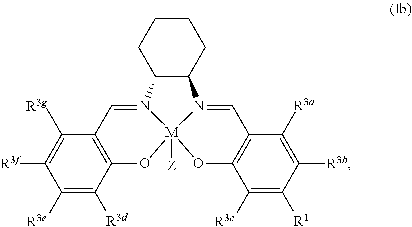

M is Co;
Z is absent or an anionic ligand;
R¹ is

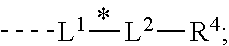

R^{3a}, R^{3b}, R^{3c}, R^{3d}, R^{3e}, R^{3f}, and R^{3g} are each independently selected from the group consisting of H and optionally substituted C₁-C₆ alkyl;
L¹, if present, is selected from the group consisting of -(optionally substituted C₆-C₁₀ arylenyl)-* and -(optionally substituted C₂-C₁₀ heteroarylenyl)-*;
L², if present, is selected from the group consisting of *—C(═O)N(R⁵)(optionally substituted C₁-C₆ alkylenyl)-, *—C(═O)O(optionally substituted C₁-C₆ alkylenyl)-, *—C(═O)(optionally substituted C₁-C₆ alkylenyl)-, *-(optionally substituted C₁-C₆ alkylenyl)-, *—N(R⁵)C(═O)(optionally substituted C₁-C₆ alkylenyl)-, *—OC(═O)(optionally substituted C₁-C₆ alkylenyl)-, *—C(═O)N(R⁵)(optionally substituted C₁-C₆ heteroalkylenyl)-, *—C(═O)O(optionally substituted C₁-C₆ heteroalkylenyl)-, *—C(═O)(optionally substituted C₁-C₆ heteroalkylenyl)-, *-(optionally substituted C₁-C₆ heteroalkylenyl)-, *—N(R⁵)C(═O)(optionally substituted C₁-C₆ heteroalkylenyl)-, and *—OC(═O)(optionally substituted C₁-C₆ heteroalkylenyl)-;
R⁴ is an optionally substituted C₆-C₈ heterocycloalkyl,
   wherein the C₆-C₈ heterocycloalkyl comprises at least three atoms selected from the group consisting of N, O, and S; and
each occurrence of R⁵, if present, is independently selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

In certain embodiments, the compound is a compound of Formula (Ic), or a salt, solvate, stereoisomer, or isotopologue thereof:

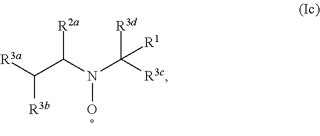

wherein:
R¹ is

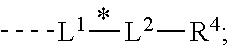

R^{2a} is selected from the group consisting of H, optionally substituted C₁-C₆ alkyl, optionally substituted C₃-C₈ cycloalkyl, optionally substituted C₆-C₁₀ aryl and optionally substituted C₂-C₁₀ heteroaryl;
R^{3a}, R^{3b}, R^{3c}, and R^{3d} are each independently selected from the group consisting of H and optionally substituted C₁-C₆ alkyl;
L¹, if present, is selected from the group consisting of -(optionally substituted C₆-C₁₀ arylenyl)-* and -(optionally substituted C₂-C₁₀ heteroarylenyl)-*;
L², if present, is selected from the group consisting of *—C(═O)N(R⁵)(optionally substituted C₁-C₆ alkylenyl)-, *—C(═O)O(optionally substituted C₁-C₆ alkylenyl)-, *—C(═O)(optionally substituted C₁-C₆ alkylenyl)-, *-(optionally substituted C₁-C₆ alkylenyl)-, *—N(R⁵)C(═O)(optionally substituted C₁-C₆ alkylenyl)-, *—OC(═O)(optionally substituted C₁-C₆ alkylenyl)-, *—C(═O)N(R⁵)(optionally substituted C₁-C₆ heteroalkylenyl)-, *—C(═O)O(optionally substituted C₁-C₆ heteroalkylenyl)-, *—C(═O)(optionally substituted C₁-C₆ heteroalkylenyl)-, *-(optionally substituted C₁-C₆ heteroalkylenyl)-, *—N(R⁵)C(═O)(optionally substituted C₁-C₆ heteroalkylenyl)-, and *—OC(═O)(optionally substituted C₁-C₆ heteroalkylenyl)-;
R⁴ is an optionally substituted C₆-C₈ heterocycloalkyl,
   wherein the C₆-C₈ heterocycloalkyl comprises at least three atoms selected from the group consisting of N, O, and S; and
each occurrence of R⁵, if present, is independently selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

In certain embodiments, the compound is a compound of Formula (Id), or a salt,

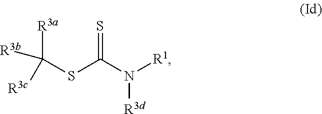

wherein:
R¹ is

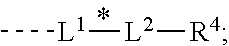

R^{3a}, R^{3b}, R^{3c}, and R^{3d} are each independently selected from the group consisting of H and optionally substituted C₁-C₆ alkyl;
L¹, if present, is selected from the group consisting of -(optionally substituted C₆-C₁₀ arylenyl)-* and -(optionally substituted C₂-C₁₀ heteroarylenyl)-*;
L², if present, is selected from the group consisting of *—C(═O)N(R⁵)(optionally substituted C₁-C₆ alkylenyl)-, *—C(═O)O(optionally substituted C₁-C₆ alkylenyl)-, *—C(═O)(optionally substituted C₁-C₆ alkylenyl)-, *-(optionally substituted C₁-C₆ alkylenyl)-, *—N(R⁵)C(═O)(optionally substituted C₁-C₆ alkylenyl)-, *—OC(═O)(optionally substituted C₁-C₆ alkylenyl)-, *—C(═O)N(R⁵)(optionally substituted C₁-C₆ heteroalkylenyl)-, *—C(═O)O(optionally substituted C₁-C₆ heteroalkylenyl)-, *—C(═O)(optionally substituted C₁-C₆ heteroalkylenyl)-, *-(optionally substituted C₁-C₆ heteroalkylenyl)-, *—N(R⁵)C(═O)(optionally substituted C₁-C₆ heteroalkylenyl)-, and *—OC(═O)(optionally substituted C₁-C₆ heteroalkylenyl)-;
R⁴ is an optionally substituted C₆-C₈ heterocycloalkyl,
   wherein the C₆-C₅ heterocycloalkyl comprises at least three atoms selected from the group consisting of N, O, and S; and
      each occurrence of R⁵, if present, is independently selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

In certain embodiments, the compound is a compound of Formula (Ie), or a salt, solvate, stereoisomer, or isotopologue thereof:

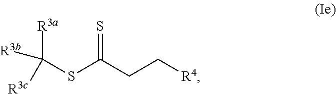

wherein:
R^{3a}, R^{3b}, and R^{3c} are each independently selected from the group consisting of H and optionally substituted C₁-C₆ alkyl; and
R⁴ is an optionally substituted C₆-C₈ heterocycloalkyl,
   wherein the C₆-C₈ heterocycloalkyl comprises at least three atoms selected from the group consisting of N, O, and S.

In certain embodiments, the compound is a compound of Formula (If), or a salt, solvate, stereoisomer, or isotopologue thereof:

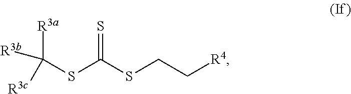

wherein:
R^{3a}, R^{3b}, and R^{3c} are each independently selected from the group consisting of H and optionally substituted C₁-C₆ alkyl; and
R⁴ is an optionally substituted C₆-C₈ heterocycloalkyl,
   wherein the C₆-C₈ heterocycloalkyl comprises at least three atoms selected from the group consisting of N, O, and S.

In certain embodiments, the compound is a compound of Formula (Ig), or a salt, solvate, stereoisomer, or isotopologue thereof:

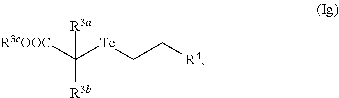

wherein:
R^{3a}, R^{3b}, and R^{3c} are each independently selected from the group consisting of H and optionally substituted C₁-C₆ alkyl; and
R⁴ is an optionally substituted C₆-C₈ heterocycloalkyl,
   wherein the C₆-C₅ heterocycloalkyl comprises at least three atoms selected from the group consisting of N, O, and S.

In certain embodiments, L¹ is selected from the group consisting of:

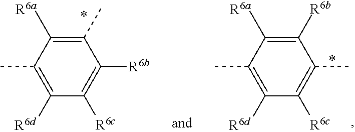

wherein R^{6a}, R^{6b}, R^{6c}, and R^{6d} are each independently selected from the group consisting of H, optionally substituted C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵.

In certain embodiments, at least one of R^{6a}, R^{6b}, R^{6c}, and R^{6d} is H. In certain embodiments, at least two of R^{6a}, R^{6b}, R^{6c}, and R^{6d} are H. In certain embodiments, at least three of R^{6a}, R^{6b}, R^{6c}, and R^{6d} are H. In certain embodiments, each of R^{6a}, R^{6b}, R^{6c}, and R^{6d} are H.

In certain embodiments, L² is *—C(═O)NH(C₁-C₆ alkylenyl)-.

In certain embodiments, L² is *—C(═O)NHCH₂CH₂—.

In certain embodiments, R⁴ is:

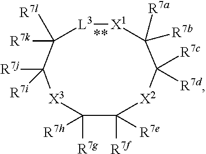

wherein:
L³ is selected from the group consisting of a bond and —X⁴—C(R^{7m})(R⁷ⁿ)—C(R^{7o})(R^{7p})—**;
X¹, X², X³, and X⁴, if present, are each independently selected from the group consisting of N(R⁵) and O;
R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, if present, are each independently selected from the group consisting of H, C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵,
   wherein one of R^{7a}, R^{7b}, R^{7c}, R^{7d}, R^{7e}, R^{7f}, R^{7g}, R^{7h}, R⁷ⁱ, R^{7j}, R^{7k}, and R^{7l} is -L²-*, or
   one of X¹. X², and X³ is N-L²-*; and
R⁸ is selected from the group consisting of H and optionally substituted C₁-C₆ alkyl.

In certain embodiments, X¹ is N-L²-*. In certain embodiments, X² is N-L²-*. In certain embodiments, X³ is N-L²-*.

In certain embodiments, at least one of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, is H. In certain embodiments, at least two of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least three of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least four of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least five of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least six of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least seven of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least eight of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least nine of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least ten of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least eleven of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least twelve of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least thirteen of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least fourteen of R^{7a}. R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least fifteen of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, each of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H.

In certain embodiments, R⁴ is

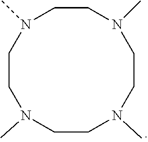

In certain embodiments, R⁴ is

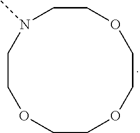

In certain embodiments, R⁴ is and

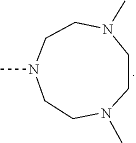

In certain embodiments, R^{2a}, R^{2b}, and R^{2d} are each independently:

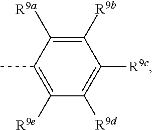

wherein R^{9a}, R^{9b}, R^{9c}, R^{9d}, and R^{9e} are each independently selected from the group consisting of H, optionally substituted C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵.

In certain embodiments, R^{9a} is H. In certain embodiments, R^{9a} is CH₃. In certain embodiments, R^{9b} is H. In certain embodiments, R^{9b} is CH₃. In certain embodiments. R^{9c} is H. In certain embodiments, R^{9c} is CH₃. In certain embodiments, R^{9d} is H. In certain embodiments, R^{9d} is CH₃. In certain embodiments, R^{9e} is H. In certain embodiments, R^{9e} is CH₃.

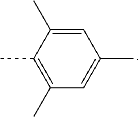

In certain embodiments, R^{2a} is

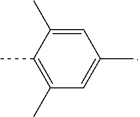

In certain embodiments, R^{2b} is

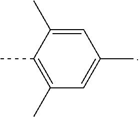

In certain embodiments, at least one of R^{3a}, R^{3b}, R^{3c}, R^{3d}, R^{3e}, R^{3f}, R^{3g}, and R³ is H. In certain embodiments, at least two of R^{3a}, R^{3b}, R^{3c}, R^{3d}, R^{3e}, R^{3f}, R^{3g}, and R^{3h} are H. In certain embodiments, at least three of R^{3a}, R^{3b}, R^{3c}, R^{3d}, R^{3e}, R^{3f}, R^{3g}, and R^{3h} are H. In certain embodiments, at least four of R^{3a}, R^{3b}, R^{3c}, R^{3d}, R^{3e}, R^{3f}, R^{3g}, and R^{3h} are H. In certain embodiments, at least five of R^{3a}, R^{3b}, R^{3c}, R^{3d}, R^{3e}, R^{3f}, R^{3g}, and R^{3h} are H. In certain embodiments, at least six of R^{3a}, R^{3b}, R^{3c}, R^{3d}, R^{3e}, R^{3f}, R^{3g}, and R^{3h} are H. In certain embodiments, at least seven of R^{3a}, R^{3b}, R^{3c}, R^{3d}, R^{3e}, R^{3f}, R^{3g}, and R^{3h} are H. In certain embodiments, each of R^{3a}, R^{3b}, R^{3c}, R^{3d}, R^{3e}, R^{3f}, R^{3g}, and R^{3h} are H.

In certain embodiments, Z is absent.

In certain embodiments, the compound is

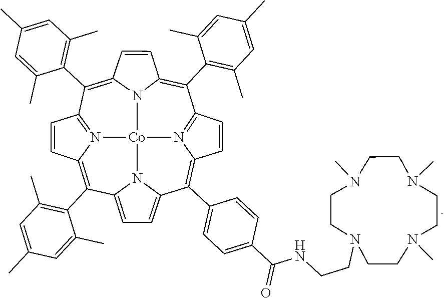

In certain embodiments, the compound is

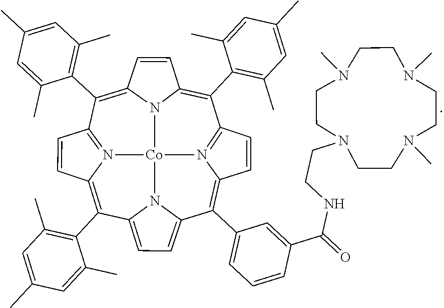

In certain embodiments, the compound is

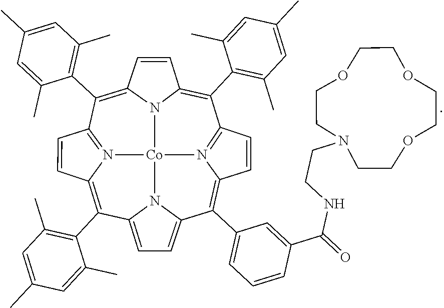

In certain embodiments, the compound is

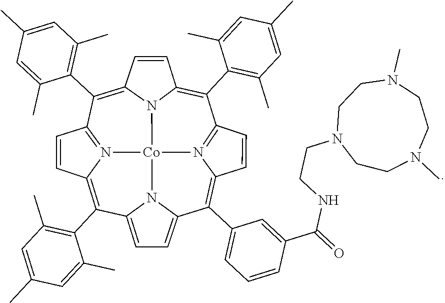

In certain embodiments, the compound is

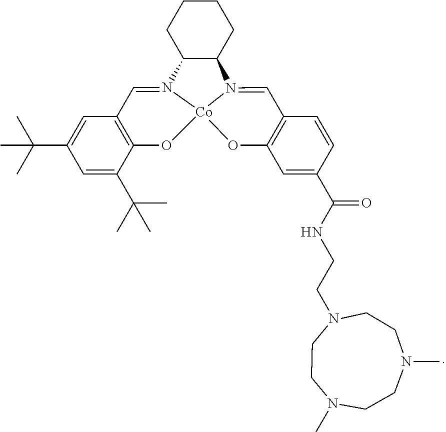

In certain embodiments, the compound is

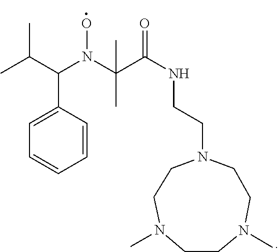 [SMILES: CC(C)C(c1ccccc1)N(O)C(C)(C)C(=O)NCCN1CCN(C)CCN(C)CC1]

In certain embodiments, the compound is

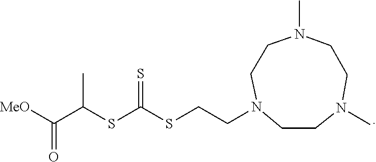 [SMILES: COC(=O)C(C)SC(=S)SCCN1CCN(C)CCN(C)CC1]

In certain embodiments, the compound is

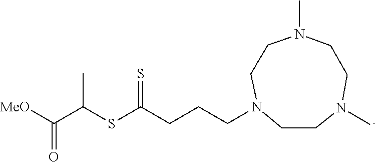 [SMILES: COC(=O)C(C)SC(=S)CCCN1CCN(C)CCN(C)CC1]

In certain embodiments, the compound is

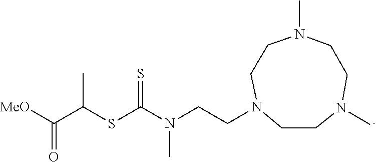 [SMILES: COC(=O)C(C)SC(=S)N(C)CCN1CCN(C)CCN(C)CC1]

In certain embodiments, the compound is

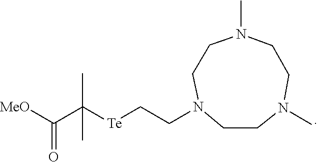

In certain embodiments, Z is C(═O)OR⁵. In certain embodiments, Z is optionally substituted alkyl. In certain embodiments, Z is halogen. In certain embodiments, Z is C(═O)OMe.

In certain embodiments, the compound is

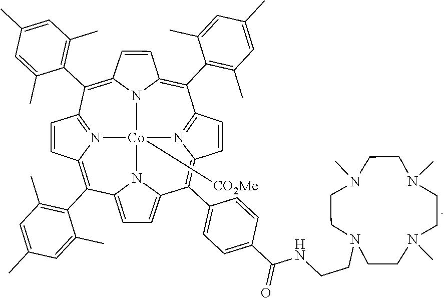

In certain embodiments, the compound is

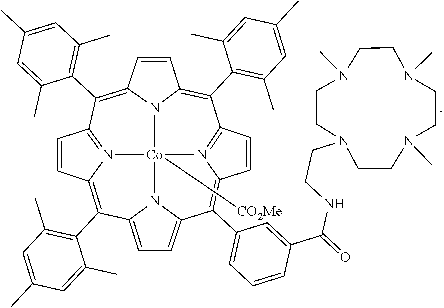

In certain embodiments, the compound is

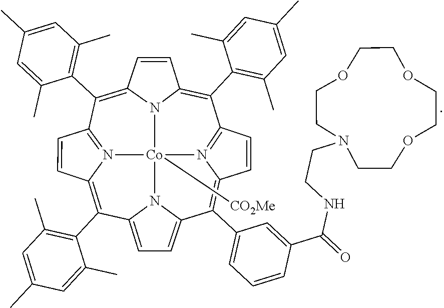

In certain embodiments, the compound is

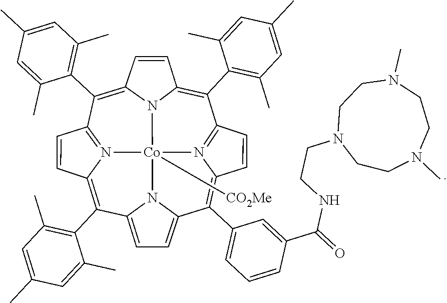

### Polymers

In another aspect, the disclosure provides a polymer composition of Formula (III):

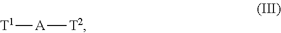

wherein:
each occurrence of T¹ is independently selected from the group consisting of C(═O)OR¹³, optionally substituted alkyl, and halogen,
T² is selected from the group consisting of H

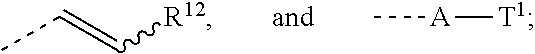

each occurrence of A independently comprises o units of

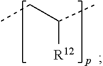

each occurrence o is independently an integer ranging from 1 to 10;
each occurrence of p is independently an integer ranging from 1 to 5,000;
R¹² is substituted with at least one substituent comprising a heteroatom having at least one coordinating lone pair of electrons,
   wherein the R¹² groups in each unit of

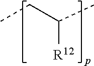

are identical; and
each occurrence of R¹³ is selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

In certain embodiments, the compound of Formula (III) is

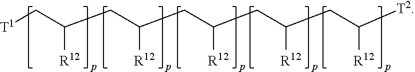

In certain embodiments, the compound of Formula (III) is

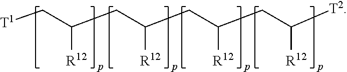

In certain embodiments, the compound of Formula (III) is

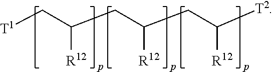

In certain embodiments, the compound of Formula (III) is

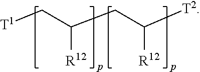

In certain embodiments, the compound of Formula (III) is

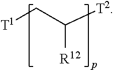

In certain embodiments, T¹ is C(═O)OMe.

In certain embodiments, R¹² in each unit of

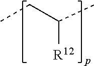

is selected from the group consisting of C(═O)N(R^{14a})(R^{14b}), C(═O)OR^{13a}, and CN;
R^{14a} and R^{14b}, if present, are independently selected from the group consisting of H, optionally substituted C₁-C₁₂ alkyl, optionally substituted C₁-C₁₂ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or
   wherein R^{14a} and R^{14b} can combine with the nitrogen atom to which they are bound to form an optionally substituted C₂-C₈ heterocycloalkyl or C₂-C₁₀ heteroaryl.

In certain embodiments, the R^{14a} in a unit of

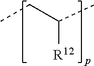

is C₁-C₆ alkyl. In certain embodiments, the R^{14a} in a unit of

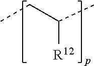

is C₁-C₆ hydroxyalkyl. In certain embodiments, the R^{14a} in a unit of

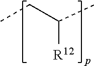

is C₁-C₁₂ alkoxyalkyl. In certain embodiments, the R^{14a} in a unit of

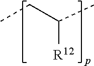

is C₁-C₆ aminoalkyl.

In certain embodiments, the R^{14b} in a unit of

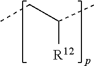

is C₁-C₆ alkyl. In certain embodiments, the R^{14b} in a unit of

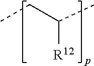

is C₁-C₆ hydroxyalkyl. In certain embodiments, the R^{14b} in a unit of

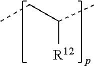

is C₁-C₁₂ alkoxyalkyl. In certain embodiments, the R^{14b} in a unit of

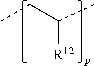

is C₁-C₆ aminoalkyl.

In certain embodiments, R^{14a} in a unit of

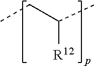

is methyl. In certain embodiments, R^{14a} in a unit of

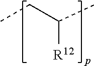

is ethyl. In certain embodiments. R^{14a} in a unit of

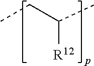

is isopropyl. In certain embodiments, R^{14a} in a unit of

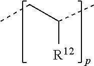

is —CH₂CH₂OH. In certain embodiments, R^{14a} in a unit of

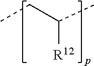

is —(CH₂CH₂O)₂₋₃CH₃. In certain embodiments, R^{14a} in a unit of

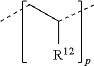

is —CH₂CH₂CH₂OCH₃. In certain embodiments, R^{14a} in a unit of

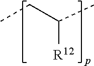

is —CH₂CH₂CH₂N(CH₃)₂. In certain embodiments, R^{14a} in a unit of

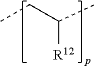

is —CH₂CH₂CH₂N(CH₃)₃⁺.

In certain embodiments, R^{14b} in a unit of

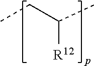

is methyl. In certain embodiments, R^{14b} in a unit of

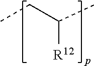

is ethyl. In certain embodiments, R^{14b} in a unit of

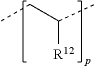

is isopropyl. In certain embodiments. R^{14b} in a unit of

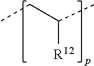

is —CH₂CH₂OH. In certain embodiments R^{4b} in a unit of

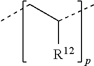

is —(CH₂CH₂O)₂₋₃CH₃. In certain embodiments, R^{14a} in a unit of

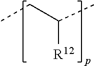

is —CH₂CH₂CH₂OCH₃. In certain embodiments, R^{14b} in a unit of

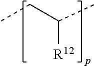

is —CH₂CH₂CH₂N(CH₃)₂. In certain embodiments, R^{14b} in a unit of

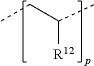

is —CH₂CH₂CH₂N(CH₃)₃⁺.

In certain embodiments, R¹² in a unit of

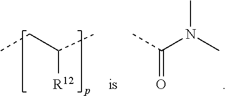

in certain embodiments, R¹² in a unit of

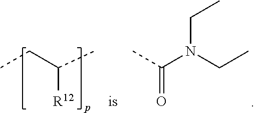

In certain embodiments. R¹² in a unit of

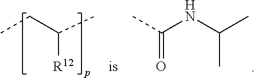

In certain embodiments, R¹² in a unit of

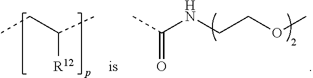

In certain embodiments, R in a unit of is

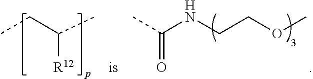

In certain embodiments, R¹² in a unit of

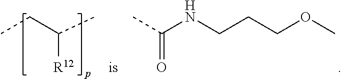

In certain embodiments, R¹² in a unit of

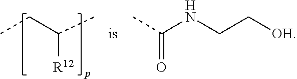

In certain embodiments, R¹² in a unit of

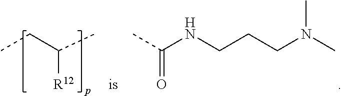

In certain embodiments, R¹² in a unit of

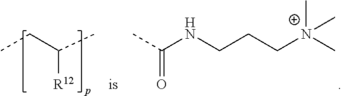

In certain embodiments, R¹² in a unit of

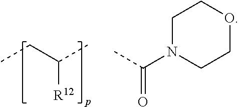

is

In certain embodiments, the polymer has a tacticity selected from the group consisting of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and about 99% m.

In certain embodiments, the polymer is prepared according to the methods of the present disclosure.

In another aspect, the present disclosure provides a pharmaceutical composition comprising at least one therapeutic agent at least partially encapsulated in the polymer of the present disclosure. In certain embodiments, polymer comprises at least one selected from the group consisting of polyDEAA and polyNIPAM.

In another aspect, the present disclosure provides an adhesive composition comprising the polymer of the present disclosure. In certain embodiments, the adhesive composition comprises polyHEAA.

In another aspect, the present disclosure provides an ion-exchange membrane composition comprises the polymer of the present disclosure, wherein the polymer comprises at least one ionic substituent. In certain embodiments, the polymer comprises polyAPTMAT.

In another aspect, the present disclosure provides a battery comprising the ion-exchange membrane composition of the present disclosure.

### Methods

In another aspect, the present disclosure provides a method of promoting stereocontrolled living radical polymerization reaction. In certain embodiments, the method comprises (a) contacting the compound of the disclosure and a Lewis acid to provide a Lewis acid-catalyst complex. In certain embodiments, the method comprises (b) contacting the Lewis acid-catalyst complex with at least two vinyl monomers to provide a mixture. In certain embodiments, each vinyl monomer is substituted with at least one substituent comprising a heteroatom having at least one coordinating lone pair of electrons. In certain embodiments, each vinyl monomer is identical. In certain embodiments, the method comprises (c) irradiating the mixture to provide a catalyst-nascent polymer complex.

In certain embodiments, the compound is a compound of Formula (Ia-2):

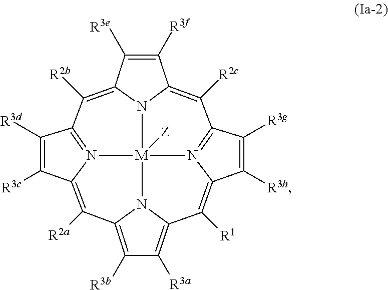

wherein:
M is Co;
Z is an anionic ligand;
R¹ is

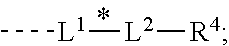

R^{2a}, R^{2b}, and R^{2c} are each independently selected from the group consisting of H, optionally substituted C₁-C₆ alkyl, optionally substituted C₃-C₈ cycloalkyl, optionally substituted C₆-C₁₀ aryl, and optionally substituted C₂-C₁₀ heteroaryl;
R^{3a}, R^{3b}, R^{3c}, R^{3d}, R^{3e}, R^{3f}, R^{3g}, and R^{3h} are each independently selected from the group consisting of H and optionally substituted C₁-C₆ alkyl;
L¹ is selected from the group consisting of -(optionally substituted C₆-C₁₀ arylenyl)-* and -(optionally substituted C₂-C₁₀ heteroarylenyl)-*,
   wherein a substituent of the C₆-C₁₀ arylenyl or the C₂-C₁₀ heteroarylenyl in L¹ can combine with a substituent of the C₆-C₁₀ aryl or C₂-C₁₀ heteroaryl in R^{2b} to form an optionally substituted C₂₀-C₃₀ heterocycloalkenyl;
L² is selected from the group consisting of *—C(═O)N(R)(optionally substituted C₁-C₆ alkylenyl)-, *—C(═O)O(optionally substituted C₁-C₆ alkylenyl)-, *—C(═O)(optionally substituted C₁-C₆ alkylenyl)-, *-(optionally substituted C₁-C₆ alkylenyl)-, *—N(R⁵)C(═O)(optionally substituted C₁-C₆ alkylenyl)-, *—OC(═O)(optionally substituted C₁-C₆ alkylenyl)-, *—C(═O)N(R⁵)(optionally substituted C₁-C₆ heteroalkylenyl)-, *—C(═O)O(optionally substituted C₁-C₆ heteroalkylenyl)-, *—C(═O)(optionally substituted C₁-C₆ heteroalkylenyl)-, *-(optionally substituted C₁-C₆ heteroalkylenyl)-, *—N(R⁵)C(═O)(optionally substituted C₁-C₆ heteroalkylenyl)-, and *—OC(═O)(optionally substituted C₁-C₆ heteroalkylenyl)-;
R⁴ is an optionally substituted C₆-C₈ heterocycloalkyl,
   wherein the C₆-C₈ heterocycloalkyl comprises at least three atoms independently selected from the group consisting of N, O, and S; and
each occurrence of R⁵ is independently selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

In certain embodiments, the compound, or a salt, solvate, stereoisomer, or isotopologue thereof is selected from the group consisting of:

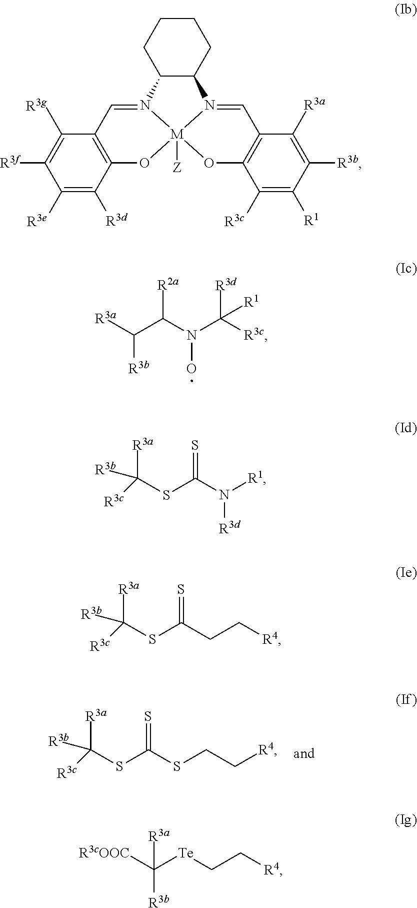

wherein:
M, if present, is Co;
Z is absent or an anionic ligand;

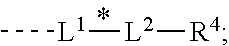

R¹, if present, is
R^{2a}, R^{2b}, and R^{2c}, if present, are each independently selected from the group consisting of H, optionally substituted C₁-C₆ alkyl, optionally substituted C₃-C₈ cycloalkyl, optionally substituted C₆-C₁₀ aryl and optionally substituted C₂-C₁₀ heteroaryl;
R^{3a}, R^{3b}, R^{3c}, R^{3d}, R^{3e}, R^{3f}, R^{3g}, and R^{3h}, if present, are each independently selected from the group consisting of H and optionally substituted C₁-C₆ alkyl;
L¹, if present, is selected from the group consisting of -(optionally substituted C₆-C₁₀ arylenyl)-* and -(optionally substituted C₂-C₁₀ heteroarylenyl)-*.
   wherein a substituent of the C₆-C₁₀ arylenyl or the C₂-C₁₀ heteroarylenyl in L¹ can combine with a substituent of the C₆-C₁₀ aryl or C₂-C₁₀ heteroaryl in R^{2b} to form an optionally substituted C₂₀-C₃₀ heterocycloalkenyl;
L², if present, is selected from the group consisting of *—C(═O)N(R⁵)(optionally substituted C₁-C₆ alkylenyl)-, *—C(═O)O(optionally substituted C₁-C₆ alkylenyl)-, *—C(═O)(optionally substituted C₁-C₆ alkylenyl)-, *-(optionally substituted C₁-C₆ alkylenyl)-, *—N(R⁵)C(═O)(optionally substituted C₁-C₆ alkylenyl)-, *—OC(═O)(optionally substituted C₁-C₆ alkylenyl)-, *—C(═O)N(R⁵)(optionally substituted C₁-C₆ heteroalkylenyl)-, *—C(═O)O(optionally substituted C₁-C₆ heteroalkylenyl)-, *—C(═O)(optionally substituted C₁-C₆ heteroalkylenyl)-, *-(optionally substituted C₁-C₆ heteroalkylenyl)-, *—N(R⁵)C(═O)(optionally substituted C₁-C₆ heteroalkylenyl)-, and *—OC(═O)(optionally substituted C₁-C₆ heteroalkylenyl)-;
R⁴ is an optionally substituted C₆-C₈ heterocycloalkyl,
   wherein the C₆-C₈ heterocycloalkyl comprises at least three atoms selected from the group consisting of N, O, and S; and
      each occurrence of R⁵, if present, is independently selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

In certain embodiments, L¹ is selected from the group consisting of:

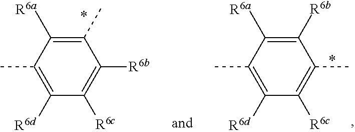

wherein:
R^{6a}, R^{6b}, R^{6c}, and R^{6d} are each independently selected from the group consisting of H, optionally substituted C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵.

In certain embodiments, at least one of R^{6a}, R^{6b}, R^{6c}, and R^{6d} is H. In certain embodiments, at least two of R^{6a}, R^{6b}, R^{6c}, and R^{6d} are H. In certain embodiments, at least three of R^{6a}, R^{6b}, R^{6c}, and R^{6d} are H. In certain embodiments, each of R^{6a}, R^{6b}, R^{6c}, and R^{6d} are H.

In certain embodiments, L² is *—C(═O)NH(C₁-C₆ alkylenyl)-.

In certain embodiments, L² is *—C(═O)NHCH₂CH₂—.

In certain embodiments, R⁴ is:

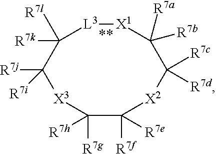

wherein:
L³ is selected from the group consisting of a bond and —X⁴—C(R^{7m})(R⁷ⁿ)—C(R^{7o})(R^{7p})—X¹, X², X³, and X⁴, if present, are each independently selected from the group consisting of N(R⁸) and O;
R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, if present, are each independently selected from the group consisting of H, C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵,
   wherein one of R^{7a}, R^{7b}, R^{7c}, R^{7d}, R^{7e}, R^{7f}, R^{7g}, R^{7h}, R⁷ⁱ, R^{7j}, R^{7k}, and R^{7l} is L²-* or
   one of X¹, X², and X³ is N-L²-*; and
R⁸ is selected from the group consisting of H and optionally substituted C₁-C₆ alkyl.

In certain embodiments, X¹ is N-L²-*. In certain embodiments, X² is N-L²-*. In certain embodiments, X³ is N-L²-*.

In certain embodiments, at least one of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, is H. In certain embodiments, at least two of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least three of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least four of R^{7a}, R^{7b}, R^{7c}, R^{7d}, R^{7e}, R^{7f}, R^{7g}, R^{7h}, R⁷ⁱ, R^{7j}. R^{7k}, R^{7l}, R^{7m}, R⁷ⁿ, R^{7o}, and R^{7p}, are H. In certain embodiments, at least five of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least six of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least seven of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least eight of R^{7a}, R^{7b}, R^{7c}, R^{7d}. R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least nine of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least ten of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least eleven of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least twelve of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least thirteen of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least fourteen of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, at least fifteen of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H. In certain embodiments, each of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p} are H.

In certain embodiments, R⁴ is

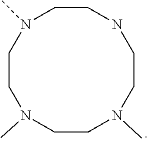

In certain embodiments. R⁴ is

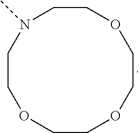

In certain embodiments, R⁴ is and

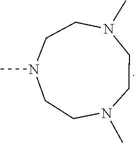

In certain embodiments, R^{2a}, R^{2b}, and R^{2d} are each independently:

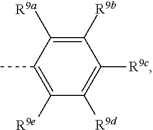

wherein R^{9a}, R^{9b}, R^{7c}, R^{9d}, and R^{9e} are each independently selected from the group consisting of H, optionally substituted C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵.

In certain embodiments, R^{9a} is H. In certain embodiments, R^{9a} is CH₃. In certain embodiments, R^{9b} is H. In certain embodiments, R^{9b} is CH₃. In certain embodiments, R^{9c} is H. In certain embodiments, R^{9c} is CH₃. In certain embodiments, R^{9d} is H. In certain embodiments, R^{9d} is CH₃. In certain embodiments, R^{9e} is H. In certain embodiments, R^{9e} is CH₃.

In certain embodiments, R^{2a} is

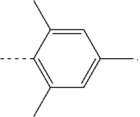

In certain embodiments, R^{2b} is

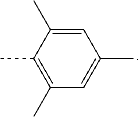

In certain embodiments, R^{2c} is

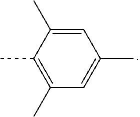

In certain embodiments, at least one of R^{3a}, R^{3b}, R^{3c}, R^{3d}, R^{3e}, R^{3f}, R^{3g}, and R^{3h} is H. In certain embodiments, at least two of R^{3a}, R^{3b}, R^{3c}, R^{3d}, R^{3e}, R^{3f}, R^{3g}, and R^{3h} are H. In certain embodiments, at least three of R^{3a}, R^{3b}, R^{3c}, R^{3d}, R^{3e}, R^{3f}, R^{3g}, and R^{3h} are H. In certain embodiments, at least four of R^{3a}, R^{3b}, R^{3c}, R^{3d}, R^{3e}, R^{3f}, R^{3g}, and R^{3h} are H. In certain embodiments, at least five of R^{3a}, R^{3b}, R^{3c}, R^{3d}, R^{3e}, R^{3f}, R^{3g}, and R^{3h} are H. In certain embodiments, at least six of R^{3a}, R^{3b}, R^{3c}, R^{3d}, R^{3e}, R^{3f}, R^{3g}, and R^{3h} are H. In certain embodiments, at least seven of R^{3a}, R^{3b}, R^{3c}, R^{3d}, R^{3e}, R^{3f}, R^{3g}, and R^{3h} are H. In certain embodiments, each of R^{3a}, R^{3b}, R^{3c}, R^{3d}, R^{3e}, R^{3f}, R^{3g}, and R^{3h} are H.

In certain embodiments, Z is C(═O)OR⁵. In certain embodiments, Z is optionally substituted alkyl. In certain embodiments, Z is halogen. In certain embodiments, Z is C(═O)OMe.

In certain embodiments, the compound is

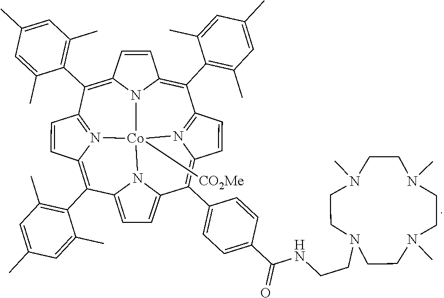

In certain embodiments, the compound is

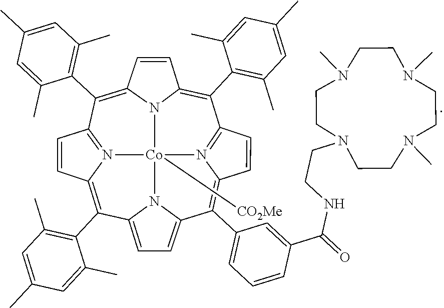

In certain embodiments, the compound is

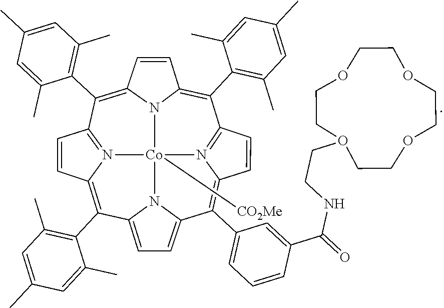

In certain embodiments, the compound is

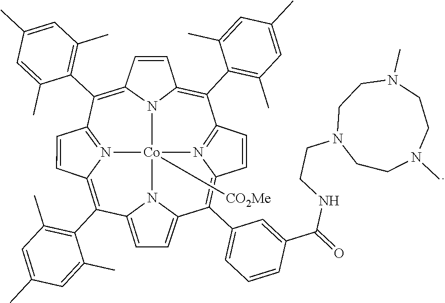

In certain embodiments, the compound of Formula (Ib) is present in an amount selected from the group consisting of about 0.01, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.50, 2.00, 2.50, 3.00, 3.50, 4.00, 4.50, 5.00, 5.50, 6.00, 6.50, 7.00, 7.50, 8.00, 8.50, 9.00, 9.50, 10.00, 10.50, 11.00, 11.50, 12.00, 12.50, 13.00, 13.50, 14.00, 14.50, 15.00, 15.50, 16.00, 16.50, 17.00, 17.50, 18.00, 18.50, 19.00, 19.50, and about 20.0 mol % with respect to the at least two vinyl monomers.

In certain embodiments, the Lewis acid comprises a rare earth metal.

In certain embodiments, the rare earth metal is La(III).

In certain embodiments, the Lewis acid is La(OTf)₃.

In certain embodiments, the Lewis acid is present in an amount selected from the group consisting of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and about 10.0 mol % with respect to the at least two vinyl monomers.

In certain embodiments, the reaction is performed in the presence of a solvent. In certain embodiments, the solvent is aqueous. In certain embodiments, the solvent comprises methanol.

In certain embodiments, the reaction is performed at about room temperature.

In certain embodiments, each vinyl monomer is a compound of Formula (II), which is selected from the group consisting of:

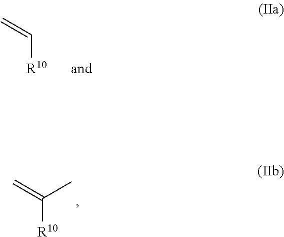

wherein:
R¹⁰ is selected from the group consisting of C(═O)N(R^{11a})(R^{11b}), C(═O)OR^{11a}, OC(═O)R^{11a}, optionally substituted C₂-C₁₀ heteroaryl, OR^{11a}, and CN;
R^{11a} and R^{11b}, if present, are independently selected from the group consisting of H, optionally substituted C₁-C₁ alkyl, optionally substituted C₁-C₁₂ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or
   wherein R^{11a} and R^{11b} can combine with the nitrogen atom to which they are bound to form an optionally substituted C₂-C₈ heterocycloalkyl or C₂-C₁₀ heteroaryl.

In certain embodiments, R^{11a} is C₁-C₆ alkyl. In certain embodiments, R^{11a} is C₁-C₆ hydroxyalkyl. In certain embodiments, R^{11a} is C₁-C₁₂ alkoxyalkyl. In certain embodiments, R^{11a} is C₁-C₆ aminoalkyl.

In certain embodiments, R^{11b} is C₁-C₆ alkyl. In certain embodiments, R^{11a} is C₁-C₆ hydroxyalkyl. In certain embodiments, R^{11b} is C₁-C₁₂ alkoxyalkyl. In certain embodiments, R^{11b} is C₁-C₆ aminoalkyl.

In certain embodiments, R^{11a} is methyl. In certain embodiments, R^{11a} is ethyl. In certain embodiments, R^{11a} is isopropyl. In certain embodiments, R^{11a} is —CH₂CH₂OH. In certain embodiments, R^{11a} is —(CH₂CH₂O)₂₋₃CH₃. In certain embodiments, R^{11a} is —CH₂CH₂CH₂OCH₃. In certain embodiments, R^{11a} is —CH₂CH₂CH₂N(CH₃)₂. In certain embodiments, R^{11a} is —CH₂CH₂CH₂N(CH₃)₃+.

In certain embodiments the vinyl monomer is

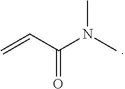

In certain embodiments, the vinyl monomer is

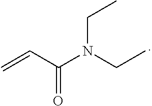

In certain embodiments, the vinyl monomer is

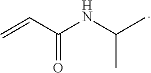

In certain embodiments, the vinyl monomer is

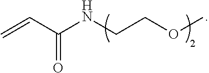

In certain embodiments, the vinyl monomer is

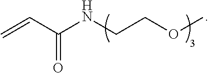

In certain embodiments, the vinyl monomer is

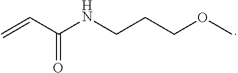

In certain embodiments, the vinyl monomer is

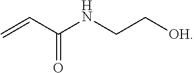

In certain embodiments, the vinyl monomer is

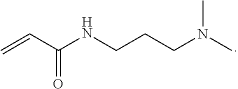

In certain embodiments, the vinyl monomer is

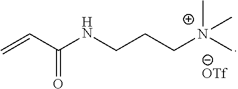

In certain embodiments, the vinyl monomer is

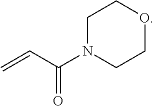

In certain embodiments, the method further comprises terminating the polymerization reaction to obtain a polymer product.

In certain embodiments, the polymer product comprises at least one selected from the group consisting of

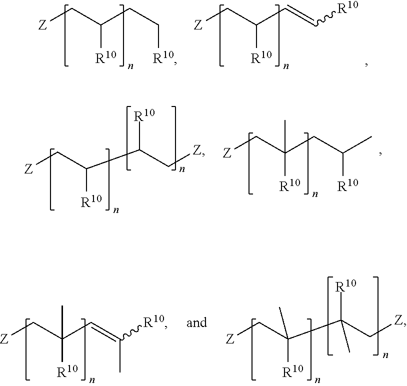

wherein:
R¹⁰ is selected from the group consisting of C(═O)N(R^{11a})(R^{11b}) C(═O)OR^{11a} and CN;
R^{11a} and R^{11b}, if present, are independently selected from the group consisting of H, optionally substituted C₁-C₁₂ alkyl, optionally substituted C₁-C₁₂ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or
   wherein R^{11a} and R^{11b} can combine with the nitrogen atom to which they are bound to form an optionally substituted C₂-C₈ heterocycloalkyl or C₂-C₁₀ heteroaryl; and
each occurrence of n is independent an integer from 1 to 5,000.

In certain embodiments, R¹⁰ is

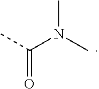

In certain embodiments, R¹⁰ is

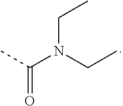

In certain embodiments, R¹⁰ is

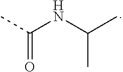

In certain embodiments, R¹⁰ is

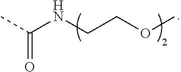

In certain embodiments, R¹⁰ is

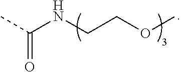

In certain embodiments, R¹⁰ is

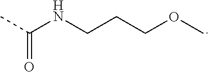

In certain embodiments, R¹⁰ is

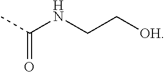

In certain embodiments, R¹⁰ is

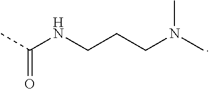

In certain embodiments, R¹⁰ is

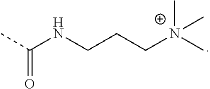

In certain embodiments, R¹⁰ is

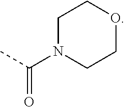

In certain embodiments, the method further comprises (d) contacting the catalyst-nascent polymer complex with at least two second vinyl monomers to provide a second mixture. In certain embodiments, each second vinyl monomer is substituted with at least one substituent comprising a heteroatom having at least one coordinating lone pair of electrons. In certain embodiments, each second vinyl monomer is substituted with identical substituents. In certain embodiments, the first vinyl monomer and second vinyl monomer are not identical. In certain embodiments, the method further comprises (e) irradiating the second mixture to provide a second catalyst-nascent polymer complex.

In certain embodiments, the method further comprises (f) contacting the second catalyst-nascent polymer complex with at least two additional vinyl monomers to provide an additional mixture. In certain embodiments, each second vinyl monomer is substituted with at least one substituent comprising a heteroatom having at least one coordinating lone pair of electrons. In certain embodiments, each additional vinyl monomer is substituted with identical substituents. In certain embodiments, the method further comprises (g) irradiating the additional mixture to provide an additional catalyst-nascent polymer complex.

In certain embodiments, steps (f) and (g) may be repeated with two or more additional vinyl monomers.

In certain embodiments, the method further comprises terminating the reaction to obtain a polymer product.

In certain embodiments, the polymer product has a tacticity selected from the group consisting of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and about 100% m.

In certain embodiments, the tacticity of the polymer product is positively correlated with the amount of the Lewis acid-catalyst complex.

### EXAMPLES

Various embodiments of the present application can be better understood by reference to the following Examples which are offered by way of illustration. The scope of the present application is not limited to the Examples given herein.

### Materials and Methods

### Materials

All chemicals were used as received from Sigma Aldrich, Acros Organics, Alfa Aesar, or TCI unless otherwise specified. ACS grade solvents were used as received from Sigma Aldrich and Macron Fine Chemicals. Commercial adhesives used in the lap shear tests include: washable glue (Slime E305, Elmer's, Ohio, U.S.), double-sided tape (CT220, XFasten, Connecticut, U.S.), and poster strips (17024D, 3M, Minnesota, U.S.). Neutral and basic aluminum oxides (Al₂O₃) (standard activity I grade) were purchased from Sorbent Technologies, Inc. (Sorbtech) with 50-200 μm particle size. Silica gel was purchased from Sorbtech (standard grade) with 60 Å porosity and 40-63 μm particle size. N-(2-(2-methoxyethoxy)ethyl)acrylamide (2f). N-(2-(2-(2-methoxyethoxy)ethoxy)ethyl)acrylamide (2g), and 3-((3-acrylamidopropyl)dimethylammonio)propane-1-sulfonate (2k) were prepared according to methods known to those of ordinary skill in the art. All liquid monomers used in this work were passed through a short column of basic Al₂O₃ to remove inhibitor prior to use. N-isopropylacrylamide (NIPAM) was recrystallized in hexane before use.

### Methods

Analytical thin layer chromatography (TLC) was performed on Merck precoated TLC plates (silica gel 60 GF254, 0.25 mm). Preparative thin layer chromatography (prep-TLC) was performed on Merck precoated TLC plates (silica gel 60 GF254, 0.50 mm). Analytical gel permeation chromatography (GPC) measurements were taken at a concentration of 0.1-1.0 mg/mL in 10 mM LiBr-DMF solution (J. T. Baker or Fisher) on a TOSOH Bioscience EcoSEC HKC08320 GPC with differential refractive index (RI) detector, equipped with a TSKgel GMHHR-M column calibrated with poly(methyl methacrylate) standards. Proton nuclear magnetic resonance (¹H-NMR) spectra were recorded on an Agilent DD₂ 400 MHz spectrometer or an Agilent DD₂ 500 MHz spectrometer with 10 s relaxation time. Chemical shifts are reported in ppm using solvent resonance as the internal standard (CDCl₃: 7.26 ppm; DMSO-d₆: 2.50 ppm; CD₃COD: 3.31 ppm). Data are reported as follows: chemical shift, multiplicity (s=singlet, d=doublet, t=triplet, q=quartet, m=multiplet, dd=doublet of doublets, ddd=doublet of doublet of doublets), coupling constants (Hz), and integration. ¹³C-NMR spectra were recorded on an Agilent DD₂ 400 MHz spectrometer (101 MHz) or an Agilent DD₂ 500 MHz spectrometer (150 MHz) with complete proton decoupling. Chemical shifts are reported in ppm from the solvent resonance as the internal standard (CDCl₃: 77.0 ppm; DMSO-d₆: 2.50 ppm; CD₃COD: 44.8 ppm). CDCl₃, DMSO-d₆, and CD₃COD were purchased from Cambridge Isotope Laboratories, Inc. NMR spectra were processed using MestReNova 10.0.1. High-resolution mass spectra (HRMS) were obtained from a Shimadzu 9030 Quadrupole mass spectrometer. Density functional theory (DFT) calculations were performed with the Gaussian 09 and Gaussian View 16 package. Light intensity applied in photopolymerization was determined by Ophir power meter P/N7Z02621 (3A).

Differential scanning calorimetry (DSC) was used to evaluate the thermal properties of the synthesized samples, including the crystallization temperature (T_{c}), melting point (Tₘ), and glass-transition temperature (T_{g}) on a TA Instruments Discovery type calorimeter. Tₘ and T_{g} values were typically obtained from a second heating scan after the thermal history was removed. All heating and cooling rates were 5° C./min. Decomposition onset temperatures (Td) and maximum decomposition temperatures (Tₘₐₓ) of precipitated and dried polymer samples were measured by thermal gravimetric analysis (TGA) on a TA Instruments Q50 thermogravimetric analyzer. Polymer samples were heated from ambient temperatures to 800° C. at a heating rate of 10° C./min. Values of Td (temperature at 5% weight loss) were obtained from percent of weight (wt %) vs temperature plots, and values of Tₘₐₓ were obtained from plots of mass loss derivative (wt %/° C.) vs temperature. All samples were annealed at 80° C. for 15 hours before tested on DSC and TGA.

Wide angel X-ray scattering (WAXS) samples were loaded into the center of washers acting as sample holders. Transmission WAXS was conducted on an in-house X-ray scattering instrument (Xenocs Xeuss 3.0). The sample to detector distance was 900 mm and the wavelength (λ) of the beam was 1.54 Å (8.05 keV). The 2-D scattering patterns were azimuthally integrated into 1-D plots of scattering intensity (1) versus scattering vector q, where q=47 sin 0/λ and the scattering angle is 20. Samples were annealed at 190° C. for 3 days before test.

Lower critical solution temperatures (LCST) of polyDEAA were measured by a Shimadzu UV-3600Plus or a Unisoku UV-Vis spectrometer scanned with a programmed temperature profile. Samples were dissolved in H₂O at a concentration of 5 mg/mL for all measurements. During each measurement, transmittance of polymer solution was recorded after isothermal equilibration at each temperature for five minutes. The LCST was determined to be the temperature at which 50% transmittance dropped.

Lap shear measurement was conducted using an Instron 5960 tensile testing system. Lap shear stress and strain were recorded at room temperature at an extension speed of 5.0 mm/min. Purified polyHEAA (1 g) was first dissolved in methanol (10 mL), followed by addition of glycerol (1 g). The mixtures were vigorously stirred until homogenous solutions formed. Methanol was removed under reduced pressure and the resulting mixtures were employed for the measurement. Glass slides (8.5 cm×2.2 cm×1.5 mm) were directly used without any pre-treatment. Both polyHEAA and commercial samples were painted on a 2.2 cm×2.0 cm area with 0.1 mm in thickness. The adhered interfaces were equilibrated at room temperature for ca. 3 hours before testing.

EIS measurements were performed using a Solartron SI 1260 impedance/gain phase analyzer by sandwiching polymer electrolyte films in a symmetric stainless steel (SS∥SS) coin cell. A frequency range of 10⁶ Hz to 1 Hz with a polarization amplitude of 20 mV was used. All samples were fabricated and tested in a nitrogen glovebox. Temperature-dependent ionic conductivity was measured from 25 to 80° C. with 30-minute equilibration time at each temperature. Polymers were dissolved in acetonitrile (˜50 mg/mL), stirred for approximately 3 hours, and drop-casted onto circular stainless-steel discs in the glovebox. A Teflon ring with a thickness of 0.01 inch and an inner diameter of ⅜ inch was used as a spacer to ensure no thickness variation during the measurements. Acetonitrile was slowly evaporated in the glovebox for over 8 hours, which produced smooth and homogenous films. The samples were then placed into the vacuum chamber of the glovebox and dried under vacuum for 12 hours.

### Example 1: Chemical Synthesis

### Synthesis of L1

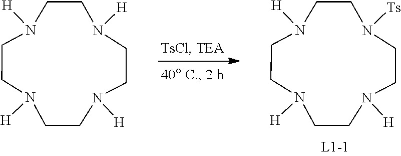 [SMILES: C1CNCCNCCNCCN1 ; Cc1ccc(S(=O)(=O)N2CCNCCNCCNCC2)cc1]

### 1-tosyl-1,4,7,10-tetraazacyclododecane (L1-1)

Cyclen (4.75 g, 27.6 mmol) and triethylamine (TEA) (10 mL) were dissolved in 100 mL anhydrous chloroform (CHCl₃) at 0° C. Then, p-toluene chloride (5 g, 26.2 mmol) dissolved in 10 mL anhydrous CHCl₃ was slowly added to the solution. The mixture was stirred at 40° C. for 2 hours, and another 16 hours at ambient temperature under N₂ atmosphere. The reaction mixture was washed with water (4×50 mL). All volatiles were removed under reduced pressure. Water was added to the residue and the supernatant was removed after the suspension was stirred for 3 h at ambient temperature. More water was added to the residue, and the supernatant was removed after another stirring for 3 h. The combined aqueous layers were extracted with CHCl₃ (3×30 mL). Removal of CHCl₃ under reduced pressure gave 7.0 g of 1-tosyl-1,4,7,10-tetraazacyclododecane in 85% yield. Spectral data for 1-tosyl-1,4,7,10-tetraazacyclododecane (L1-1): ¹H-NMR (400 MHz, CDCl₃): δ 7.66 (d, J=8.0 Hz, 2H), 7.28 (d, J=8.0 Hz, 2H), 3.18 (t, J=5.2 Hz, 4H), 2.76-72.74 (m, 8H), 2.63-2.61 (m, 4H), 2.39 (s, 3H), The spectral data for this compound match that reported in the literature.

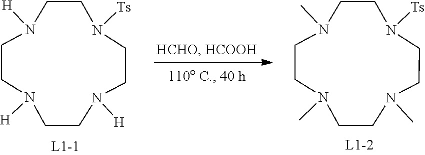 [SMILES: Cc1ccc(S(=O)(=O)N2CCNCCNCCNCC2)cc1 ; Cc1ccc(S(=O)(=O)N2CCN(C)CCN(C)CCN(C)CC2)cc1]

### 1,4,7-trimethyl-10-tosyl-1,4,7,10-tetraazacyclododecane (L1-2)

L1-1 (7.0 g) was dissolved in 99% formic acid (20 mL) and 37% formaldehyde solution (7 mL). The mixture was stirred at 110° C. for 40 h. Sodium hydroxide was added to the reaction mixture at 0° C. until pH >12. The aqueous phase was extracted with CHCl₃ (3×20 mL). Organic solution was dried over anhydrous Na₂SO₄ and evaporated to provide 4.9 g (70% yield) of 1,4,7-trimethyl-10-tosyl-1,4,7,10-tetraazacyclododecane (L1-2). ¹H-NMR (400 MHz, CDCl₃) δ: 7.53 (d, 2H, J=8.1 Hz), 7.14 (d, 2H, J=8.1 Hz), 3.10 (t, 4H, J=6.2 Hz), 2.65 (t, 4H, J=6.2 Hz), 2.36-2.32 (m, 8H), 2.26 (s, 3H), 2.11 (s, 6H), 2.07 (s, 3H). The spectral data for this compound match that reported in the literature.

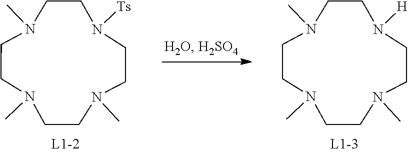 [SMILES: Cc1ccc(S(=O)(=O)N2CCN(C)CCN(C)CCN(C)CC2)cc1 ; CN1CCNCCN(C)CCN(C)CC1]

### 1,4,7-trimethyl-1,4,7,10-tetraazacyclododecane (L1-3)

L1-2 (4.9 g) and 18 mol/L sulfuric acid (15 mL) were mixed and stirred at 100° C. for 3 days. After cooling down to 0° C., sodium hydroxide was added to the reaction mixture until pH >12. The aqueous phase was extracted with CHCl₃ (4×100 mL). The combined CHCl₃ layers were dried with MgSO₄. The solvent was removed under reduced pressure, and the residue was purified using vacuum distillation (0.1-0.2 mmHg, 110-120° C.), giving 2.2 g of 1,4,7-trimethyl-1,4,7,10-tetraazacyclododecane (L1-3) (67% yield) as a colorless viscous oil. Spectral data: ¹H-NMR (400 MHz, CDCl₃): δ 2.64 (t, J=5.2 Hz, 4H), 2.45 (t, J=5.2 Hz, 4H), 2.39 (s, 8H), 2.28 (s, 6H), 2.24 (s, 3H). The spectral data for this compound match that reported in the literature.

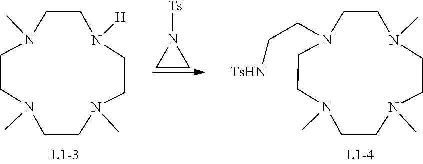

### 4-methyl-N-(2-(4,7,10-trimethyl-1,4,7,10-tetraazacyclododecan-1-yl)ethyl)benzenesulfonamide (L1-4)

(L1-3) (500 mg) was dissolved in 50 mL of toluene. 1-Tosylaziridine (460 mg) in 7 mL of toluene was dropwise added into the solution within 2 hours under N₂ atmosphere. The mixture was stirred for another 12 hours. The reaction mixture was washed with water (4×50 mL) to remove unreacted 1-tosylaziridine. The solvent in the collected organic layer was removed under reduced pressure to give 930 mg (97% yield) of L1-4. Spectral data (L1-4): ¹H-NMR (400 MHz, CDCl₃) δ: 7.53 (d, 2H, J=8.1 Hz), 7.14 (d, 2H, J=8.1 Hz), 3.10 (t, 4H, J=6.2 Hz), 2.65 (t, 4H, J=6.2 Hz), 2.32-2.36 (m, 8H), 2.26 (s, 3H). 2.11 (s, 6H), 2.07 (s, 3H). ¹³C-NMR (101 MHz, CDCl₃) δ: 142.8, 135.8, 129.4, 126.9, 56.0, 55.8, 55.4, 53.7, 53.5, 45.3, 43.4, 43.1, 21.4. HRMS (ESI) calculated for C₂₀H₃₈N₅SO₂ [M+H⁺]: 412.2741. found: 412.2738.

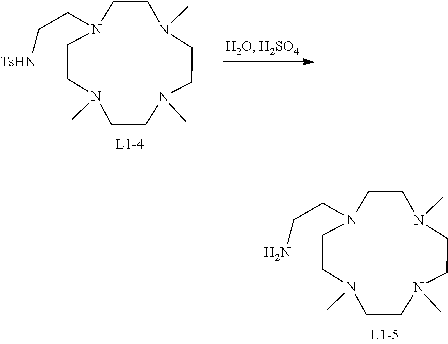

### 2-(4,7,10-trimethyl-1,4,7,10-tetraazacyclododecan-1-yl)ethan-1-amine (L1-5)

L1-4 (930 mg) and 18 mol/L sulfuric acid (5 mL) were mixed and stirred at 100° C. for 3 days. After cooling down to 0° C., sodium hydroxide was added to the reaction mixture until pH >12. The aqueous phase was extracted with CHCl₃ (4×100 mL). The combined chloroform layers were dried with MgSO₄, the solvent was removed under reduced pressure, giving 280 mg of L1-5 (58% yield) as a colorless viscous oil. Spectral data (L1-5): ¹H-NMR (401 MHz, CDCl₃): δ 2.62-2.60 (m, 2H), 2.45-2.05 (m, 27H)¹³C-NMR (101 MHz, CDCl₃): δ 55.9, 55.6, 54.8, 54.3, 52.3, 51.4, 44.7, 43.9. HRMS (ESI) calculated for C₁₃H₃₁N₅[M+H⁺]: 257.2653, found: 257.2657.

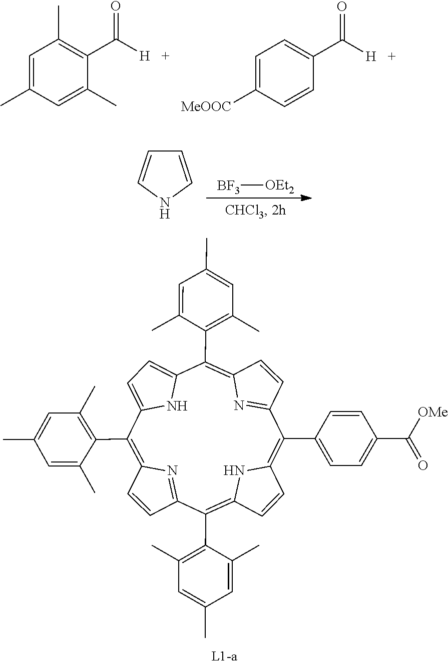

### 5-(4-methoxycarbonylphenyl)-10,15,20-tris(2,4,6-trimethylphenyl)porphyrin (L1-a)

Methyl 4-formylbenzoate (1.3 g. 8 mmol), pyrrole (2.2 mL, 32 mmol), and BF₃OEt₂ (1.3 mL) were added to a CHCl₃ (800 mL) solution of 2,4,6-trimethylbenzaldehyde (3.6 g, 24 mmol). The solution was stirred at room temperature for 2 h. Then, p-chloranil (5.0 g) was added and stirred at room temperature for 1 h. After TEA (1.0 mL) was added, the solvent was removed in vacuo. Silica gel based column chromatography (CH₂Cl₂/hexane=1/1 v/v) afforded L1-a as a purple solid (1.0 g. 1.3 mmol, 16% yield). ¹H-NMR (400 Hz, CDCl₃): δ 8.73-8.43 (s, 8H), 8.42 (d, J=8.4 Hz, 2H), 8.32 (d, J=8.0 Hz, 2H), 7.30 (s, 6H), 4.08 (s, 3H), 2.61 (s, 9H), 1.86 (s, 18H). The spectral data for this compound match that reported in the literature.

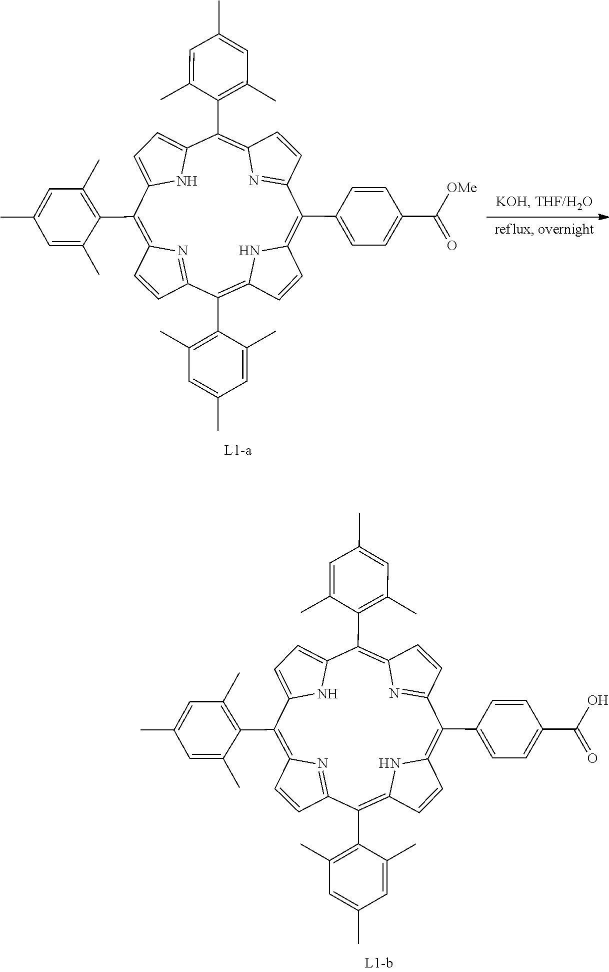 [SMILES: COC(=O)c1ccc(-c2c3nc(c(-c4c(C)cc(C)cc4C)c4ccc([nH]4)c(-c4c(C)cc(C)cc4C)c4nc(c(-c5c(C)cc(C)cc5C)c5ccc2[nH]5)C=C4)C=C3)cc1 ; Cc1cc(C)c(-c2c3nc(c(-c4c(C)cc(C)cc4C)c4ccc([nH]4)c(-c4c(C)cc(C)cc4C)c4nc(c(-c5ccc(C(=O)O)cc5)c5ccc2[nH]5)C=C4)C=C3)c(C)c1]

### 5-(4-Carboxyphenyl)-10,15,20-tris(2,4,6-trimethylphenyl)porphyrin (L1-b)

A solution of L1-a (1.0 g, 1.3 mmol) in tetrahydrofuran (THF) (100 mL) was added with a solution of KOH (2.5 g) in H₂O (50 mL). The solution was refluxed for 2 h. After the reaction mixture was cooled to room temperature, aqueous 1 M HCl (10 mL) was added. The solution was washed with saturated aqueous NaHCO₃ (50 mL) and H₂O (50 mL×3), and dried over anhydrous Na₂SO₄. The solvent was removed in vacuo. Column chromatography on silica gel (CH₂Cl₂/MeOH=10/1 v/v) gave L1-b as a purple solid (0.92 g, 1.2 mmol, 92% yield). ¹H-NMR (400 Hz, CDCl₃): δ 8.78 (s, 2H), 8.63 (s, 6H), 8.42 (d, J=8.4 Hz, 2H), 8.28 (d, J=8.4 Hz, 2H), 7.29 (s, 2H), 7.28 (s, 4H), 2.61 (s, 3H), 2.60 (s, 6H), 1.86 (s, 6H), 1.83 (s, 12H). The spectral data for this compound match that reported in the literature.

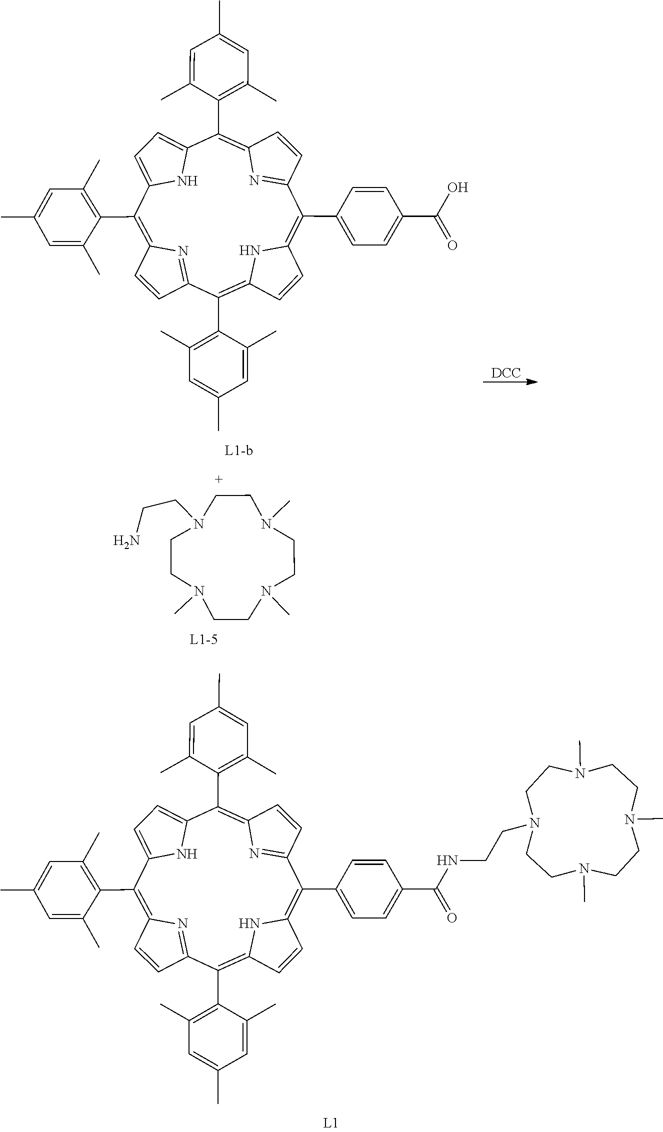

### 2-(4,7,10-trimethyl-1,4,7,10-tetraazacyclododecan-1-yl)ethyl4-(10,15,20-trimesitylporphyrin-5-yl)benzoate (L1)

L1-b (200 mg) was dissolved in 10 mL anhydrous CH₂Cl₂, then N-hydroxysuccinimide (NHS) (68 mg) and N,N′-dicyclohexylcarbodiimide (DCC) (60 mg) were add. The solution was heated to 40° C. and stirred for 24 hours, followed up by 200 mg L1-5 and another 24 hours under 40° C. The solution was washed with saturated aqueous Na₂CO₃ (50 mL) and H₂O (50 mL×3) and dried over anhydrous Na₂SO₄, and then the solvent was removed in vacuo. The product was purified (119 mg, 47% yield) by prep-TLC (CH₂Cl₂/MeOH/TEA=10/1/0.1 v/v/v, retention factor R_{f}=0.2). ¹H-NMR (400 Hz, CDCl₃): δ 8.75-8.68 (m, 8H), 8.29-8.26 (m, 4H), 7.28 (s, 6H), 3.70 (t. J=8.2 Hz, 2H), 2.74-2.56 (m, 33H), 2.23 (s, 6H), 2.12 (s, 3H), 1.86 (s, 18H). ¹³C-NMR (101 MHz, CDCl₃): δ 167.6, 139.5, 139.2, 138.2, 137.8, 137.1, 127.8, 127.0, 118.1, 117.9, 70.6, 54.5, 48.9, 42.4, 34.0, 25.6, 21.8, 21.7, 21.5. HRMS (ESI) calculated for C₆₇H₇₈N₉O[M+H⁺]: 1024.6324, found: 1024.6325.

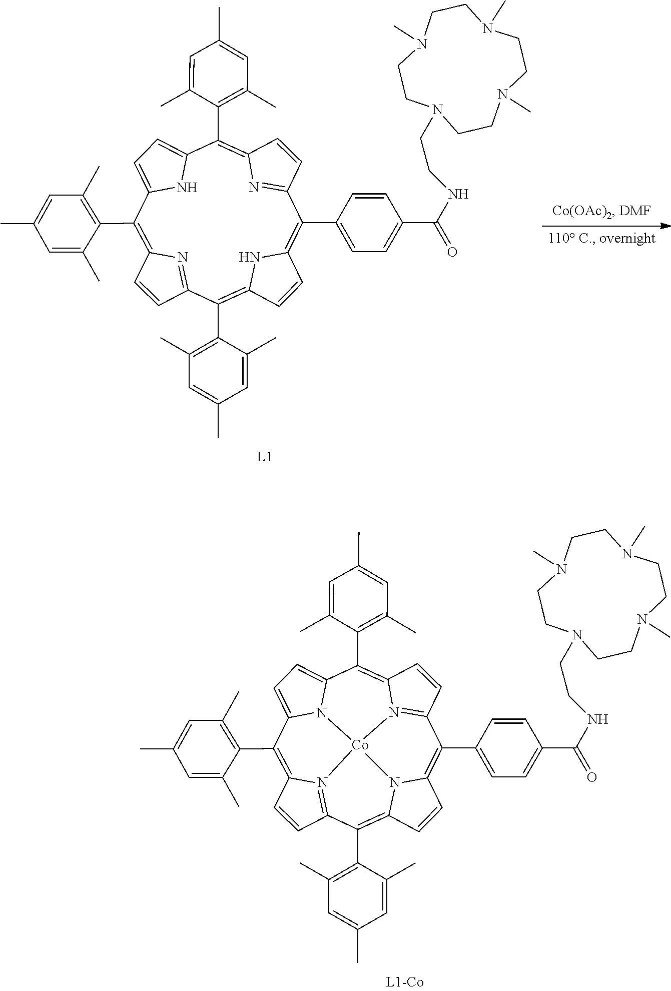

### Synthesis of L1-Co

L1 (100 mg) was dissolved in 10 mL of DMF. 80 mg of Co(OAc)₂ was added. The mixture was stirred at 150° C. for 12 h. After the reaction mixture was cooled to room temperature, 40 mL of CH₂C₂ was added. The solution was washed with saturated aqueous NaHCO₃ (50 mL) and H₂O (50 mL×3), dried over anhydrous sodium sulfate, and then the solvent was removed in vacuo. NMR measurement was not accessible because cobalt/porphyrin is a paramagnetic compound. HRMS (ESI) calculated for C₆₇H₇₆N₉OCo[M+H⁺]: 1081.5500, found: 1081.5501.

### Synthesis of L2

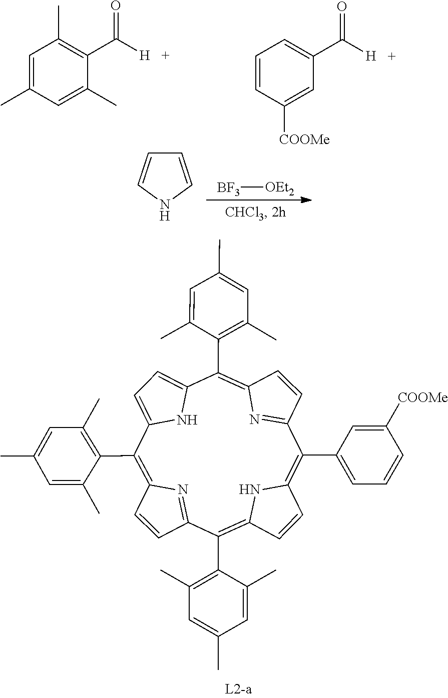

### 5-(3-methoxycarbonylphenyl)-10,15,20-tris(2,4,6-trimethylphenyl)porphyrin (L2-a)

To a solution of 2,4,6-trimethylbenzaldehyde (3.6 g, 24 mmol) in CHCl₃ (800 mL), methyl 3-formylbenzoate (1.3 g, 8 mmol), pyrrole (2.2 mL, 32 mmol), and BF₃OEt₂ (1.3 mL) were added. The solution was stirred at room temperature for 2 h. Then, p-chloranil (5.0 g) was added and stirred at room temperature for 1 h. After Et₃N (1.0 mL) was added, the solvent was removed in vacuo. Column chromatography on silica gel (CH₂Cl₂/hexane=1/1 v/v) afforded L2-a as a purple solid (1.0 g, 1.3 mmol, 16% yield). ¹H-NMR (400 Hz, CDCl₃): δ 8.71 (s, 1H), 8.67-8.40 (m, 8H), 8.37 (d, J=8.4 Hz, 1H), 8.37 (t, J=8.8 Hz, 1H), 7.83 (t, J=8.0 Hz, 1H), 7.30 (s, 6H), 4.01 (s, 3H), 2.64 (s, 9H). 1.88 (s, 18H). HRMS (ESI) calculated for C₅₅H₅₁N₄O₂[M+H⁺]: 799.4007, found: 799.4005.

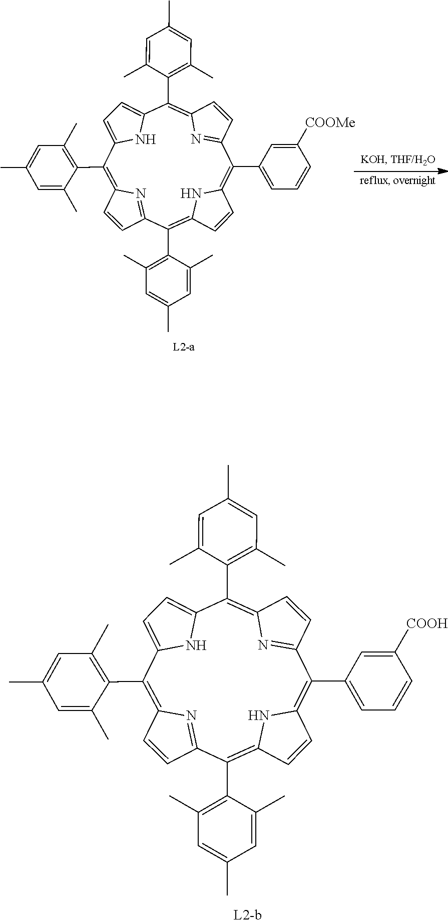 [SMILES: COC(=O)c1cccc(-c2c3nc(c(-c4c(C)cc(C)cc4C)c4ccc([nH]4)c(-c4c(C)cc(C)cc4C)c4nc(c(-c5c(C)cc(C)cc5C)c5ccc2[nH]5)C=C4)C=C3)c1 ; Cc1cc(C)c(-c2c3nc(c(-c4c(C)cc(C)cc4C)c4ccc([nH]4)c(-c4c(C)cc(C)cc4C)c4nc(c(-c5cccc(C(=O)O)c5)c5ccc2[nH]5)C=C4)C=C3)c(C)c1]

### 5-(3-Carboxyphenyl)-10,15,20-tris(2,4,6-trimethylphenyl)porphyrin (L2-b)

A solution of L2-a (1.0 g, 1.3 mmol) in THF (100 mL) was added with a solution of KOH (2.5 g) in H₂O (50 mL). The solution was refluxed for 2 h. After the reaction mixture was cooled to room temperature, aqueous 1 M HCl (10 mL) was added. The solution was washed with saturated aqueous NaHCO₃ solution (50 mL) and H₂O (50 mL×3), dried over by anhydrous Na₂SO₄. The solvent was removed in vacuo. Column chromatography on silica gel (CH₂Cl₂/MeOH=10/1 v/v) gave L2-b as a purple solid (0.92 g, 1.2 mmol, 92% yield). ¹H-NMR (400 Hz, CDCl₃): δ 8.82 (d, J=5.4 Hz, 2H), 8.68 (d, J=5.2 Hz, 2H), 8.42 (s, 4H), 7.79 (d J=8.4 Hz, 1H), 7.66 (d, J=6.4 Hz, 1H), 7.55-7.52 (in. 1H), 7.27 (s, 6H), 2.61 (s, 9H), 1.87 (s, 18H). HRMS (ESI) calculated for C₆₇H₇₈N₉O[M+H⁺]: 785.3851, found: 785.3849.

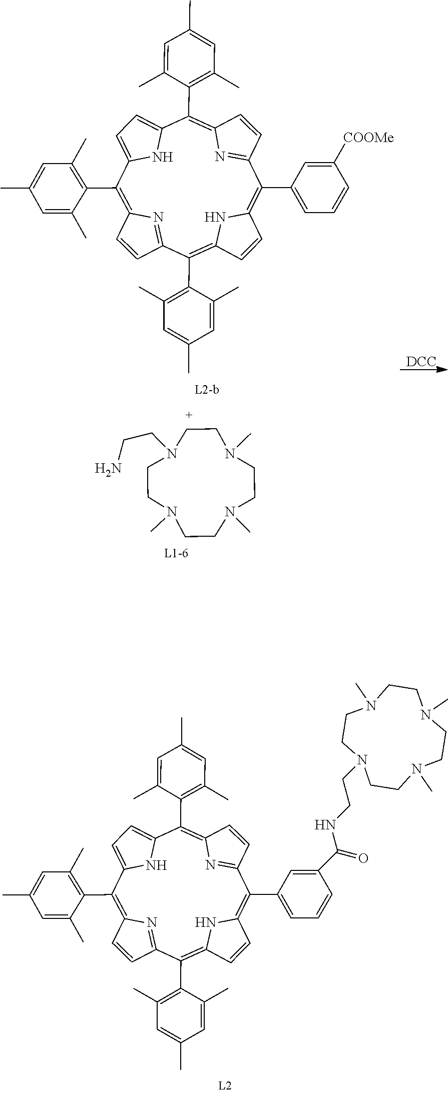

### 2-(4,7,10-trimethyl-1,4,7,10-tetraazacyclododecan-1-yl)ethyl4-(10,15,20-trimesitylporphyrin-5-yl)benzoate (L2)

L2-b (200 mg) was dissolved in 10 mL anhydrous CH₂Cl₂, then NHS (68 mg) and DCC (60 mg) were added. The solution was heated to 40° C. and stirred for 24 h, followed by addition of 200 mg of L1-5 and another 24 h stirring at 40° C. The solution was washed with saturated aqueous Na₂CO₃ solution (50 mL) and H₂O (50 mL×3), and dried over anhydrous Na₂SO₄. The solvent was removed in vacuo. The product was purified by prep-TLC (CH₂Cl₂/MeOH/TEA=10/1/0.1 v/v/v, R_{f}=0.2). ¹H-NMR (400 Hz, CDCl₃): δ 8.66-8.60 (m, 10H), 8.19-8.21 (m, 1H), 7.75-7.76 (m, 1H), 7.20 (s, 6H), 3.58 (t, J=6.9 Hz, 2H), 3.12-3.09 (t, J=6.9 Hz, 2H), 2.55-2.38 (m, 31H), 1.80-1.75 (m, 18H). ¹³C-NMR (101 MHz, CDCl₃): δ 167.3, 141.9, 139.1, 139.0, 138.9, 137.9, 137.7, 137.6, 127.6, 117.9, 117.8, 70.4, 47.8, 55.8, 54.7, 50.0, 37.1, 25.4, 21.6, 21.5, 21.3. HRMS (ESI) calculated for C₆₇H₇₈N₉O[M+H⁺]: 1024.6324. found: 1024.6325.

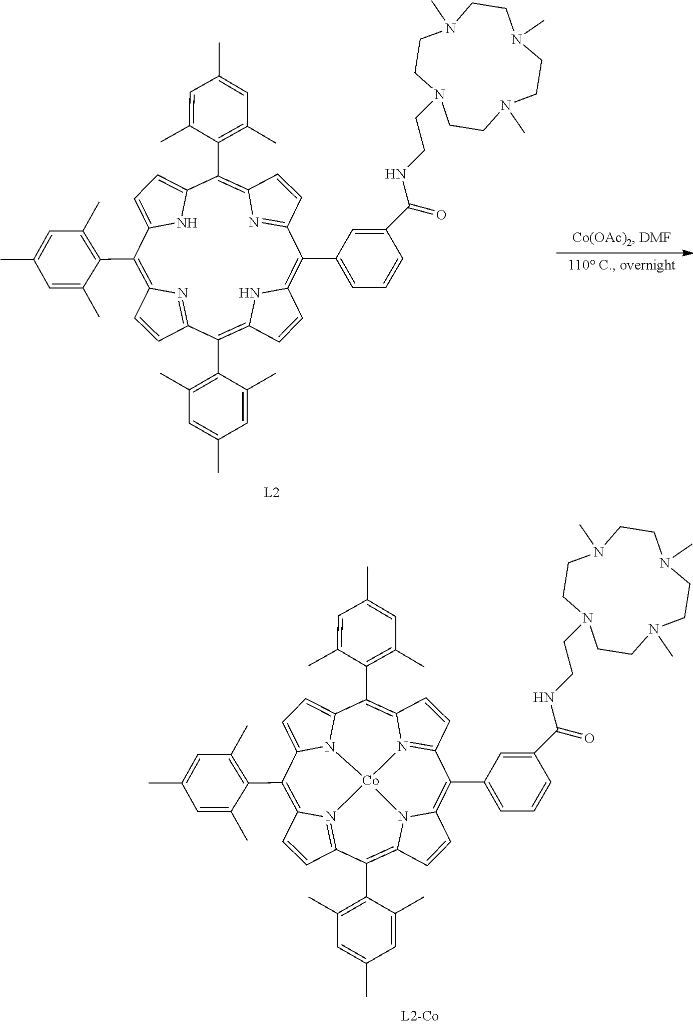

### Synthesis of L2-Co

L2 (100 mg) was dissolved in 10 mL of DMF. 80 mg of Co(OAc)₂ was then added. The mixture was stirred at 150° C. for 12 h. After the reaction mixture was cooled to room temperature, 40 mL of CH₂Cl₂ was added. The solution was washed with saturated aqueous NaHCO₃ solution (50 mL) and H₂O (50 mL×3), and dried over anhydrous Na₂SO₄. The solvent was removed in vacuo. NMR measurement was not accessible because cobalt/porphyrin is a paramagnetic compound. HRMS (ESI) calculated for C₆₇H₇₆N₉OCo[M+H⁺]: 1081.5500, found: 1081.5501.

### Synthesis of L3

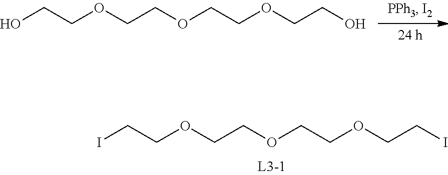 [SMILES: OCCOCCOCCOCCO ; ICCOCCOCCOCCI]

### 1-iodo-2-(2-(2-(2-iodoethoxy)ethoxy)ethoxy)ethane (L3-1)

12 (8.91 g, 35.09 mmol), triphenylphosphine (9.20 g, 35.09 mmol), and imidazole (2.99 g, 43.86 mmol) were dissolved in anhydrous CH₂Cl₂ (90 mL). The mixture was stirred at room temperature for 5 min. A solution of 2,2′-((oxybis(ethane-2,1-diyl))bis(oxy))bis(ethan-1-ol) (2.60 g) in anhydrous CH₂Cl₂ (10 ML) was added to the mixture dropwise. The reaction was stirred at room temperature for another 3 h and quenched with saturated Na₂S₂O₃ solution. The aqueous layer was extracted with CH₂Cl₂ and the extract was combined with the organic layer. The combined organic layers were washed with brine and dried over anhydrous Na₂SO₄, followed by vacuum concentration. The product (91% yield) was obtained by silica gel column chromatography (5% to 20% EtOAc in hexane). ¹H-NMR (400 Hz, CDCl₃): δ 3.77 (t. J=6.9 Hz, 4H), 3.67 (s, 8H), 3.28 (t, J=6.9 Hz, 4H). The spectral data for this compound match that reported in the literature.

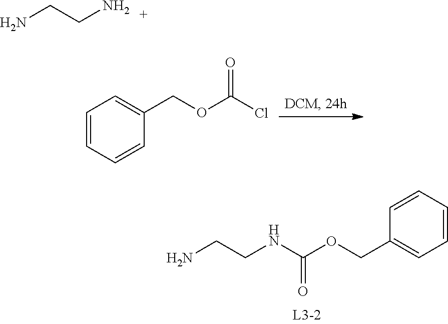

### benzyl (2-aminoethyl)carbamate (L3-2)

A solution of benzylchloroformate (1.3 mL, 9.0 mmol) in dry CH₂Cl₂ (25 mL) was added over 1.5 h to a solution of ethylenediamine (6.0 mL, 90 mmol) in anhydrous CH₂Cl₂ (90 mL) at 0° C. under N₂ atmosphere. The mixture was stirred at 0° C. for 2 h and washed with brine. The organic layer was dried over MgSO₄ and concentrated under reduced pressure. The product (85% yield) was purified by silica gel column chromatography (20% to 800% EtOAc in hexane). H-NMR (400 Hz, CDCl₃): δ 7.35-7.26 (5H, m), 5.06 (2H, s), 3.19 (2H, t, J=6.0 Hz). 2.73 (2H, t, J=6.0 Hz); The spectral data for this compound match that reported in the literature.

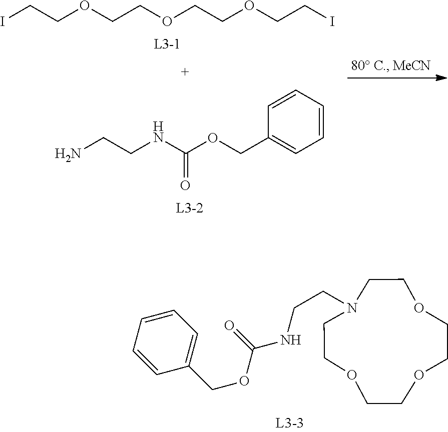 [SMILES: ICCOCCOCCOCCI ; NCCNC(=O)OCc1ccccc1 ; O=C(NCCN1CCOCCOCCOCC1)OCc1ccccc1]

### benzyl (2-(1,4,7-trioxa-10-azacyclododecan-10-yl)ethyl)carbamate (L3-3)

A stirred solution of 1-iodo-2-(2-(2-(2-iodoethoxy)ethoxy)ethoxy)ethane (L3-1) (4.3 g, 0.01 mol) and benzyl (2-aminoethyl)carbamate (L3-2) (0.011 mol) in acetonitrile (MeCN) (150 mL) containing anhydrous Na₂CO₃ (5.3 g, 0.05 mol) was refluxed under an atmosphere of N₂ for 18 h. The mixture was then allowed to cool and filtered. The filtrate was concentrated. The residue was stirred in CH₂Cl₂ (150 mL) and filtered to remove the residual salts. The solvent was removed in vacuo. The pure product was obtained after column chromatography on silica gel (CH₂Cl₂/MeOH/TEA=10/1/0.1 v/v/v). ¹H-NMR (400 Hz. CDCl₃): δ 7.46-7.20 (5H, m), 5.17 (2H, s), 3.70-3.30 (m, 16H), 2.62-2.43 (m, 4H). HRMS (ESI) calculated for C₁₈H₂₉N₂O₅[M+H]: 353.2071, found: 353.2068.

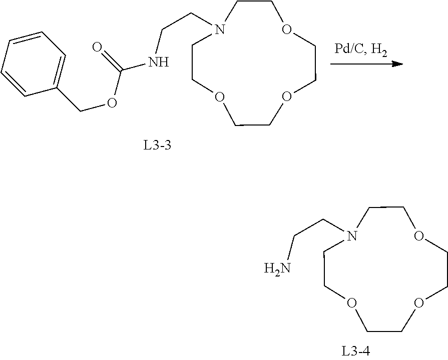 [SMILES: O=C(NCCN1CCOCCOCCOCC1)OCc1ccccc1 ; NCCN1CCOCCOCCOCC1]

### 2-(1,4,7-trioxa-10-azacyclododecan-10-yl)ethan-1-amine (L3-4)

L3-3 was dissolved in 10 mL of EtOH. 1 g of Pd/C was added and the solution was stirred for 12 h under H₂. The solution was filtered, and the solvent was removed in vacuo. ¹H-NMR (400 Hz, CDCl₃): δ 4.76 (2H, s), 3.44-3.22 (m, 12H), 2.67-2.52 (m, 6H). HRMS (ESI) calculated for C₁₀H₂₃N₂O₃[M+H]: 219.1704, found: 219.1706.

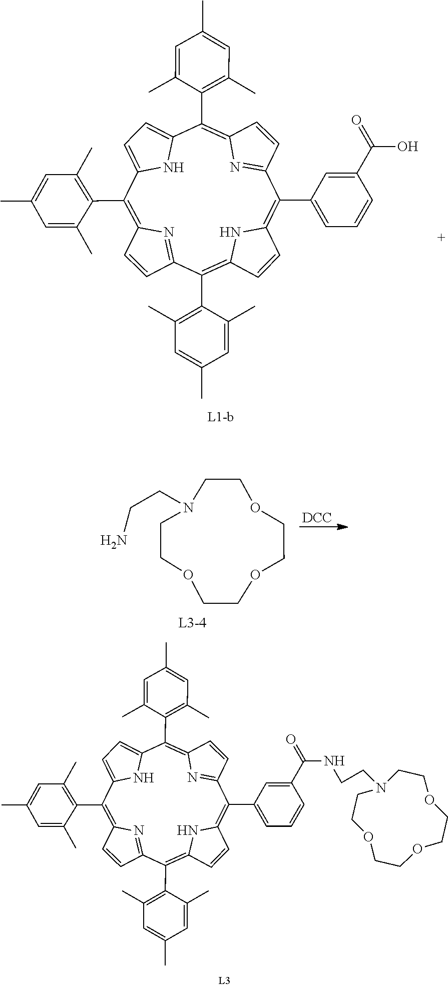

### 2-(1,4,7-trioxa-10-azacyclododecan-10-yl)ethyl3-(10,15,20-trimesitylporphyrin-5-yl) benzoate (L3)

L3-b (200 mg) was dissolved in 10 mL anhydrous CH₂Ch, then NHS (68 mg) and DCC (60 mg) were add. The solution was heated to 40° C. and stirred for 24 hours, followed up by adding 200 mg of L3-4 and another 24 hours stirring at 40° C. The solution was washed with saturated aqueous Na₂CO₃ solution (50 mL) and H₂O (50 mL×3), dried over anhydrous sodium sulfate. The solvent was removed in vacuo. The product was purified by prep-TLC (CH₂Cl₂/MeOH=10/1, R_{f}=0.3). ¹H-NMR (400 Hz, CDCl₃): δ 8.82-8.75 (dd, J=8.2 Hz, 4.7 Hz, 4H), 8.69 (s, 4H), 8.30-8.18 (dd, J=8.3 Hz, 4.6 Hz, 4H), 7.26 (s, 6H). 3.70 (t, J=3.9 Hz, 2H), 2.74-2.56 (m, 18H), 2.23 (s, 9H), 1.86 (s, 18H). ¹³C-NMR (101 MHz, CDCl₃): δ 167.9, 144.9, 139.4, 138.3, 138.1, 137.7, 134.3, 134.2, 127.8, 126.0, 117.7, 70.7, 69.1, 54.8, 53.8, 52.7, 50.5, 38.3, 33.9, 25.6, 25.0, 21.7, 21.4. HRMS (ESI) calculated for C₆₄H₆₉N₆O₄[M+H]: 985.5375, found: 985.5375.

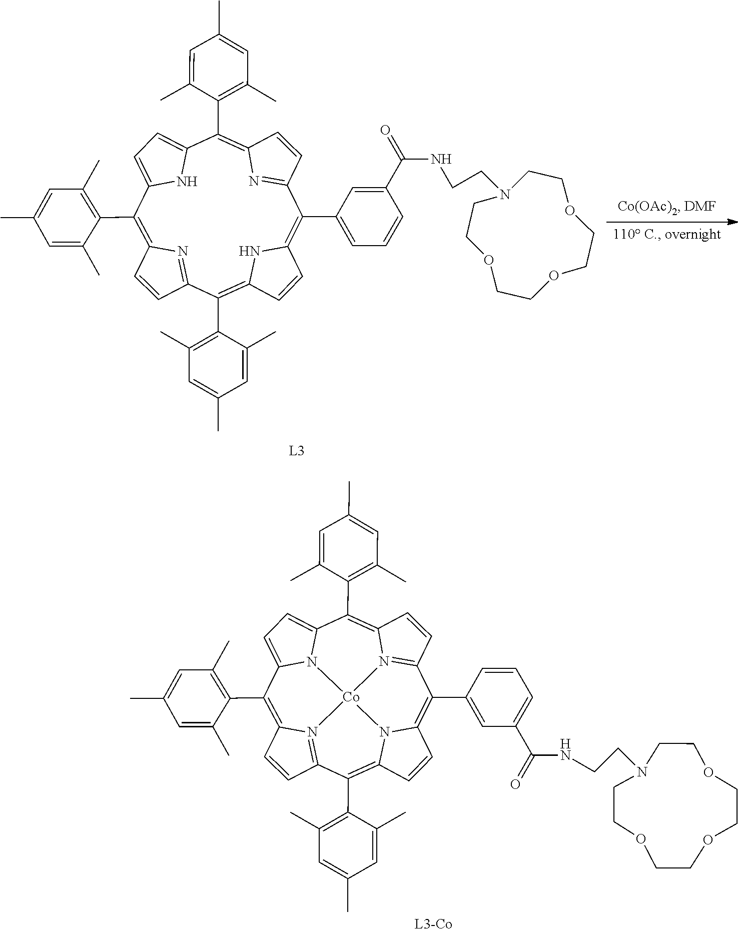

### Synthesis of L3-Co

L3 (100 mg) was dissolved in 10 mL of DMF. 80 mg of Co(OAc)₂ was added. The mixture was stirred at 150° C. for 12 h. After the reaction mixture was cooled to room temperature, 40 mL of CH₂Cl₂ was added. The solution was washed with saturated aqueous NaHCO₃ solution (50 mL) and H₂O (50 mL×3), dried over anhydrous Na₂SO₄. The solvent was removed in vacuo. NMR measurement was not accessible because cobalt/porphyrin is a paramagnetic compound. HRMS (ESI) calculated for CoC₆₄H₆₇N₆O₄[M+H⁺]: 1042.4551, found: 1042.4550.

### Synthesis of L4

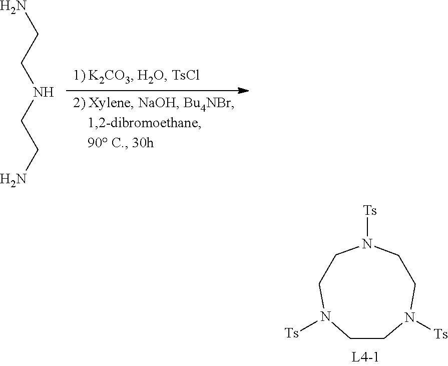 [SMILES: NCCNCCN ; Cc1ccc(S(=O)(=O)N2CCN(S(=O)(=O)c3ccc(C)cc3)CCN(S(=O)(=O)c3ccc(C)cc3)CC2)cc1]

### 1,4,7-tritosyl-1,4,7-triazonane (L4-1)

In a 1 L flask, diethylenetriamine (2.7 g, 26 mmol) and p-toluenesulfonyl chloride (16 g, 80 mmol) were added to a solution of K₂CO₃ (24.3 g, 250 mmol) dissolved in distilled water (300 mL) with vigorous stirring. After the mixture was stirred at 90° C. for 1 hour, xylene (240 mL), NaOH (19.15 g, 480 mmol), Bu₄NBr (0.5 g), and 1,2-dibromoethane (4 mL, 46 mmol) were added. The mixture was stirred at 90° C. for 30 h, during which, two portions of additional 1,2-dibromoethane (4 mL, 46 mmol) were respectively added at 4 h and 12 h after the first addition. The mixture was cooled to room temperature, and the formed precipitates were separated by filtration. Washing the white solids by water gave L4-1 in 79% yield (12.5 g). ¹H-NMR (400 MHz, CDCl₃) δ 7.70 (d, J=8.0 Hz, 6H), 7.32 (d, J=8.0 Hz, 6H), 3.42 (s, 12H). 2.43 (s, 9H). The spectral data for this compound match that reported in the literature.

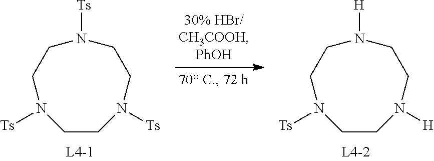 [SMILES: Cc1ccc(S(=O)(=O)N2CCN(S(=O)(=O)c3ccc(C)cc3)CCN(S(=O)(=O)c3ccc(C)cc3)CC2)cc1 ; Cc1ccc(S(=O)(=O)N2CCNCCNCC2)cc1]

### 1-tosyl-1,4,7-triazonane (L4-2)

A 500 mL Schlenk flask was charged with L4-1 (7.8 g, 21.2 mmol), phenol (15 g) and a 30% solution of HBr in AcOH (200 mL). The suspension was slowly warmed to 90° C. under stirring over 1 h. At 70° C., a strong gas evolution took place, and all solids were completely dissolved. Gas evolution ceased after heating at 90° C. for 2-3 hours and a white precipitate of the product the adduct of L4-1 and HBr appeared. The reaction mixture was stirred at 90° C. for 3 days, then cooled to room temperature and left to stand overnight. The precipitate was filtered and collected, washed with diethyl ether (3×20 mL), and dissolved in 1.5 M aqueous NaOH solution to give a rose solution, which was extracted with CHCl₃ (6×15 mL). Combined extracts were dried over MgSO₄, concentrated to give 4.55 g (76% yield) of L4-2 as a yellow oil. ¹H-NMR (400 MHz, CDCl₃) δ 7.68 (d, J=8.3 Hz, 2H), 7.30 (d, J=8.0 Hz, 2H), 3.23-3.14 (m, 4H), 3.11-3.04 (m, 4H), 2.89 (s, 4H), 2.42 (s, 3H), 1.81 (s, 3H). The spectral data for this compound match that reported in the literature.

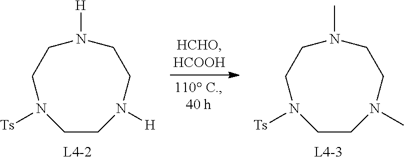 [SMILES: Cc1ccc(S(=O)(=O)N2CCNCCNCC2)cc1 ; Cc1ccc(S(=O)(=O)N2CCN(C)CCN(C)CC2)cc1]

### 1,4-dimethyl-7-tosyl-1,4,7-triazonane (L4-3)

Compound L4-2 (4.55 g. 16 mmol) was mixed with water (4 mL). The resulting suspension was cooled to 0° C. and 37% formaldehyde (HCHO) (15 mL) was added dropwise under stirring until all solid was dissolved (˜20 min). Then 97% formic acid (HCOOH) (15 mL) was added dropwise over a 10 min period and the reaction mixture was stirred for another 30 min at 0° C. The reaction was allowed to reach room temperature and refluxed for 15 h. After cooling to room temperature, 33% HCl (10 mL) was added to the reaction mixture and the volatiles were removed in vacuo. The residue was dissolved in 1.5 M aqueous NaOH solution (20 mL) (pH >10) and the resulting suspension was extracted with CHCl₃ (6×15 mL). The combined extracts were concentrated and dried to give 4.6 g (94% yield) of L4-3 as a pale yellow solid. ¹H-NMR (400 MHz, CDCl₃) δ 7.67 (d, J=8.3 Hz, 2H), 7.30 (d, J=8.0 Hz, 2H), 3.34-3.16 (m, 4H), 3.01-2.89 (m, 4H), 2.76 (s, 4H), 2.43 (s, 6H), 2.42 (s, 3H). The spectral data for this compound match that reported in the literature.

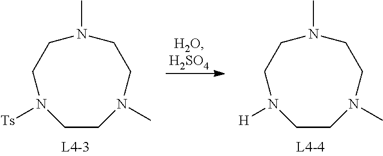 [SMILES: Cc1ccc(S(=O)(=O)N2CCN(C)CCN(C)CC2)cc1 ; CN1CCNCCN(C)CC1]

### 1,4-dimethyl-1,4,7-triazonane (L4-4)

Compound L4-3 (4.6 g, 15.0 mmol) was added to 98% H₂SO₄ (20 mL) in a 50 mL one-necked flask. The mixture was stirred at 120° C. for 36 h. The resulting dark brown solution was cooled to room temperature and poured into a flask containing crushed ice. An aqueous solution of NaOH (12 M) was added slowly with ice cooling until the pH >12. The resulting dark brown solution was extracted with CHCl₃ (6×30 mL). The combined organic layers were dried over MgSO₄ and concentrated to give crude product. The crude product was further purified by vacuum distillation using (0.1-0.2 mmHg, 50-60° C.) to give 1.5 g (67% yield) of pure ligand L4-4 as a colorless oil. ¹H-NMR (400 MHz, CDCl₃) δ 2.99 (t, J=6.0 Hz, 4H), 2.69 (t, J=6.0 Hz, 4H), 2.52 (s, 4H). 2.45 (s, 6H). The spectral data for this compound match that reported in the literature.

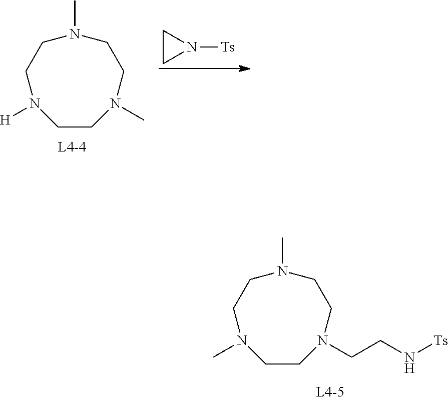

### N-(2-(4,7-dimethyl-1,4,7-triazonan-1-yl)ethyl)-4-methylbenzenesulfonamide (L4-5)

(L4-4) (500 mg) was dissolved in 50 ml toluene. 1-Tosylaziridine (460 mg) in 7 mL of toluene was added dropwise into the solution within 2 h under N₂ atmosphere. The mixture was stirred for another 12 h. The reaction mixture was washed with water (4×50 mL) to remove unreacted 1-tosylaziridine. Organic solvents were removed under reduced pressure to give 930 mg (97% yield) of L4-5. Spectral data (L4-5): ¹H-NMR (400 MHz, CDCl₃) δ: 7.63 (d, 2H, J=4.1 Hz), 7.17 (d, 2H, J=4.1 Hz), 2.86 (t, 2H, J=6.2 Hz), 2.60 (t, 2H, J=6.2 Hz), 2.52-2.46 (m, 12H), 2.29-2.22 (m, 14H). ¹³C-NMR (101 MHz, CDCl₃) δ:142.4, 137.7, 129.4, 128.9, 58.3, 57.3, 55.6, 55.0, 53.5, 46.3, 42.7, 21.4. HRMS (ESI) calculated for C₁₇H₃₁N₄SO₂ [M+H⁺]: 355.2163, found: 355.2163.

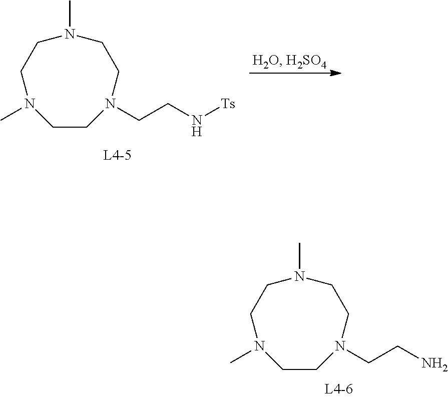 [SMILES: Cc1ccc(S(=O)(=O)NCCN2CCN(C)CCN(C)CC2)cc1 ; CN1CCN(C)CCN(CCN)CC1]

### 2-(4,7-dimethyl-1,4,7-triazonan-1-yl)ethan-1-amine (L4-6)

(L4-5) (930 mg) and 18 mol/L sulfuric acid (5 mL) were mixed and stirred at 100° C. for 3 days. After cooling down to 0° C., NaOH was added to the reaction mixture until pH >12. The aqueous phase was extracted with CHCl₃ (4×100 mL). The combined CHCl₃ layers were dried over MgSO₄. The solvent was removed under reduced pressure, giving 280 mg of L4-6 (58% yield) as a colorless viscous oil. Spectral data (L4-6): ¹H-NMR (401 MHz, CDCl₃): δ 2.62-2.50 (m, 16H), 2.35-2.20 (m, 6H)¹³C-NMR (101 MHz, CDCl₃): δ 61.8, 57.3, 56.9, 56.3, 46.5, 40.3. HRMS (ESI) calculated for C₁₀H₂₅N₄[M+H⁺]: 201.2074. found: 201.2073.

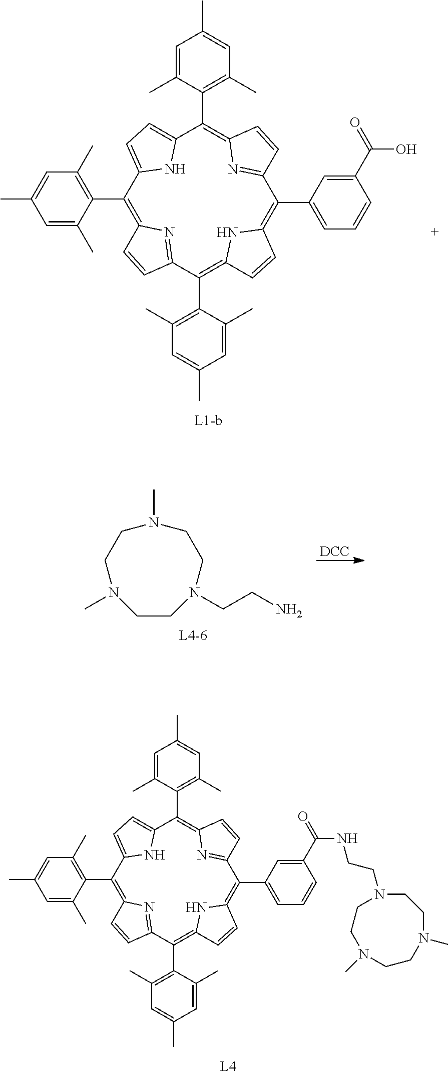

### N-(2-(4,7-dimethyl-1,4,7-triazonan-1-yl)ethyl)-3-(10,15,20-trimesitylporphyrin-5-yl)benzamide (L4)

L2-b (200 mg) was dissolved in 10 mL anhydrous CH₂Cl₂, then NHS (68 mg) and DCC (60 mg) were add. The solution was heated to 40° C. and stirred for 24 h, followed by adding 200 mg of L4-6 and stirring for another 24 h at 40° C. The solution was washed with saturated aqueous Na₂CO₃ solution (50 mL) and H₂O (50 mL×3), dried over anhydrous Na₂SO₄. The solvent was removed in vacuo. The product was purified by prep-TLC (CH₂Cl₂/MeOH/TEA=10/1/0.1, R_{f}=0.2). ¹H-NMR (400 Hz, CDCl₃): δ 8.66-8.60 (m, 8H), 8.19-8.21 (m, 3H), 7.75-7.76 (m, 1H), 7.27 (s, 6H), 3.58 (t, J=6.9 Hz, 2H), 2.82-2.79 (t, J=6.9 Hz, 2H), 2.55-1.98 (m, 25H), 1.80-1.75 (m, 18H). ¹³C-NMR (101 MHz, CDCl₃): δ 168.2, 142.1, 139.4, 139.3, 138.0, 128.5, 127.7, 126.8, 127.7, 118.5, 117.9, 45.5, 33.93, 29.7, 29.6, 21.8, 21.7, 21.6, 21.5, 21.4. HRMS (ESI) calculated for C₆₄H₇₁N₈O[M+H]: 967.5746, found: 967.5743.

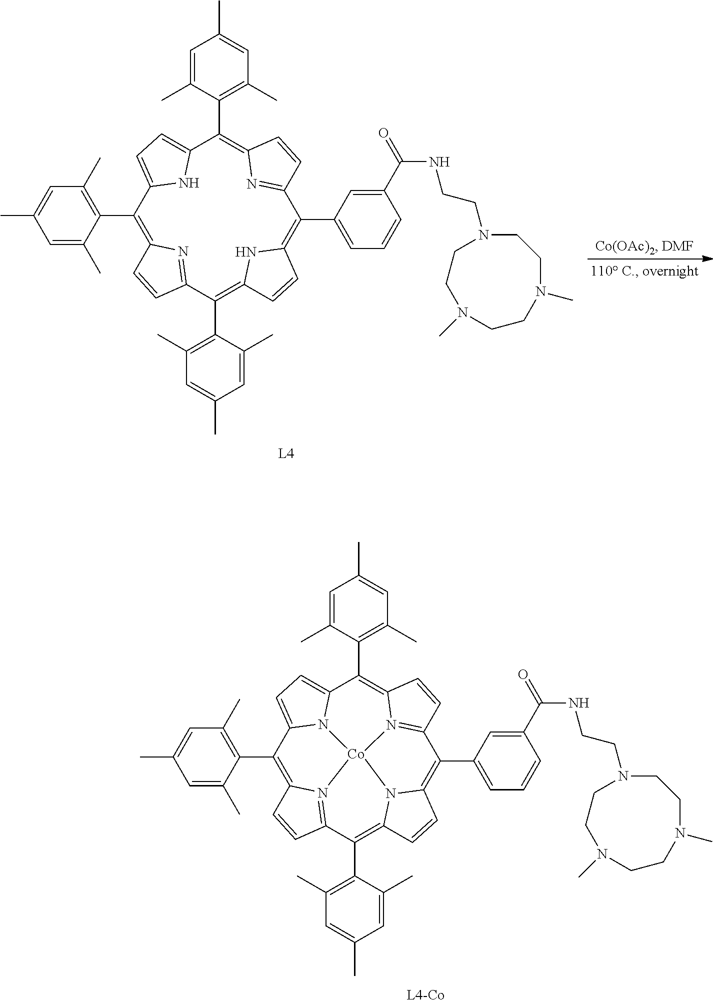

### Synthesis of L4-Co

L3 (100 mg) was dissolved in 10 mL of DMF and 80 mg of Co(OAc)₂ was added. The mixture was stirred at 150° C. for 12 h. After the reaction mixture was cooled to room temperature, 40 mL of CH₂Cl₂ was added. The solution was washed with saturated aqueous solution of NaHCO₃ (50 mL) and H₂O (50 mL×3), dried over anhydrous Na₂SO₄. The solvent was removed in vacuo. NMR measurement was not accessible because cobalt/porphyrin is a paramagnetic compound. HRMS (ESI) calculated for CoC₆₄H₆₉N₈O[M+H⁺]: 1024.4921, found: 1024.4920.

### Synthesis of (3-acrylamidopropyl)trimethylammonium trifluoromethanesulfonate (2j)

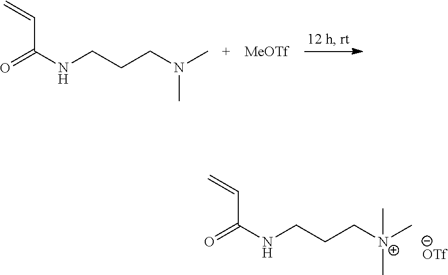

N-(3-(dimethylamino)propyl)acrylamide (2h) (5 g, 32 mmol) was dissolved in 50 mL CH₂Cl₂. Methyl trifluoromethanesulfonate (MeOTf) (5.2 g, 32 mmol) in 5 mL CH₂Cl₂ was added dropwise into the solution within 2 h under N₂ atmosphere. The mixture was stirred for another 12 h. The reaction mixture was washed with hexane (4×50 mL) to remove unreacted 2 h and MeOTf. Organic solvents were removed under reduced pressure to give 8.5 g (83% yield) of 2j. Spectral data (2j): ¹H-NMR (400 MHz, DMSO-d₆) δ: 6.27-6.03 (m, 2H), 5.68 (dd, J=9.6, 2.0 Hz, 1H), 3.36-3.17 (m, 4H), 3.02 (s, 9H), 2.04-1.86 (m, 2H). ¹³C-NMR (101 MHz, CDCl₃) δ: ¹³C NMR (101 MHz, DMSO-d₆) δ 168.6, 129.8, 127.5, 119.7 (q, J=312 Hz), 64.3, 64.2, 64.2, 54.0, 53.0, 52.9, 52.9, 36.1, 36.0, 36.0, 22.6.

### Example 2: LACoP-Initiated Stereocontrolled Living Radical Polymerization (LRP)

### General Procedure for Co Por-Initiated Stereocontrolled Living Radical Polymerization (LRP)

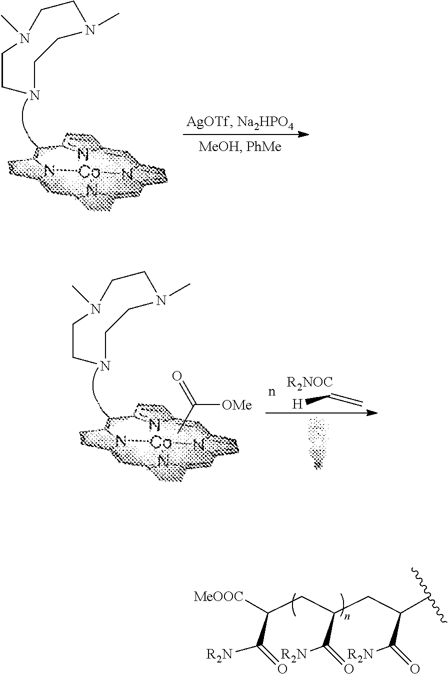

A toluene solution (10.0 mL) of Co^{II}/por (0.01 mmol), AgOTf (0.04 mmol, 10.3 mg), Na₂HPO₄ (0.08 mmol, 11.36 mg), and MeOH (1.0 mL) was degassed through three freeze-pump-thaw cycles and refilled with CO (1 atm). The mixture was then stirred for 7 h in the dark at room temperature. The inorganic impurities were removed by water extraction. The remaining organic solvent was evaporated and followed by addition of acrylamide (4 mmol), specified amount of Lewis acids (LAs), and solvent (4 mL). After three freeze-pump-thaw cycles, the flask was refilled with N₂ and irradiated with visible light for 5 hours. The polymer product was purified through dialysis in MeOH.

The monomer conversion and tacticity of polymer were determined by ¹H-NMR spectra in DMSO-d₆. The number-average molecular weight (Mₙ) and dispersity index (D) were determined using GPC analysis in DMF (0.01 mol/L LiBr) eluent. Exemplary tacticity data under various conditions (e.g., alternative solvents, temperatures, light intensity. Lewis acids, initiation conditions, varied monomers, are provided herein (Tables 1-8).

TABLE 1
Tacticity data obtained from polymerizations in different solvents

| solvent (volume | meso diads | monomer | Mₙ | |
|---|---|---|---|---|
| ratio) | (%) | conversion (%) | (kDa) | Ð |
| benzene | 60 | 47 | 82.5 | 1.31 |
| MeOH | 85 | 78 | 75.9 | 1.29 |
| DMSO | 50 | 42 | 85.6 | 1.46 |
| DMF | 51 | 49 | 92.3 | 1.57 |
| HFIP | — | 0 | — | — |
| PhCF₃ | 76 | 11 | 72.9 | 1.49 |
| benzene:MeOH (1:9) | 85 | 79 | | 1.22 |
| benzene:MeOH (1:1) | 76 | 76 | 82.5 | 1.40 |

Other conditions: [DMAA]₀:[1b]₀:[La(OTf)₃]₀ = 400:1:10 in 4 mL of solvent at room temperature with a light intensity of 3 mW/cm².
HFIP: hexafluoro-2-propanol.
PhCF₃: trifluorotoluene

TABLE 2
Tacticity data obtained from polymerizations
at different temperatures

La(OTf)₃ (mol %)

| | | | | |
|---|---|---|---|---|
| 0.5 | 1 | 2.5 | 5 | 10 |

| | |
|---|---|
| temperature | meso diads (%) |

| | | | | | |
|---|---|---|---|---|---|
| room temperature | 79 | 83 | 90 | 93 | 95 |
| 4° C. (cold room) | 78 | 83 | 86 | 93 | 95 |

Other conditions: [DMAA]₀:[1b]₀ = 400:1 in 4 mL of MeOH; light intensity: 3 mW/cm²; La(OTf)₃ mole fraction was relative to monomer.

TABLE 3
Tacticity data obtained based on light intensity

| light intensity | meso diads | monomer conversion | Mₙ | |
|---|---|---|---|---|
| (mW/cm⁻²) | (%) | (%) | (kDa) | Ð |
| 0.1 | — | 0 | — | — |
| 0.3 | 83 | 51 | 58.9 | 1.46 |
| 1.0 | 83 | 79 | 71.6 | 1.29 |
| 3.0 | 83 | 89 | 83.2 | 1.21 |
| 5.0 | 83 | 85 | 79.5 | 1.43 |

Other conditions: [DMAA]₀:[1e]₀:[La(OTf)₃]₀ = 400:1:4 in 4 mL of MeOH at room temperature.

TABLE 4
Tacticity data obtained from polymerizations uzing different Las

| | meso diads | monomer conversion | Mₙ | |
|---|---|---|---|---|
| LA | (%) | (%) | (kDa) | Ð |
| La(OTf)₃ | 83 | 83 | 82.5 | 1.31 |
| Y(OTf)₃ | 82 | 78 | 75.9 | 1.28 |
| Sc(OTf)₃ | 80 | 79 | 85.6 | 1.44 |
| Yb(OTf)₃ | 81 | 84 | 92.3 | 1.32 |
| La(NTf₂)₃ | 84 | 89 | 91.1 | 1.51 |
| Yb(NTf₂)₃ | 82 | 86 | 84.3 | 1.47 |
| LiNTf₂ | 51 | 62 | 59.6 | 1.26 |
| LiOTf | 51 | 65 | 55.5 | 1.29 |
| AlCl₃ | — | 0 | — | — |

Other conditions: [DMAA]₀:[1e]₀:[LA]₀ = 400:1:4 in 4 mL of MeOH at room temperature.

TABLE 5
Tacticity data obtained from polymerizations
under different initiation conditions

La(OTf)₃ (mol %)

| | | | | |
|---|---|---|---|---|
| 0.5 | 1 | 2.5 | 5 | 10 |

| | |
|---|---|
| initiation method | meso diads (%) |

| | | | | | |
|---|---|---|---|---|---|
| Co^{II} (L1-Co) + V-70, | 60 | 71 | 85 | 86 | 89 |
| thermal (50° C.) | | | | | |
| Co^{II} (L1-Co) + TPO, photo | 57 | 65 | 72 | 79 | 87 |
| Co^{III} (1b), photo | 77 | 82 | 88 | 91 | 93 |

Other conditions: [DMAA]₀:[Co]₀:[initiator] = 400:1:1 in 4 mL of MeOH. Light intensity: 3 W/cm². La(OTf)₃ mole fraction is with respect to monomer. V-70: 2,2′-azobis(4-methoxy-2,4-dimethylvaleronitrile); TPO: diphenyl(2,4,6-trimethylbenzoyl)phosphine oxide.

TABLE 6
Tacticity data obtained from polymerizations
with different initiators

| | La(OTf)₃ (mol %) | | | | |
|---|---|---|---|---|---|
| | 0.5 | 1 | 2.5 | 5 | 10 |
| Initiator | meso diads (%) | | | | |
| TPO | 57 | 60 | 67 | 73 | 87 |
| 1a | 60 | 69 | 77 | 85 | 88 |
| 1a + TMTZC | 60 | 69 | 77 | 85 | 88 |
| (1:1) | | | | | |
| 1b | 75 | 79 | 85 | 90 | 92 |
| 1c | 77 | 82 | 88 | 91 | 93 |
| 1d | 60 | 64 | 74 | 83 | 87 |
| 1e | 79 | 83 | 90 | 93 | 95 |

Other conditions: [DMAA]₀:[initiator]₀ = 400:1 in 4 mL of MeOH at room temperature; light intensity: 3 mW/cm²; La(OTf)₃ mole fraction was relative to monomer. TMTZC: 1,4,7-trimethyl-1,4,7-triazacyclononane.

TABLE 7
Tacticity data obtained from 1a or 1e-initiated
polymerizations of different monomers

La(OTf)₃ (mol %)

| | | | | |
|---|---|---|---|---|
| 0.5 | 1 | 2.5 | 5 | 10 |

initiator

| | | | | |
|---|---|---|---|---|
| 1e/1a | 1e/1a | 1e/1a | 1e/1a | 1e/1a |

| | |
|---|---|
| monomer | meso diads (%) |

| | | | | | |
|---|---|---|---|---|---|
| DMAA | 51/51 | 84/69 | 90/77 | 93/85 | 95/88 |
| DEAA | 54/54 | 83/71 | 83/73 | 91/82 | 94/86 |
| NIPAM | 48/48 | 80/69 | 86/73 | 90/82 | 92/89 |
| HEAA | 43/43 | 67/49 | 71/51 | 74/59 | 80/65 |

Other conditions: [monomer]₀:[initiator]₀ = 400:1 in 4 mL of MeOH at room temperature; light intensity: 3 mW/cm²; La(OTf)₃ mole fraction was with relative to monomer.

TABLE 8
Tacticity, molecular weight and polydispersity
data obtained from different monomer

| Monomer | meso diads (%) | Mₙ (kDa) | Ð |
|---|---|---|---|
| 2a | 93 | 72.9 | 1.40 |
| 2b | 91 | 48.9 | 1.31 |
| 2c | 90 | 45.2 | 1.28 |
| 2d | 82 | 22.7 | 1.34 |
| 2e | 83 | 45.5 | 1.56 |
| 2f | 82 | 116.2 | 1.32 |
| 2g | 85 | 27.8 | 1.51 |
| 2h | 81 | 30.4 | 1.45 |
| 2i | 74 | 14.4 | 1.31 |
| 2j* | 79 | 44.4 | 1.45 |
| 2k | 62 | 11.5 | 1.41 |

Other conditions: [DMAA]₀:[1e]₀:[LA]₀ = 400:1:20 in 4 mL of MeOH at room temperature.
*GPC tested in 10 mM LiOTf in DMF solution as eluent.

### Example 3: Evidence of Free Radical Polymerization Mechanism

### Quenching of polymerization by (22,6,6-tetramethylpiperidin-1-yl)oxyl (TEMPO)

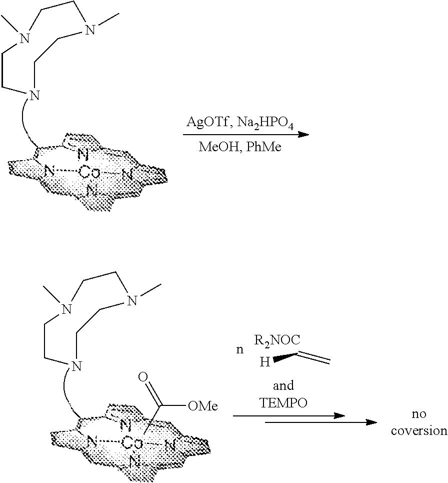

Reaction was performed in accordance with the procedure described in Example 2 herein, wherein the procedure further comprised the addition of TEMPO (4.0 mmol). The monomer conversion was determined by ¹H-NMR spectra in DMSO-d₆ using anisole as an internal standard. No polymerization was detected according to the conversion based on NMR analysis.

Light-Regulated Switching (on/Off) of Polymerization

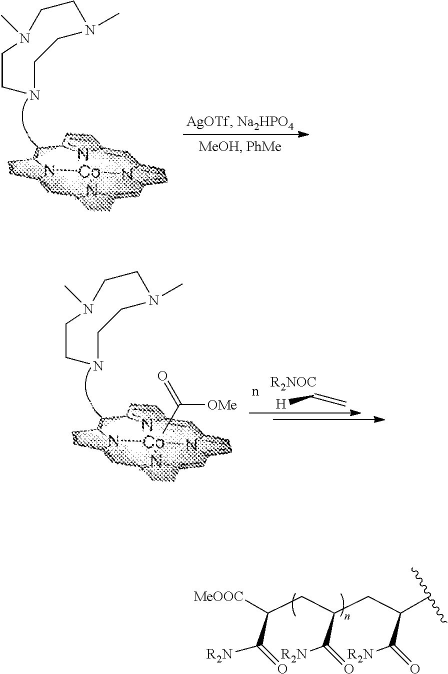

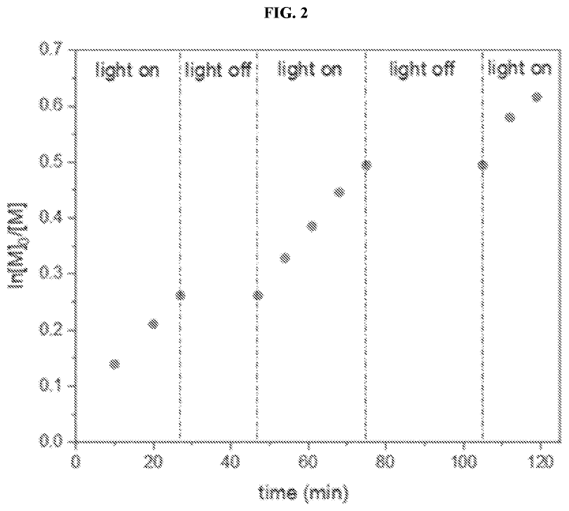

Reaction was performed in accordance with the procedure described in Example 2 herein, wherein the procedure comprised periodic visible light irradiation. Aliquots were taken using degassed syringes under N₂ flow for ¹H-NMR and GPC tests. The monomer conversion was determined by ¹H-NMR spectra in DMSO-d₆ using anisole as an internal standard. Relationship between ln[M]₀/[M] over time was shown in FIG. 2.

### Example 4: Triad/Diad Tacticity Analysis

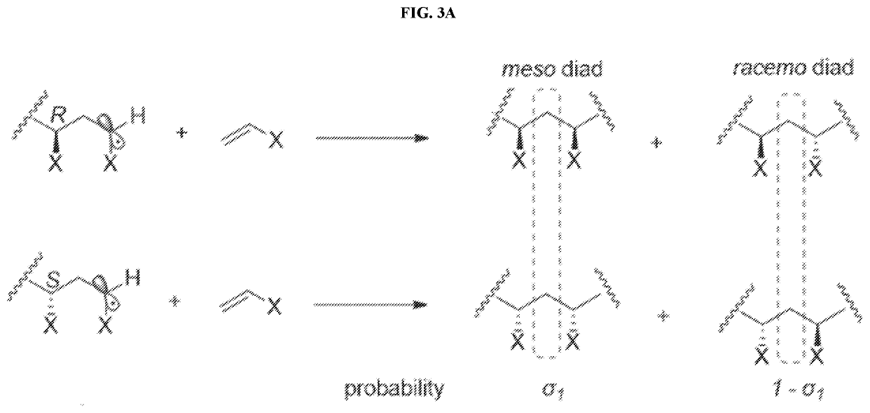

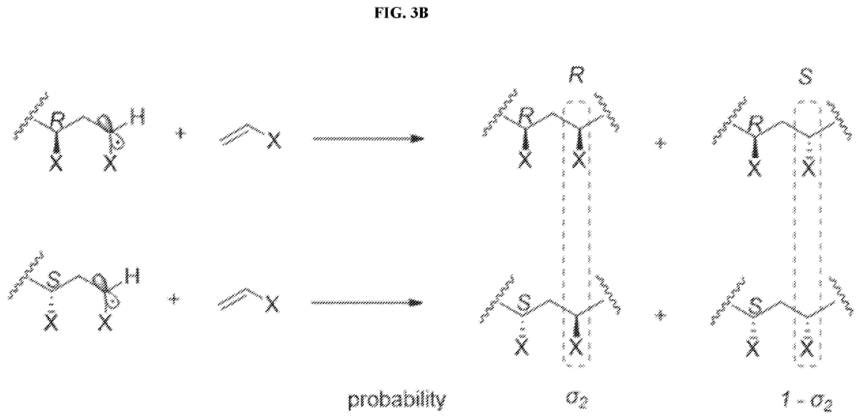

Theoretically, there are two possible pathways for the tacticity control process: chain-end control and catalyst control. In chain-end control process, an incoming monomer's addition depends on the stereochemistry of the latest enchained unit. As a result, the possibility to form different kinds of diads (m or r) is consistent (FIG. 3A). In catalyst control process, stereochemistry of radical addition is independent of the latest enchained unit and dependent on the catalyst (FIG. 3B), leading to the consistent possibility for the formation of certain configuration (R or S). As a result, chain-end control and catalyst control process can provide polymers with different diad and triad distribution.

For chain end control, assuming that the probabilities of forming a meso and raceme diad are σ₁ and (1−σ₁), respectively, the following equations are obtained:

$$( m ) = {σ}_{1} \qquad ( Eq . 1 )$$

$$( mm ) = {σ}_{1}^{2} \qquad ( Eq . 2 )$$

Combining Eqs. 1-2:

$$\frac{( mm )}{{( m )}^{2}} = 1 \qquad ( Eq . 3 )$$

For catalyst control, assuming that the probability of forming an R configuration is σ₂ and the probability of forming an S configuration is (1-σ₂), the following equations are obtained:

$$( m ) = ( RR ) + ( SS ) = {σ}_{2}^{2} + {( 1 - {σ}_{2} )}^{2} \qquad ( Eq . 4 )$$

$$( mm ) = ( RRR ) + ( SSS ) = {σ}_{2}^{3} + {( 1 - {σ}_{2} )}^{3} \qquad ( Eq . 5 )$$

Simplifying Eqs. 4-5:

$$( m ) = {( {σ}_{2} + 1 - {σ}_{2} )}^{2} - 2 {σ}_{2} ( 1 - {σ}_{2} ) = 1 - 2 {σ}_{2} ( 1 - {σ}_{2} ) \qquad ( Eq . 6 )$$

$$( mm ) = {( {σ}_{2} + 1 - {σ}_{2} )}^{3} - 3 {σ}_{2} ( 1 - {σ}_{2} ) = 1 - 3 {σ}_{2} ( 1 - {σ}_{2} ) \qquad ( Eq . 7 )$$

Combining Eqs. 6-7:

$$\frac{1 - ( mm )}{1 - ( m )} \cdot \frac{2}{3} = 1 \qquad ( Eq . 8 )$$

The data of triad and diad analysis are provided below according to Eq. 3 and Eq. 8, and show that LACoP-initiated stereocontrolled LRPs followed a chain-end control mechanism.

TABLE 9
Diad and triad analysis of polyDMAA

| catalytic system | m | mm | $\frac{( mm )}{{( m )}^{2}}$ | $\frac{1 - ( mm )}{1 - ( m )} \cdot \frac{2}{3}$ |
|---|---|---|---|---|
| 1e + 1.0 mol % La(OTf)₃ | 83 | 67 | 0.97 | 1.33 |
| 1e + 2.5 mol % La(OTf)₃ | 90 | 81 | 1.00 | 1.27 |
| 1e + 5.0 mol % La(OTf)₃ | 93 | 84 | 0.97 | 1.62 |
| 1e + 10.0 mol % La(OTf)₃ | 95 | 88 | 0.98 | 1.60 |
| 1c + 5.0 mol % La(OTf)₃ | 89 | 77 | 0.97 | 1.39 |
| 1c + 1.0 mol % La(OTf)₃ | 82 | 66 | 0.98 | 1.26 |

According to Table 9, LACoP-initiated stereocontrolled LRPs followed a chain-end control mechanism.

### Example 5: NMR Studies of the Interaction Between LA, Aza-Crown Ether Ligand, and Monomer

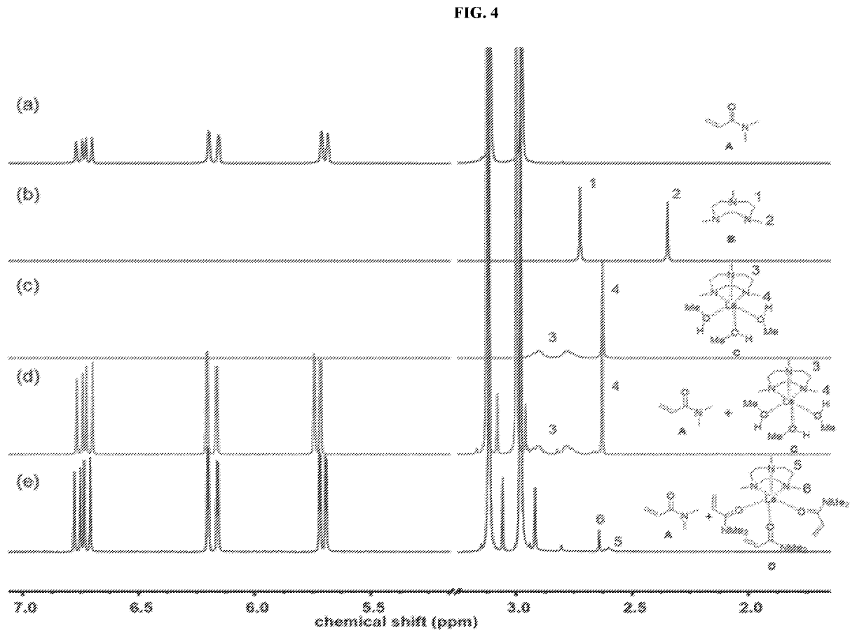

TMTZC was prepared, according to the literature, as a model compound to understand the interaction between 1e and rare-earth metal. As shown in FIG. 4B-4C, when TMTZC was mixed with 1.0 equiv. La(OTf)₃, peak 1 originating from TMTZC split into 2 sets of peaks (peaks 3), demonstrating the κ³ coordination of TMTZC to the central metal. The stability of La³⁺/TMTZC complex was further confirmed by adding 10.0 equiv. DMAA (FIG. 4D). When DMAA was added with 400.0 equiv. (FIG. 4E), at which the concentration was consistent with the polymerization conditions, the κ³ coordination was maintained evidenced by the presence of peaks 5 and 6 in despite of a changed coordination environment of La³⁺.

### Example 6: Kinetic Studies of LACoP-Initiated Polymerizations

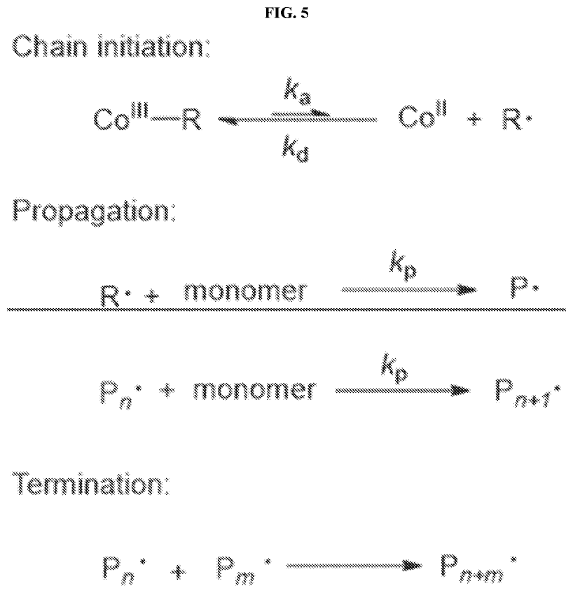

A mechanism of the radical polymerization disclosed herein has been proposed (FIG. 5). According to the pseudo-stead-state hypothesis (PSSH): the total concentration of active species [R·]ₜₒₜₐₗ, where

$${⌈ R \cdot ⌉}_{total} = {\sum }_{i = 0}^{∞} [ {P}_{n} \cdot ] ,$$

was assumed to be a constant in LRP.

$$Rate law of monomer : - \frac{d [ M ]}{dt} = {k}_{p} [ M ] {\sum }_{i = 0}^{∞} [ {P}_{n} \cdot ] = {{k}_{p} [ M ] [ R \cdot ]}_{total} \qquad ( Eq . 9 )$$

$$Solution of Eq . 9 : ln \frac{{[ M ]}_{0}}{[ M ]} = {{k}_{p} [ R \cdot ]}_{total} t \qquad ( Eq . 10 )$$

The product kₚ[R·]ₜₒₜₐₗ, namely apparent rate constant

$${k}_{p}^{app} ,$$

can be calculated from the slope of the pseudo-first-order kinetic plot of ln[M]₀/[M] versus t.

$${k}_{p}^{app}$$

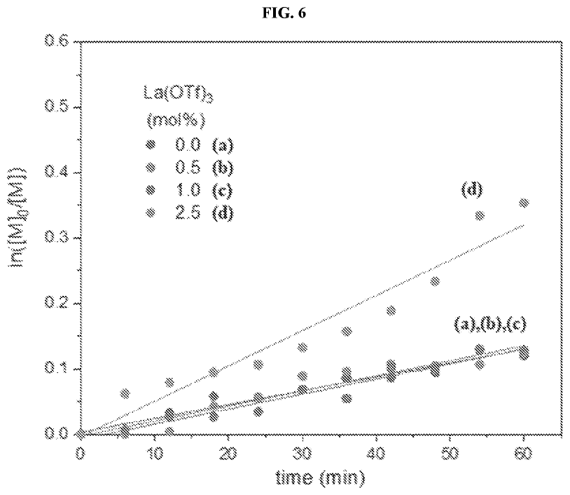

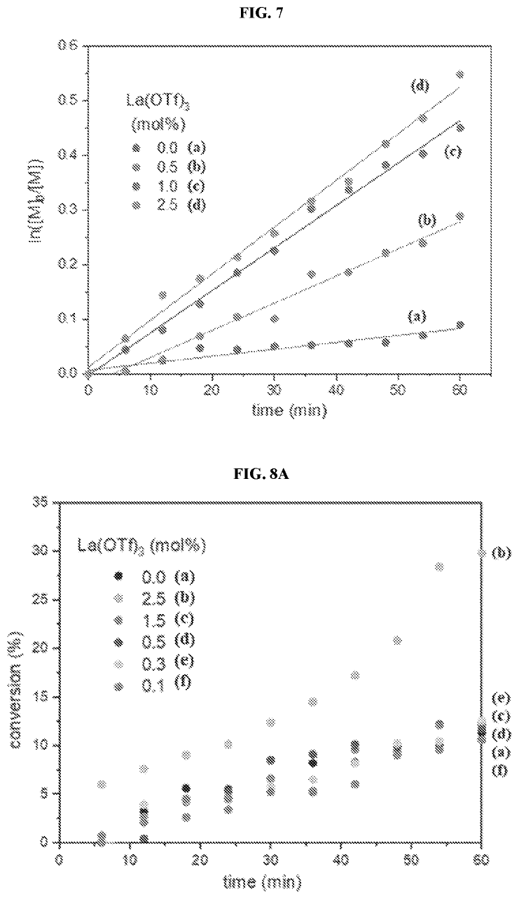

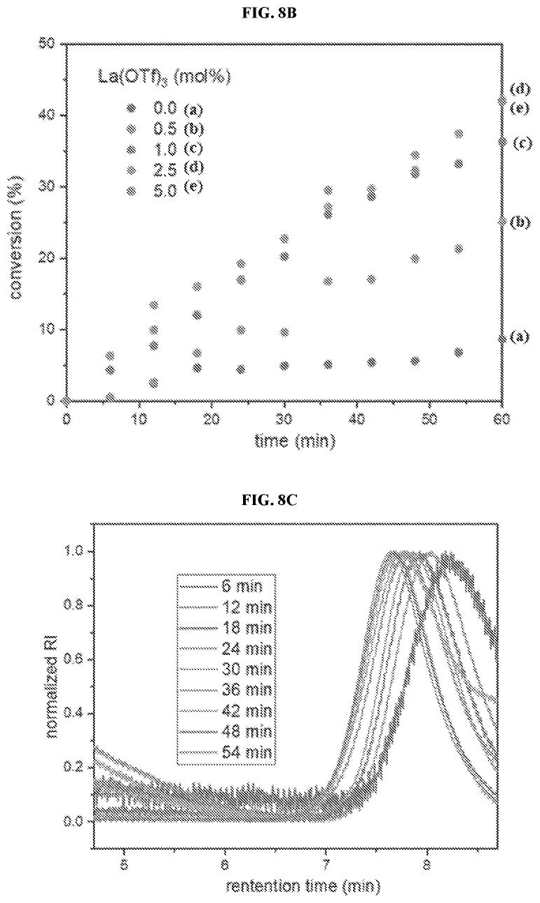

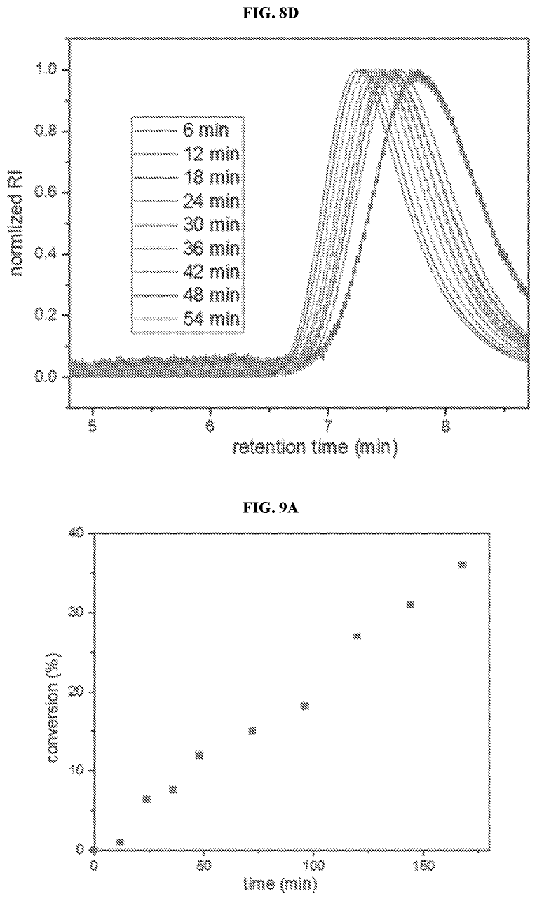

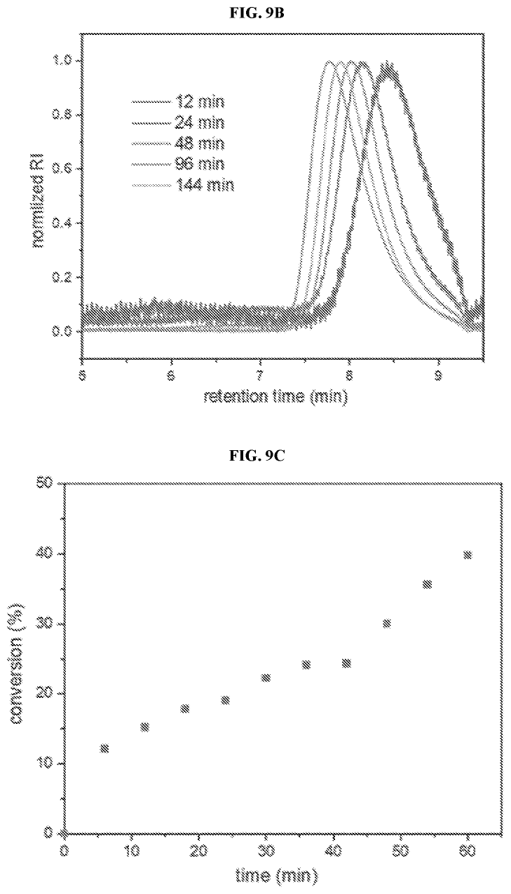

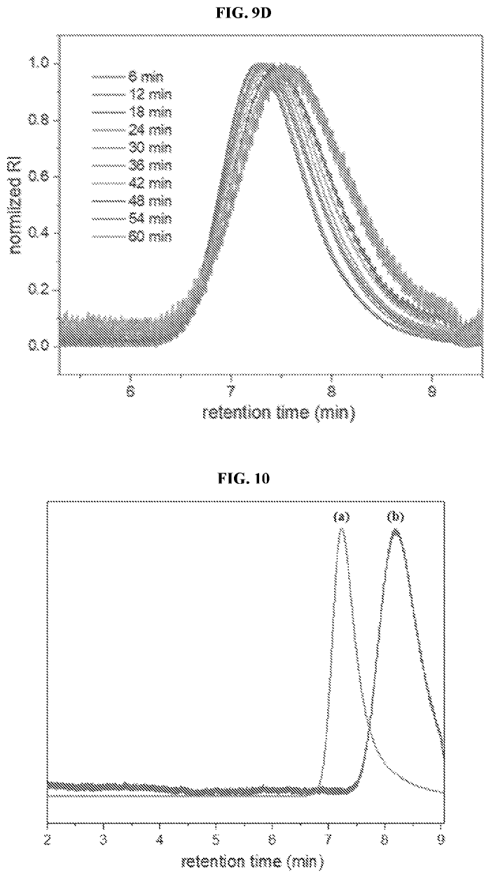

values of polymerizations initiated by 1a and 1e were calculated based on the slopes of ln[M]₀/[M] versus time plots (FIGS. 6-7). Exemplary plots depicting monomer as a function of time for various monomer polymerizations are also provided (FIGS. 8A-8D and FIGS. 9A-9D).

TABLE 10
$Apparent rate constant ( {k}_{p}^{app} ) of polymerizations at$ $various {La ( OTf )}_{3} loadings$

| La(OTf)₃ (mol %) | 0.0 | 0.5 | 1.0 | 2.5 |
|---|---|---|---|---|
| initiator | ${k}_{p}^{app} ( {10}^{- 3} {min}^{- 1} )$ | | | |
| 1a | 2.11 | 2.23 | 2.30 | 5.39 |
| 1e | 1.27 | 4.97 | 7.78 | 8.56 |

### Example 7: Diblock Copolymer Synthesis

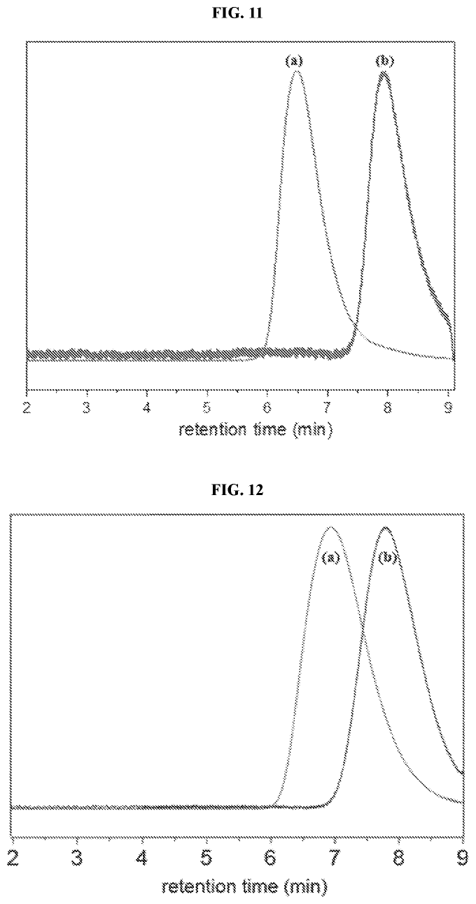

Reaction was performed in accordance with the procedure described in Example 2 herein. The irradiation time was subjected to 30 min. Aliquots were taken using degassed syringes under N₂ flow for ¹H-NMR and GPC tests. Monomer and solvent were removed under vacuum in dark. Different monomers (4 mmol) and MeOH (4 mL) were added under N₂ and irradiated under visible light for another 5 h (FIGS. 10-12).

### Example 8: Evaluation of Lewis Acid Co-Porphyrin (LACoPs) Mediated Living Radical Polymerization (LRP)

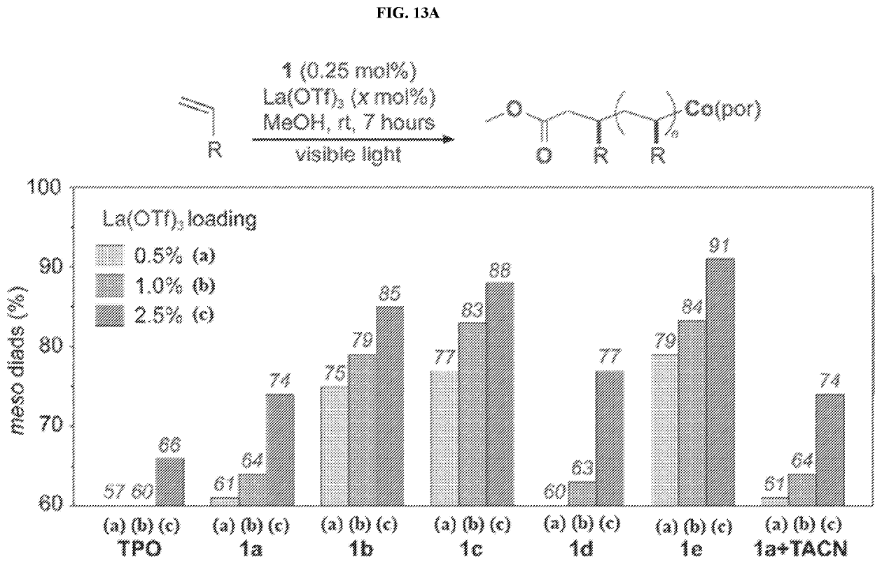

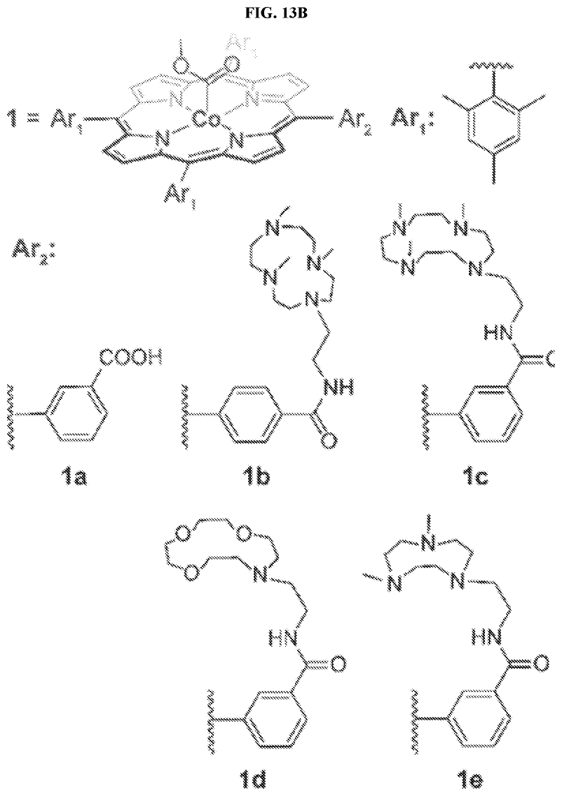

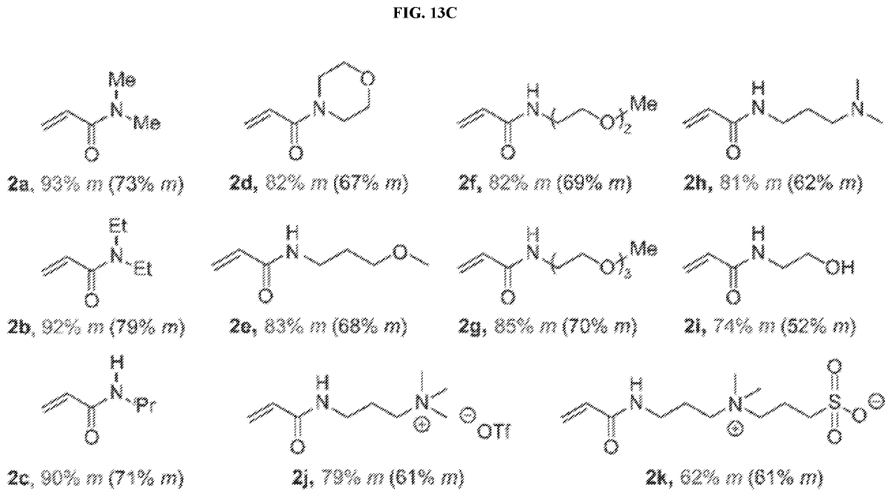

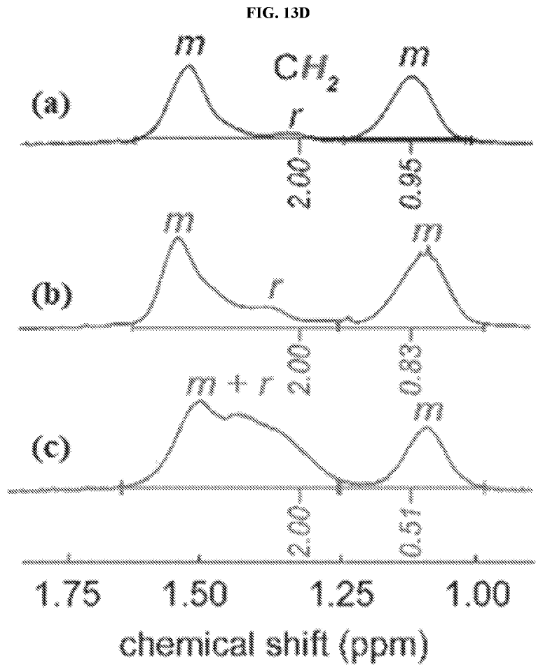

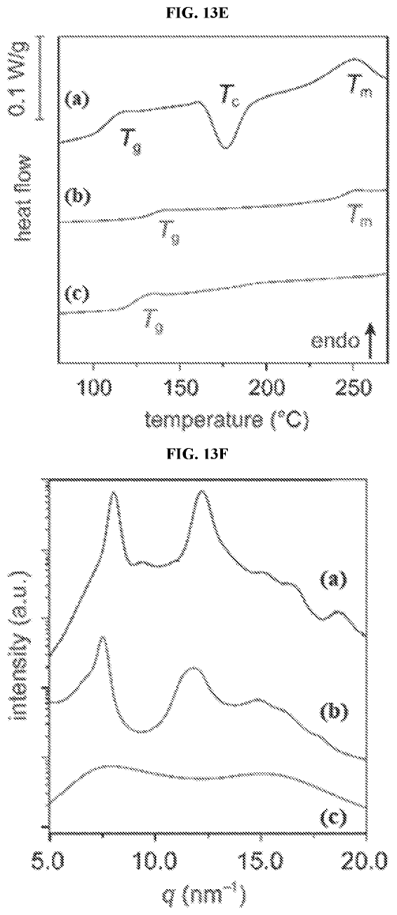

A series of LACoPs were synthesized as initiators for NN-dimethylacrylamide (DMAA) photopolymerizations under visible-light irradiation (FIGS. 13A-13B,). FIG. 13A summarizes the data of meso diad percentages obtained from LACoP-initiated polymerizations in methanol with varied loadings of lanthanum(III) trifluoromethanesulfonate (La(OTf)₃). The initial attempts involving 1b with an alkylated cyclen ligand resulted in polyDMAA with 75-85% m when 0.5-2.5 mol % La(OTf)₃ was employed relative to DMAA. The degree of isotacticity monotonically increased with increasing equivalents of La(OTf)₃. In comparison, a lower percentage of meso diads (<70% m) was obtained with 2.5 mol % La(OTf)₃ from a conventional RP initiated by a diphenyl(2,4,6-trimethylbenzoyl)phosphine oxide (TPO) photo-initiator.

An R—Co^{III}/por complex without tethered ACE (1a) provided up to 82% in even at a 5% La(OTf)₃ loading. Adding non-tethered ACE into the 1a-initiated polymerization resulted in negligible change in the stereocontrol, suggesting the essential role of the covalently tethered LA ligand in meso radical addition. Without wishing to be bound by theory, the slightly enhanced isotacticity compared to RP in 1a-initiated polymerizations could be attributed to the weak LA interaction with the carboxylate group of 1a as well as a controlled chain-growth process mediated by Co/por. Initiator 1c, comprising an LA ligand at the meta-position with respect to the porphyrin ring resulted in moderate enhancement in the isotacticity compared to 1b, which comprises para-substitution (e.g., 88% m (1c) vs 85% m (1b) at 2.5 mol % La(OTf)₃ loading.

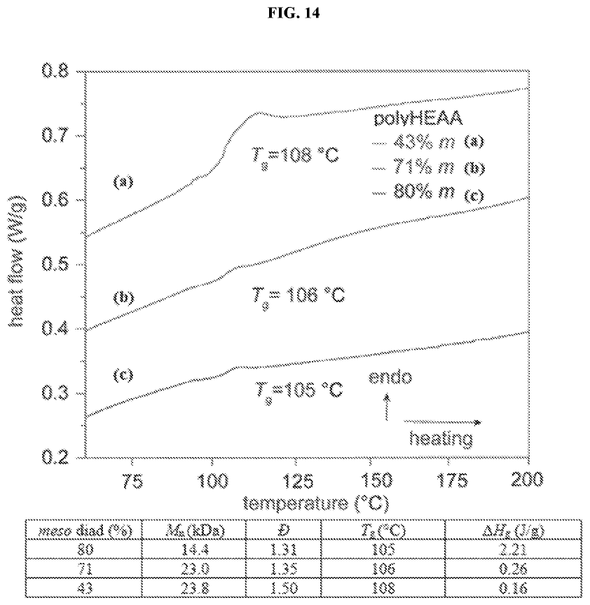

Further investigation of LACoPs was centered on engineering the binding affinity and geometry of the LA ligand. Co/por complex 1e, comprising a tridentate triazacyclononane-derived ligand was synthesized with a similar LA binding constant to the tetradentate ACE. PolyDMAA with over 90% in was obtained in a 1e-initiated polymerization with merely 2.5 mol % La(OTf)₃ added. The percentage of meso-meso triads (mm) in the polyDMAA with 95% in was quantified to be 90% mm according to its clearly distinguishable chemical shifts in the proton nuclear magnetic resonance (¹H-NMR) spectrum (FIG. 14). It has been hypothesized that the improved stereocontrol observed with 1e, as compared to 1c, plausibly originated from the enhanced interactions of 1e with chain-end pendants due to its reduced ligand cone angle and coordination number. In contrast, 1d resulted in poor stereocontrol due to the weak N-monosubstituted crown ether ligand with a binding constant roughly 8 orders of magnitude lower than cyclen.

In addition to the ligand design, selecting appropriate LA is equally critical to the stereocontrol, especially in a reaction medium rich in the aforementioned background chelating species. In the hypothesized mechanism, at least two coordination sites of the LA needs to be reserved for the chelation with chain-end pendant groups in a meso radical addition. Among a series of LAs screened, rare-earth metal cations with a large coordination number (e.g., lanthanum(III) (La³⁺) and yttrium(III) (Y³⁺) ions) presented overwhelmingly high performance in the stereocontrol.

The scope of monomers was expanded to acrylamides with various N-substituents (FIG. 13C). 1e-initiated polymerizations generated polymers with a higher isotacticity compared to TPO-initiated polymerizations from all monomers. Alkyl-substituted acrylamides, including N,N-diethylacrylamide (2b, DEAA) and N-isopropylacrylamide (2c, NIPAM), provide polymers with over 90% m with 5 mol % La(OTf). A wide range of polar pendants were well-tolerated, including monomers substituted with ether, amine, alcohol, and cationic moieties.

Polymerizations utilizing monomers 2d-2g resulted in polymers with isotacticity up to 85% m, slightly lower than those alkyl-substituted acrylamides due to the competitive coordination interactions of the pendant ether groups. N-(3-(dimethylamino)propyl)acrylamide (2h) and N-(2-hydroxyethyl)acrylamide (2i, HEAA) that are not compatible with other polymerization techniques were isotactically polymerized using 1e. Positively charged polymers with nearly 80% m were synthesized from (3-acrylamidopropyl)trimethylammonium trifluoromethanesulfonate (2j, APTMAT), while no isotacticity enrichment was observed in the 1e-initiatiated polymerization of zwitterionic monomer 2k, possibly due to the interference of strongly coordinating sulfonate anions.

The tacticity-varied polyDMAA (FIG. 13D) were further confirmed by their crystallinity and related thermal properties evaluated by differential scanning calorimetry (DSC) and wide-angle X-ray scattering (WAXS). An isotactic polyDMAA (95% m) exhibited a glass transition temperature (T_{g}) of 109° C., which is 20° C. lower than its atactic counterpart with 51% in (FIG. 13E). First-order phase transition absent in the atactic sample was observed in the DSC heating curve of the isotactic polyDMAA with an exothermic crystallization peak at T_{c} of 177° C. and an endothermic melting peak at Tₘ of 258° C., suggesting the formation of a semi-crystalline structure. Sharp and intensive diffractions originating from the crystalline phase were observed in WAXS profiles of the two isotactic polyacrylamides (FIG. 13F). The d-spacings were calculated from the scattering vector magnitudes (q=2π/d) to be 0.79 nm for polyDMAA with 95% m.

### Example 9: Kinetic and Mechanistic Studies of LACoP-Mediated LRP

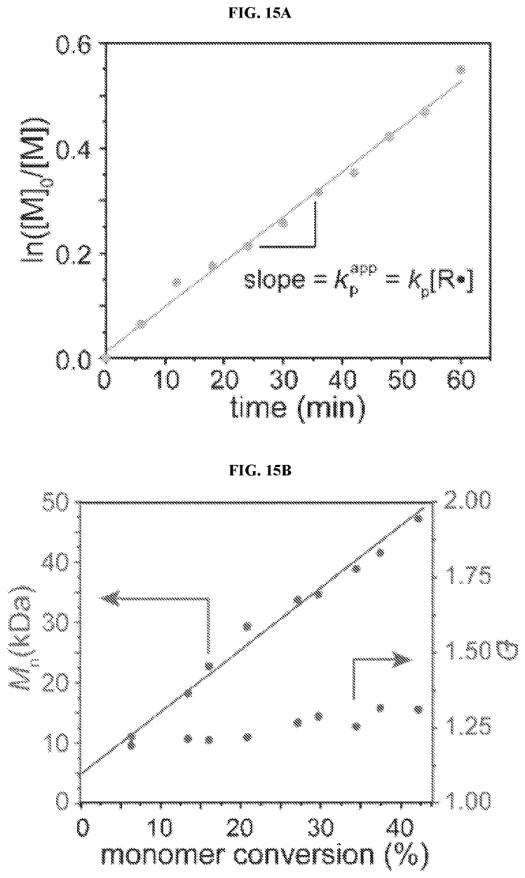

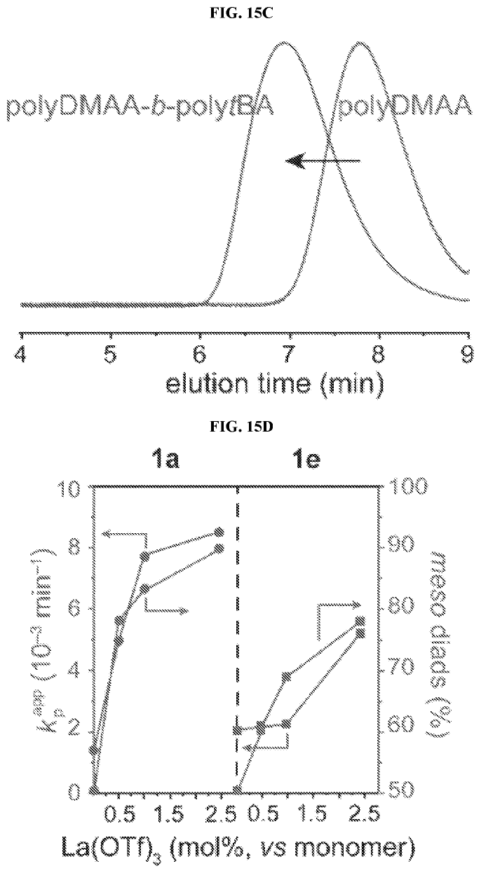

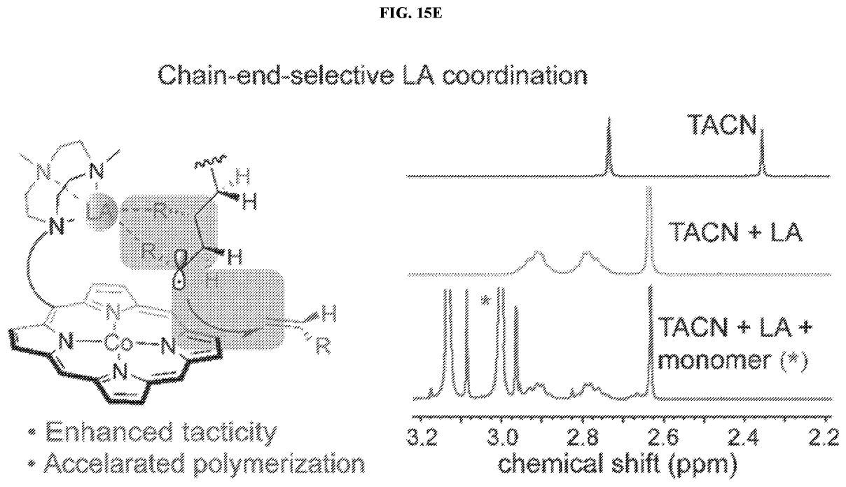

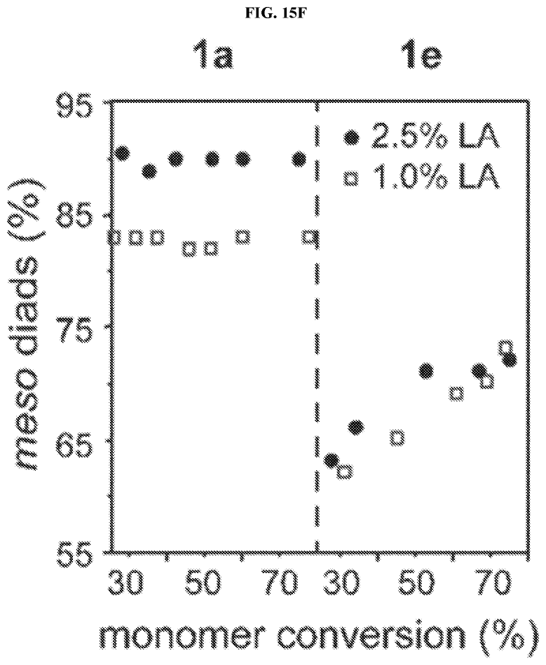

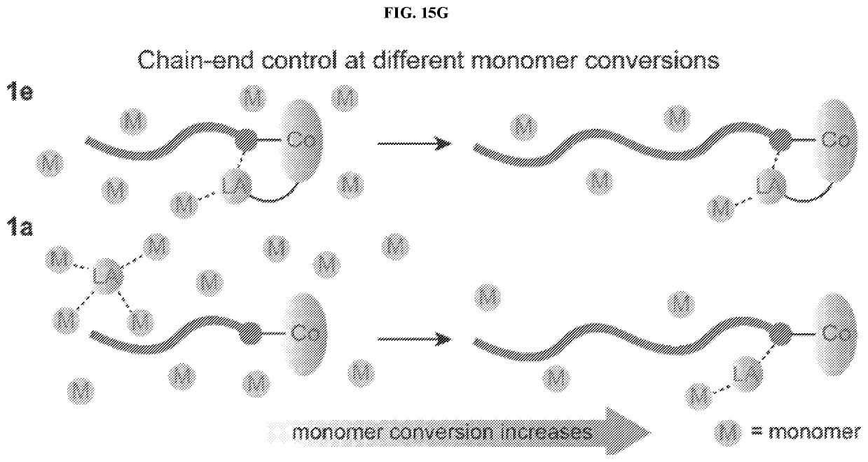

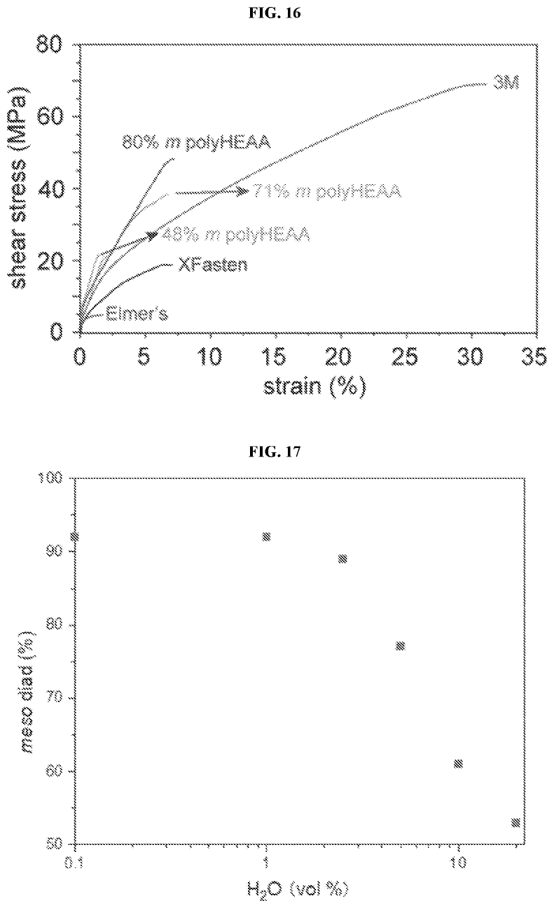

The characteristics of a living chain-growth process were probed in the 1e-initiated polymerizations of DMAA. The instantaneous quenching of the polymerization upon either addition of a nitroxide radical or removal of light irradiation (Example 3) validated the radical polymerization pathway in the presence of LA. The pseudo-first-order polymerization kinetics (FIG. 15A) resulted in narrowly distributed molecular weight (i.e., dispersity index Ð<1.3) that grew linearly with increasing the monomer conversion (FIG. 15B). The deviation of the molecular weight from a theoretical value originated from the relatively low initiation efficiency due to a larger bond dissociation energy of the Co-carbonyl bond of the initiator than the Co—C bond at the polyacrylamide chain end (FIG. 16 and Table 11).

TABLE 11
Adhesion test results of polyHEAA
with various degrees of tacticity

| meso diad | Mₙ | | interfacial toughness | fracture stress |
|---|---|---|---|---|
| (%) | (kDa) | Ð | (kJ/m²) | (MPa) |
| 80 | 14.4 | 1.31 | 4.1 | 21.3 |
| 71 | 23.0 | 1.35 | 33.7 | 37.9 |
| 43 | 23.8 | 1.50 | 42.4 | 42.4 |

The livingness rendered by the Co/por-mediated reversible radical deactivation was evidenced by the successful synthesis of polyDMAA-containing block copolymers. An isotactic polyDMAA macroinitiator end-functionalized with Co^{III}/por was synthesized and isolated from a 1e-initiated polymerization. The clear shift of the gel permeation chromatography (GPC) trace (FIG. 15C) upon chain extension of the macroinitiator with tert-butyl acrylate (tBA) suggested a high chain-end fidelity preserved in a living chain-growth process. This stereocontrolled LRP also opened a way to the one-pot synthesis of stereo-block polyacrylamides containing an atactic and an isotactic block by injecting LA solution at a given time to LACoP-mediated homopolymerizations (Example 4).

Systematic comparison of the kinetic characteristics under varied reaction conditions provided detailed mechanistic insights into the LACoP-mediated LRP. LA-accelerated polymerization that occurs in conventional RP was also observed in LACoP-mediated LRP (FIG. 15D). This acceleration at an unchanged radical concentration could be ascribed to the LA-enhanced reactivity of the propagating radical, which is in agreement with the postulated chain-end controlled meso radical addition. LA-induced acceleration in the LACoP-mediated LRP was investigated by relating both the apparent propagation rate coefficient

$$( {k}_{p}^{app} )$$

and degree of isotacticity with the La(OTf)₃ loading (FIG. 15D).

In the 1a-initiated LRP that involves weak LA chelation, La(OTf)₃ showed little impact on the polymerization rate or isotacticity until the amount of La(OTf)₃ reached 2.5 mol %. When 1e was instead employed, a sharp increase of

$${k}_{p}^{app}$$

with isotacticity as high as 83% in was obtained below 1 mol % La(OTf)₃. For further comparison,

$${k}_{p}^{app}$$

of 5.0×10⁻³ min⁻¹ with 78% in was achieved using 1e and only 0.5 mol % La(OTf)₃, while 2.5 mol % LA was needed in the 1a-initiated LRP to obtain a similar level of acceleration

$$( {k}_{p}^{app} = 5.4 \times {10}^{- 3} {min}^{- 1} )$$

and isotacticity (77% m). The remarkable acceleration accompanied with a more prominent isotacticity enhancement in 1e-initiated LRP demonstrated that the covalently attached ACE facilitated the chain-end specific LA chelation that is beneficial for both fast kinetics and meso radical addition.

The preferential LA coordination with ACE over other species (e.g., monomer, and solvent) was investigated by ¹H-NMR spectroscopy (FIG. 15E and FIG. 4). The almost identical NMR spectra of a La³⁺/triazacyclononane (TACN) solution before and after adding DMAA monomers indicated stable La³⁺/TACN complexation at a polymerization-relevant stoichiometry. Methanol was chosen as the solvent for the polymerization due to its excellent solubility for all reaction species and moderate binding affinity to La³⁺. When a solvent with stronger coordination ability (e.g., dimethylsulfoxide) was employed, the diminished chelation of La³⁺ with chain ends led to an atactic polymer. The good compatibility of LACoP with a protic environment allowed a stereocontrolled LRP of DMAA (ca. 90% m) in the presence of up to 2.5% water by volume (FIG. 17).

The quality of stereocontrol can be improved by suppressing other LA-chelating species that compete with the chain-end chelation. Therefore, in conventional RP and 1a-initiated LRP the degree of isotacticity changed with the progressive consumption of monomers due to the difference in binding strengths of monomer and polymer with LA. The conversion-independent isotacticity in 1e-initiated LRP (FIG. 15F) is consistent with the hypothesized LACoP-enabled stereocontrol. That is, LA is selectively chelated by the pendant groups located at the radical chain end throughout the entire polymerization (FIG. 15G). This chain-end control mechanism was corroborated with the diad/triad composition analysis based on Markovian statistics (Example 5).

### Example 10: Tacticity-Dependent Material Properties

### Thermo-Responsive Properties

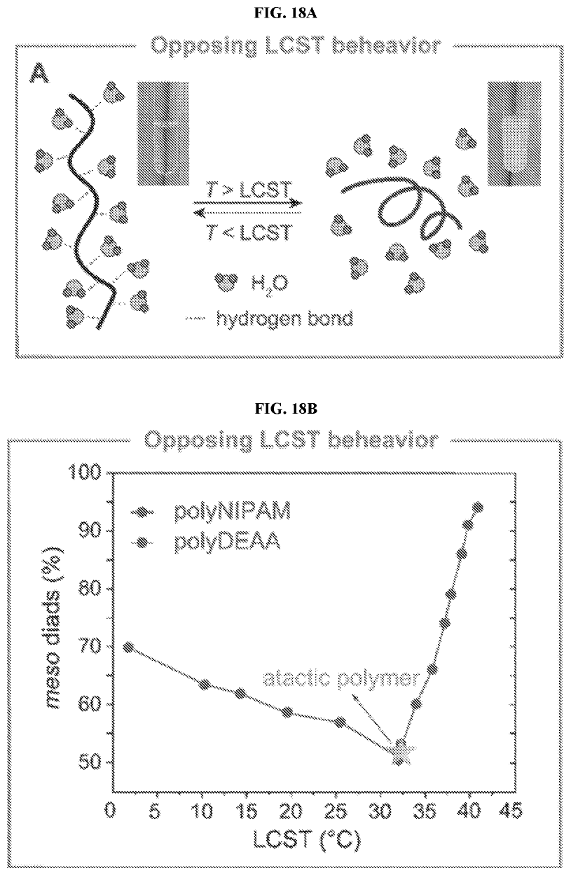

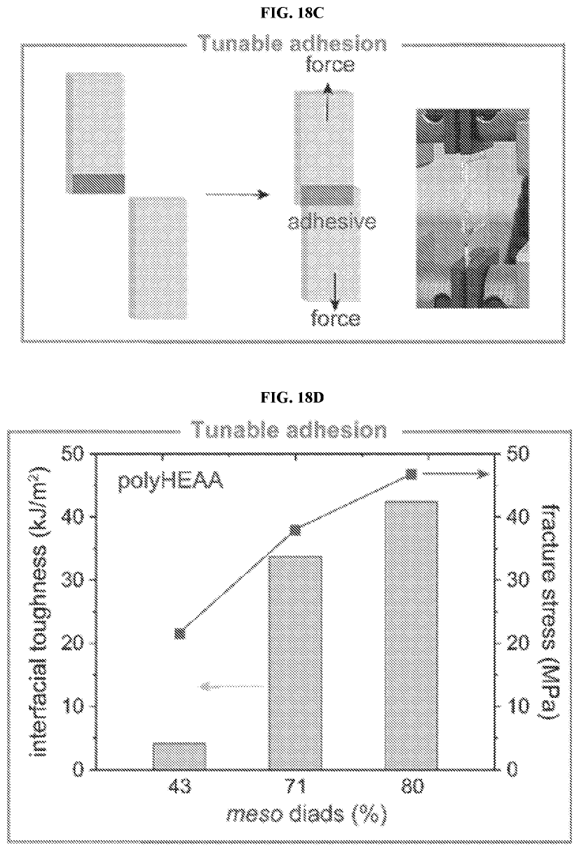

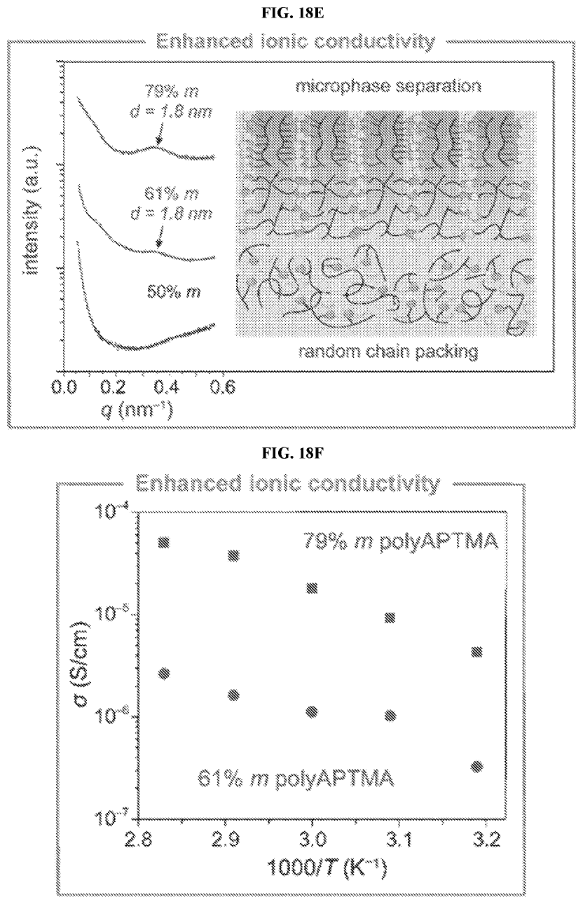

The stereocontrolled LRP provides an ideal platform for the preparation of well-defined polymers for accurate and systematic assessment of the impact of tacticity on polymers properties. Thermo-responsive properties that are widely utilized in biomedical engineering and design of smart devices were then investigated for isotacticity-enriched polyacrylamides. The thermo-responsive behaviors are captured by the lower critical solution temperature (LCST) above which a dissolved polymer becomes insoluble in water (FIG. 18A). The most commonly studied thermo-responsive polyacrylamides (i.e., polyDEAA and polyNIPAM) with an atactic microstructure, share a mutual LCST around 33±1° C. Such single-point LCST limits their broader applications and has to be adjusted by copolymerizing with additional comonomers. Nevertheless, this copolymerization strategy lacks robust predictability and reproducibility due to the unclearly defined monomer sequence as well as the underexplored influence of molecular weight and composition on LCST.

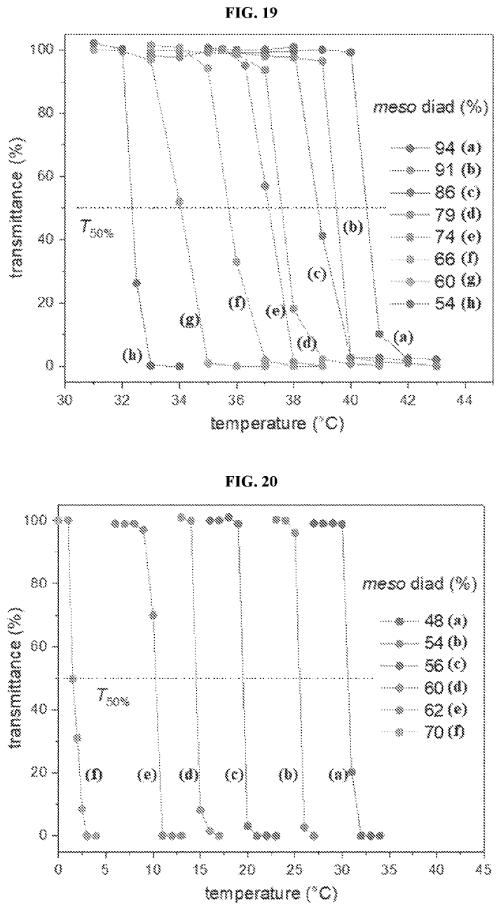

A significantly expanded tuning window of LCST of polyacrylamides was assessed herein by varying the degree of isotacticity. The LCST was determined by UV-vis spectroscopy where a sharp drop of the transmittance occurred in the programmed heating experiment (FIGS. 19-20 and Table 12). LCST increased with increasing isotacticity of polyDEAA, plausibly due to the enhanced hydrophilicity through cooperative hydrogen bond interactions between the meso-configurated neighboring units and a water molecule (FIG. 18B).

TABLE 12
LCSTs of polyDEAA and polyNIPAM

| Polymer | Mₙ (kDa) | Ð | LCST (° C.) |
|---|---|---|---|
| polyDEAA-94% m | 52.3 | 1.33 | 40.9 |
| polyDEAA-91% m | 48.9 | 1.31 | 39.8 |
| polyDEAA-86% m | 47.4 | 1.22 | 39.1 |
| polyDEAA-79% m | 55.3 | 1.25 | 37.9 |
| polyDEAA-74% m | 78.2 | 1.49 | 37.2 |
| polyDEAA-66% m | 33.9 | 1.37 | 35.8 |
| polyDEAA-60% m | 41.5 | 1.32 | 34 |
| polyDEAA-54% m | 48.9 | 1.31 | 32.3 |
| polyNIPAM-48% m | 29.5 | 1.46 | 32.0 |
| polyNIPAM-54% m | 35.2 | 1.41 | 25.5 |
| polyNIPAM-56% m | 44.4 | 1.55 | 19.5 |
| polyNIPAM-60% m | 39.3 | 1.47 | 14.4 |
| polyNIPAM-62% m | 55.1 | 1.44 | 10.3 |
| polyNIPAM-70% m | 47.9 | 1.50 | 1.5 |

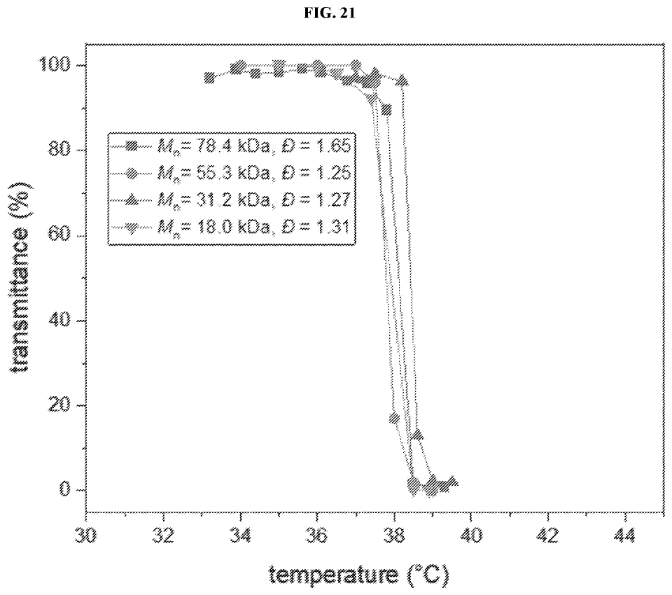

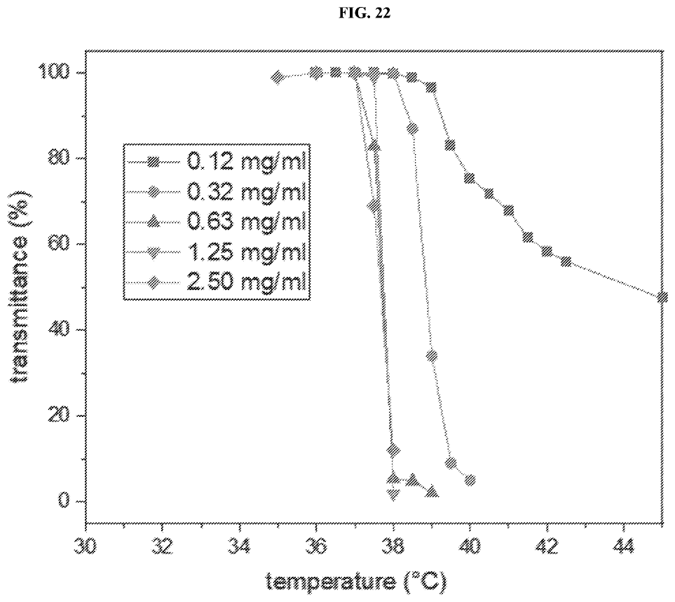

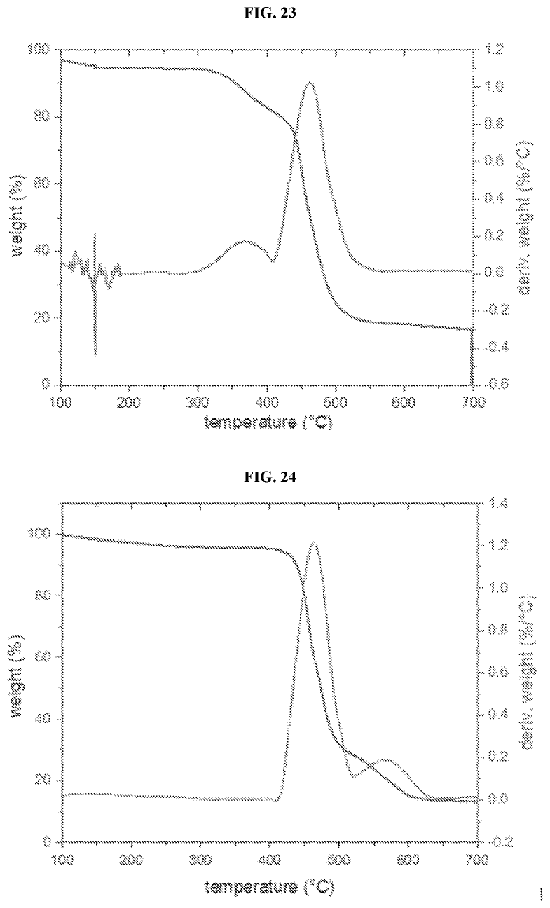

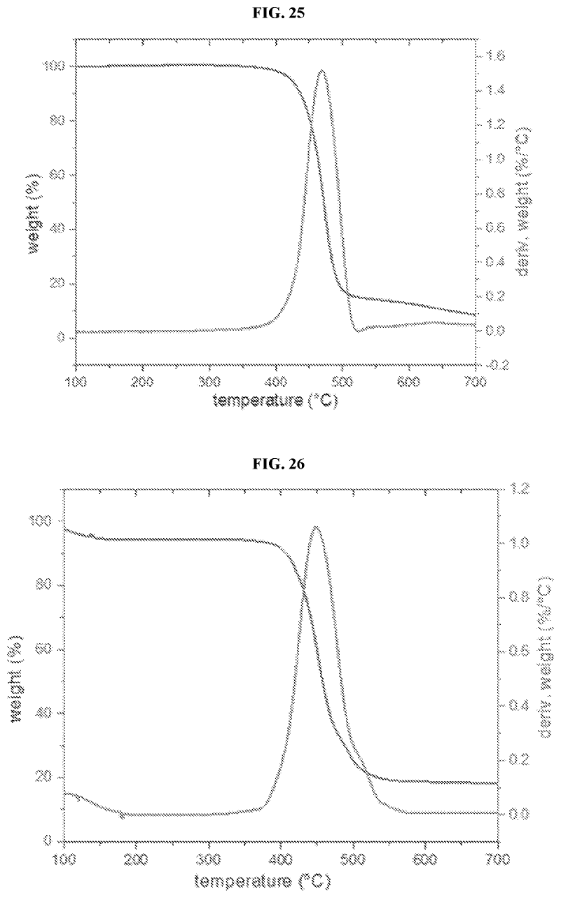

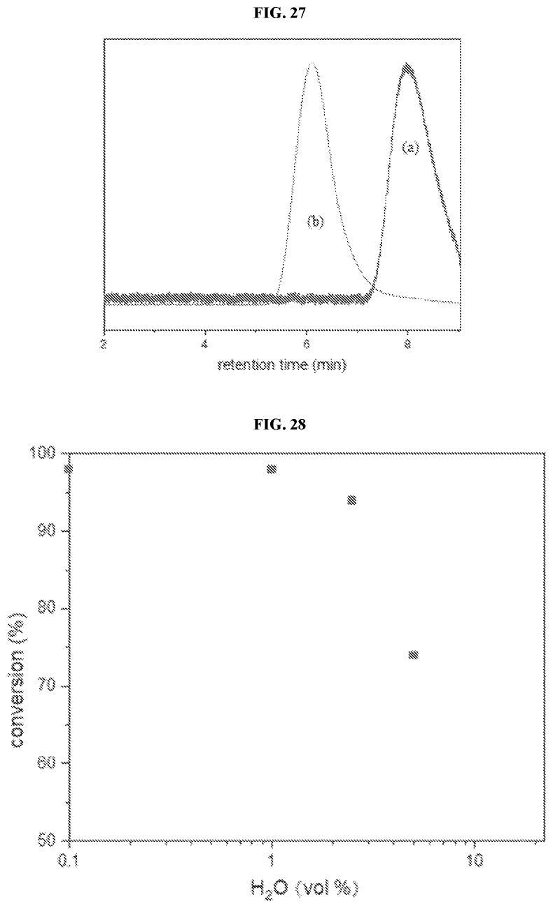

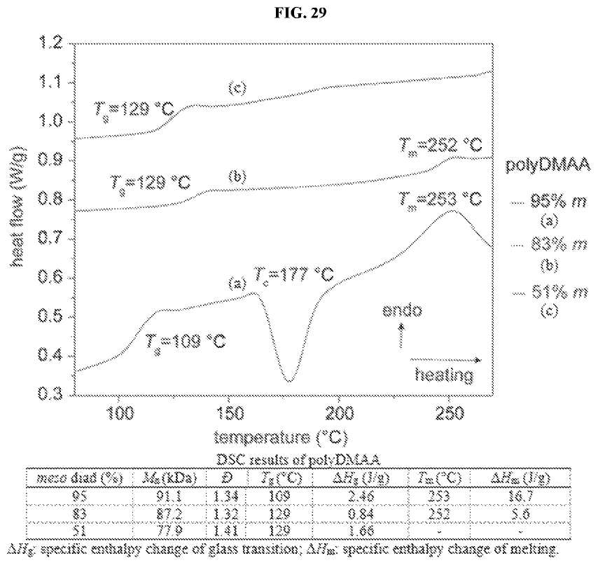

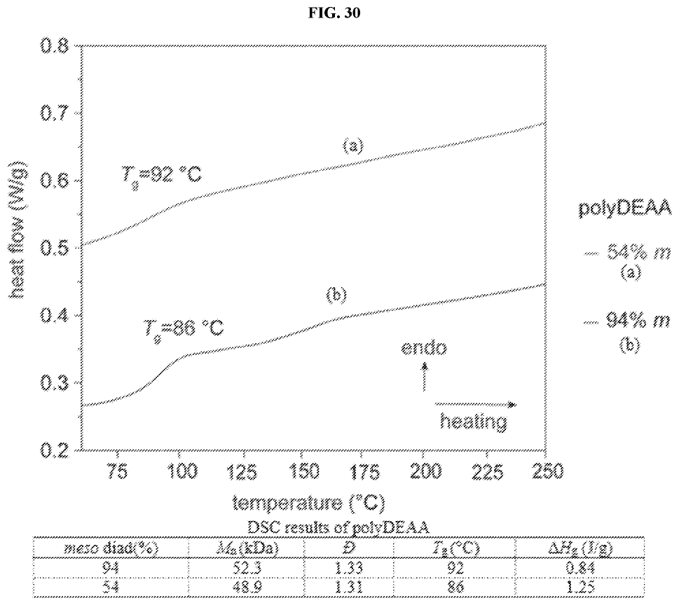

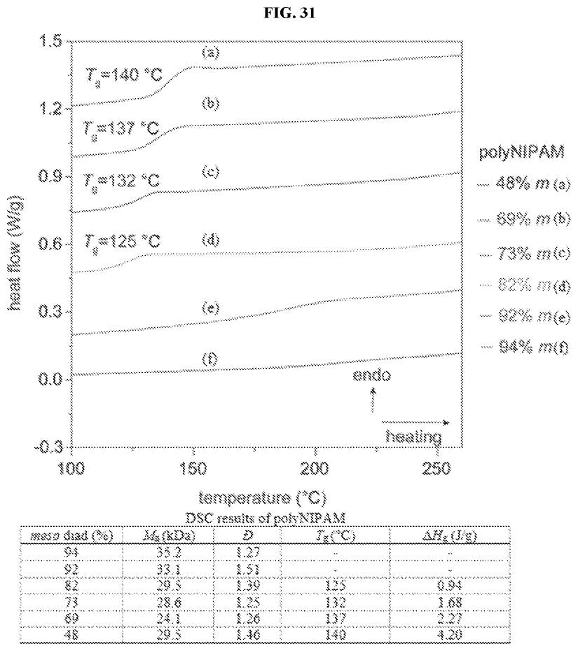

When the percentage of meso diads changed from 66% m to 94% m, the LCST increased from 35.8° C. to 40.9° C. Overall, a continuous change of LCST from 33° C. to 42° C. was achieved in homopolymerized DEAA without compositional variance. The LCST under the studied conditions was found independent of molecular weight and concentration of polyDEAA (FIGS. 21-22). Thus, a one-to-one correspondence was constructed between the degree of isotacticity and LCST.

Different from the LCST mechanism of polyDEAA, the pendant group of polyNIPAM serves a dual role of both hydrogen bond donor and acceptor. Adjacent pendant units of polyNIPAM with a meso configuration introduce enhanced intramolecular interaction and therefore increase the hydrophobicity of the polymer. Hence, an opposite tacticity-dependence was realized in the LCST of polyNIPAM (FIG. 18B). The aqueous solution of polyNIPAM with 54% m became cloudy at 25.5° C. and further increasing to 70% m resulted in a sub-room-temperature thermo-response at 1.5° C. The two different tacticity-dependences complementarily provide tunable LCST from 0° C. to 43° C., broadly covering the temperature range most relevant and interesting to biomedical studies and applications.

### Adhesive Properties

Further attempts toward property diversification was focused on the adhesion behaviors of polyHEAA, the atactic forms and analogues of which have demonstrated promise as bio-adhesives. Lap shear tests were carried out using glass slides adhered with isotacticity-varied polyHEAA containing glycerol plasticizers of a constant fraction (FIG. 18C). While the adhesion abilities of all polyHEAA samples were quantified to be within the anticipated window of commercial adhesives (FIG. 16), a monotonic increasing trend with respect to the degree of isotacticity was observed in both the failure shear stress and the interfacial toughness (FIG. 18D, Table 13). Isotactic polyHEAA with 80% m cost over 10 times higher energy than polyHEAA with 43% m to unbond the single-lap joint. This efficient isotacticity-enhancing adhesion without changes in chemical composition plausibly benefited from the meso-configurated neighboring hydroxy pendants that provided uniformly oriented hydrogen bonds to cooperatively adhere the glass surface.

TABLE 13
DSC traces of polyHEAA with various degrees of tacticity

| meso diad (%) | Mₙ (kDa) | Ð | T_{g} (°C.) | ΔH_{g} (J/g) |
|---|---|---|---|---|
| 80 | 14.4 | 1.31 | 105 | 2.21 |
| 71 | 23.0 | 1.35 | 106 | 0.26 |
| 43 | 23.8 | 1.50 | 108 | 0.16 |

### Solid-State Polyelectrolyte Properties

Design of high-performance solid-state polyelectrolytes is of high industrial value in battery and ion-exchange membrane applications. The conventional strategy of introducing additives into polyelectrolyte to enhance ionic conductivity often compromises other critical material properties such as processability, durability, and even the solid-state nature.

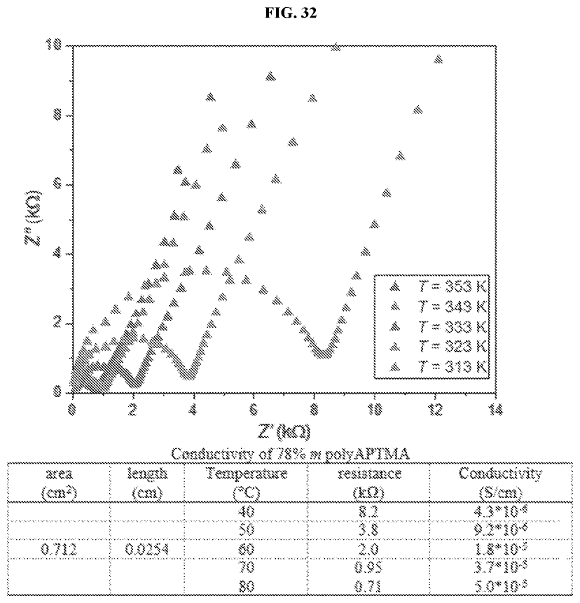

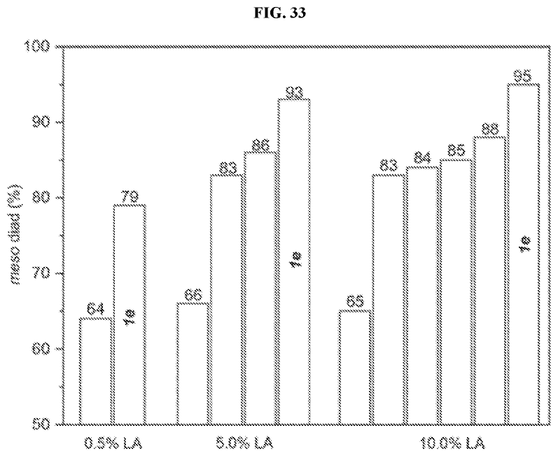

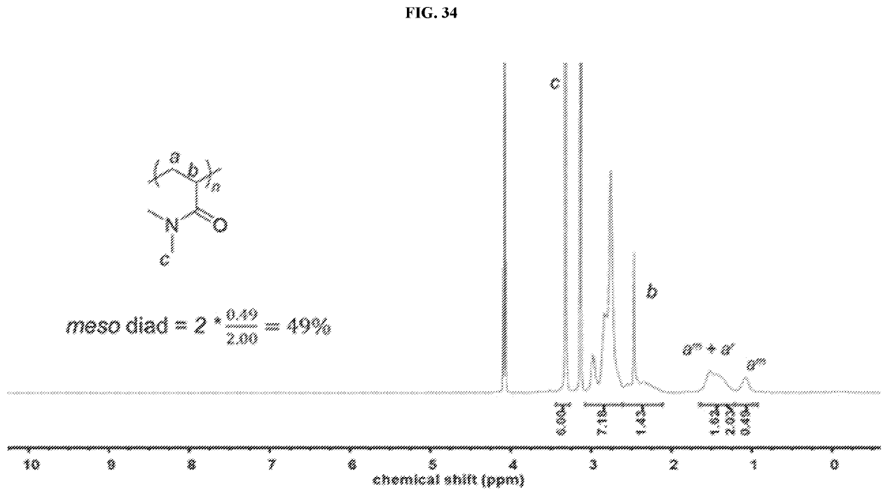

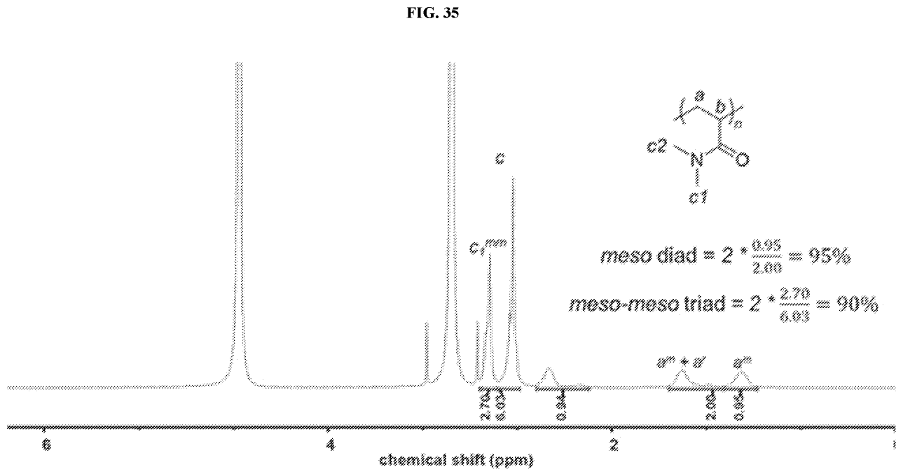

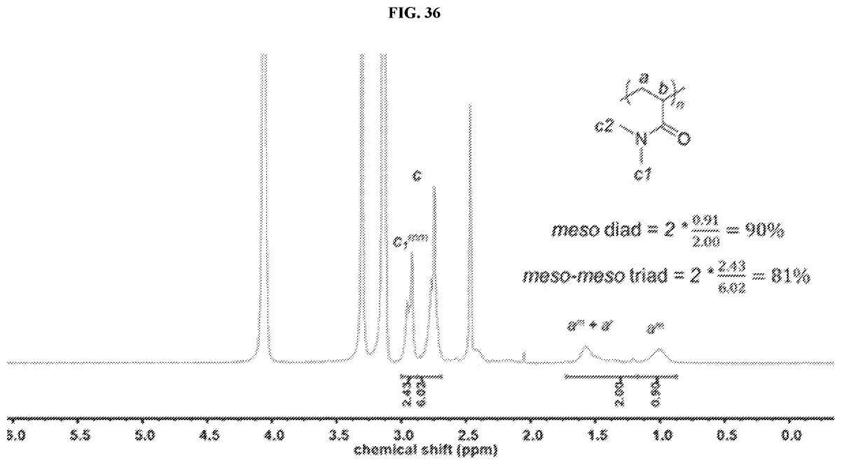

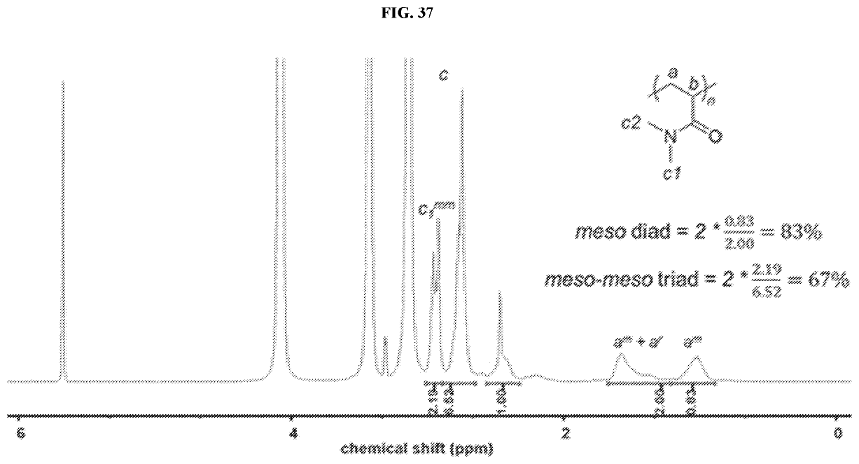

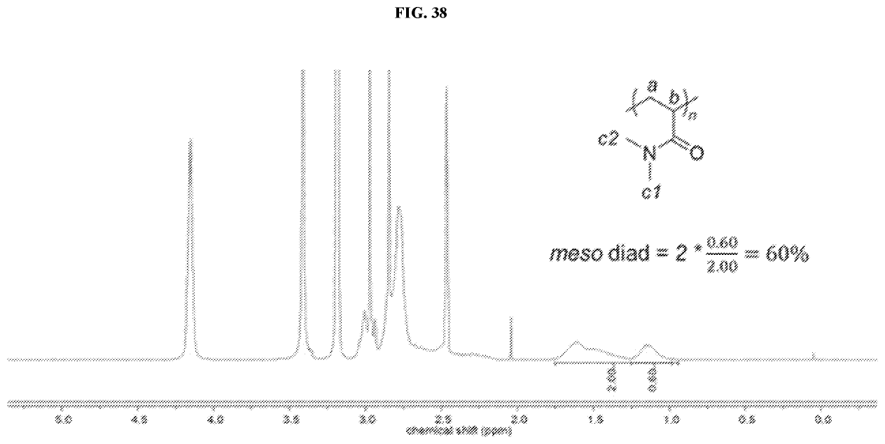

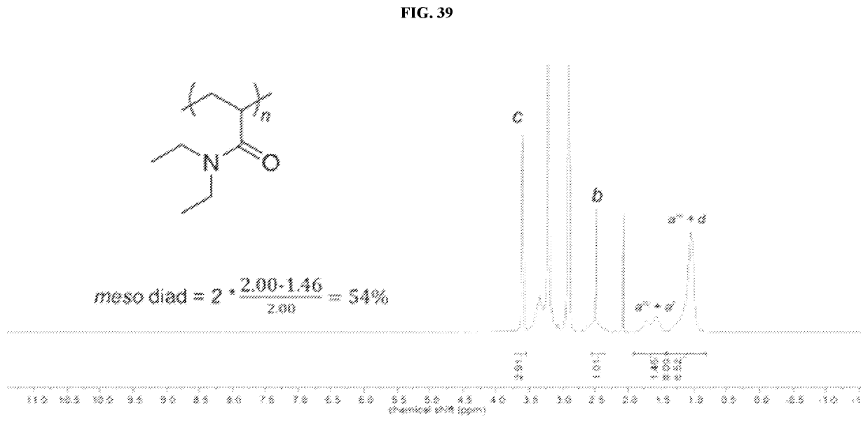

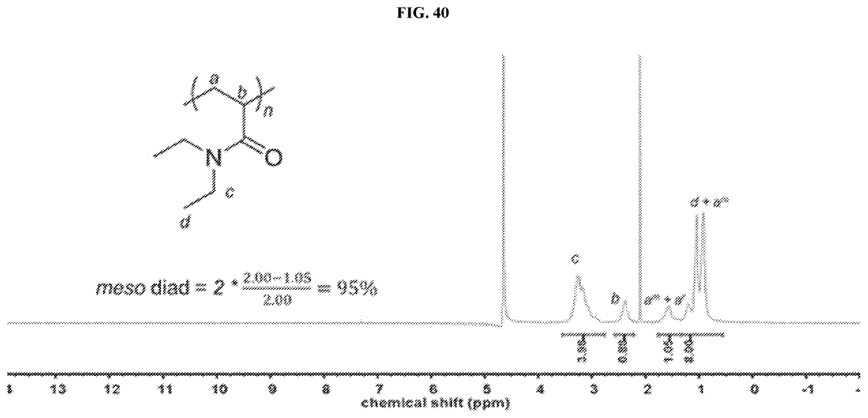

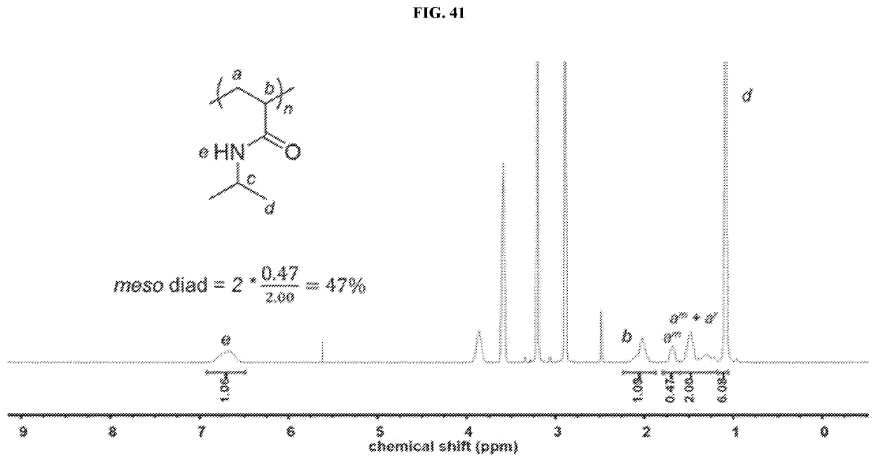

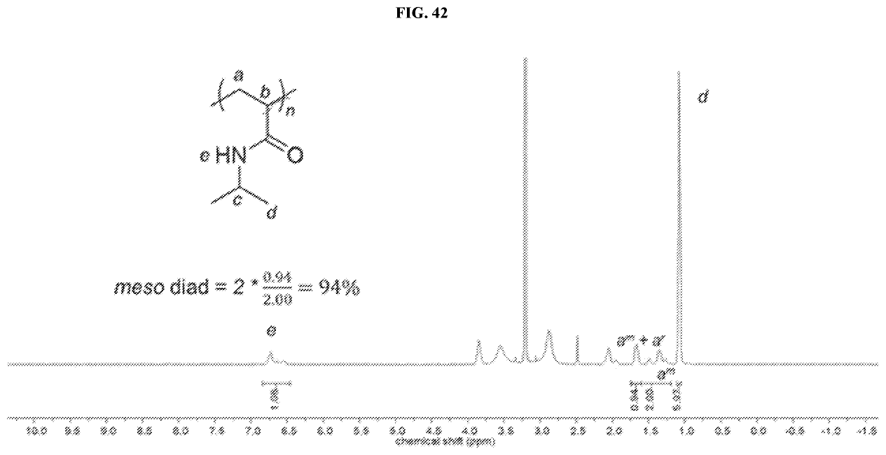

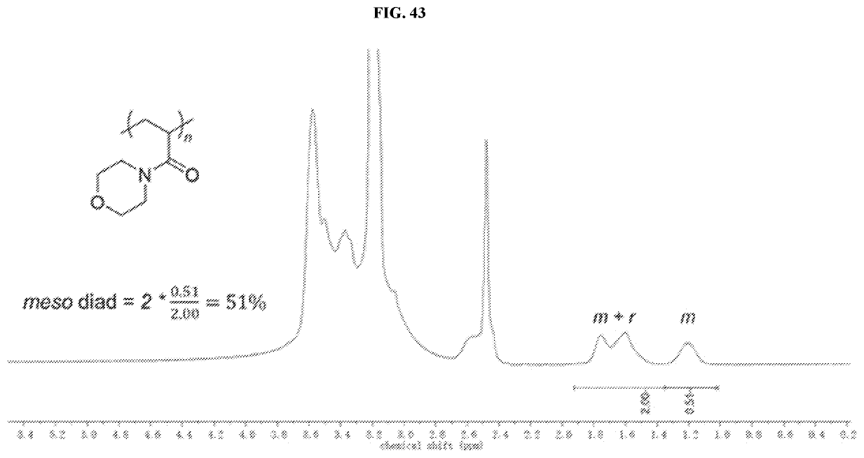

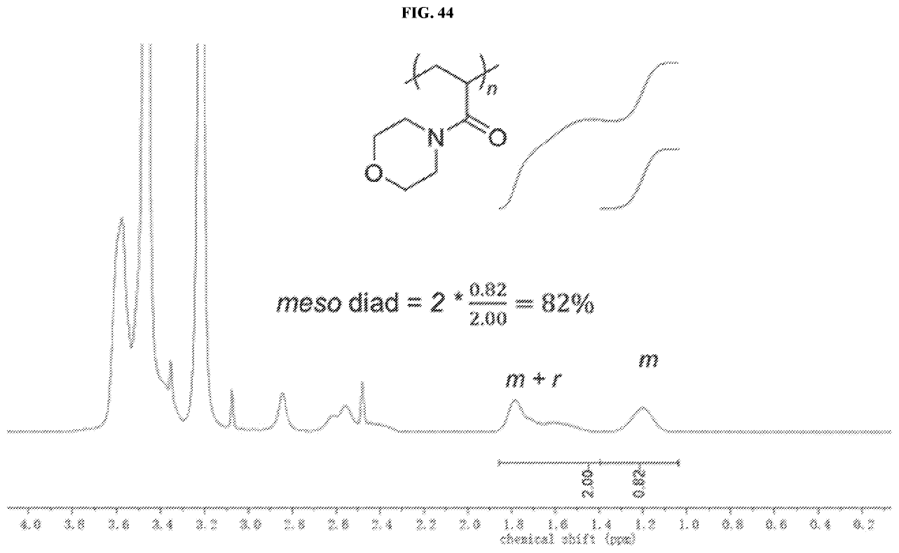

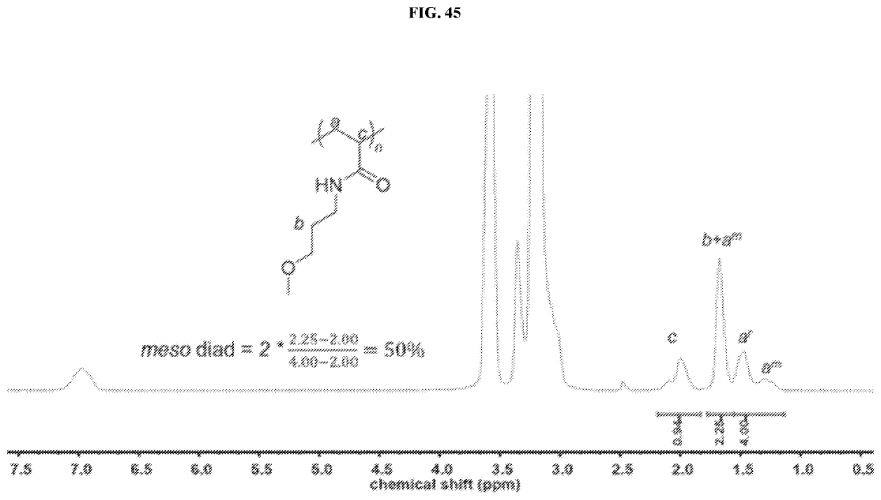

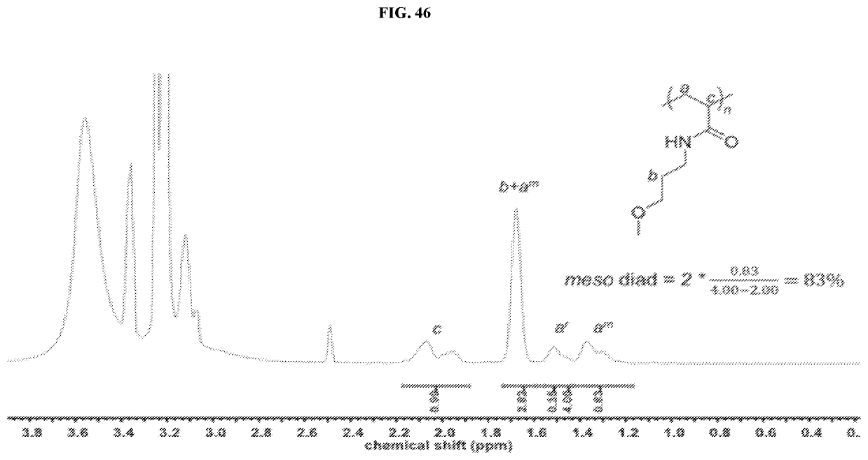

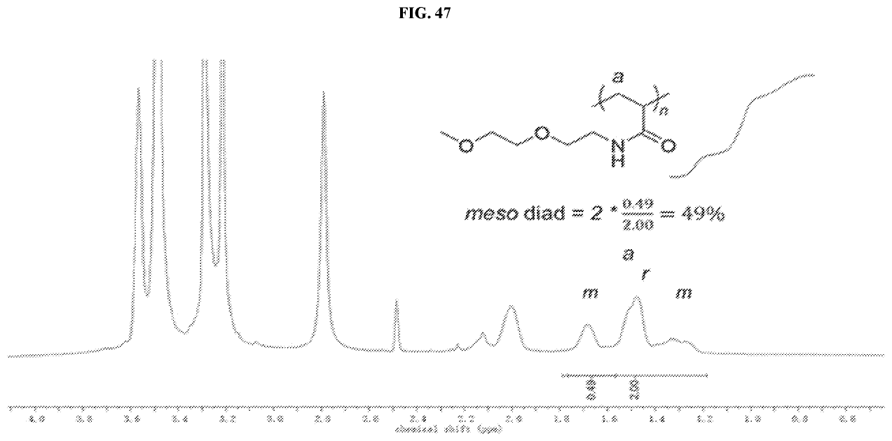

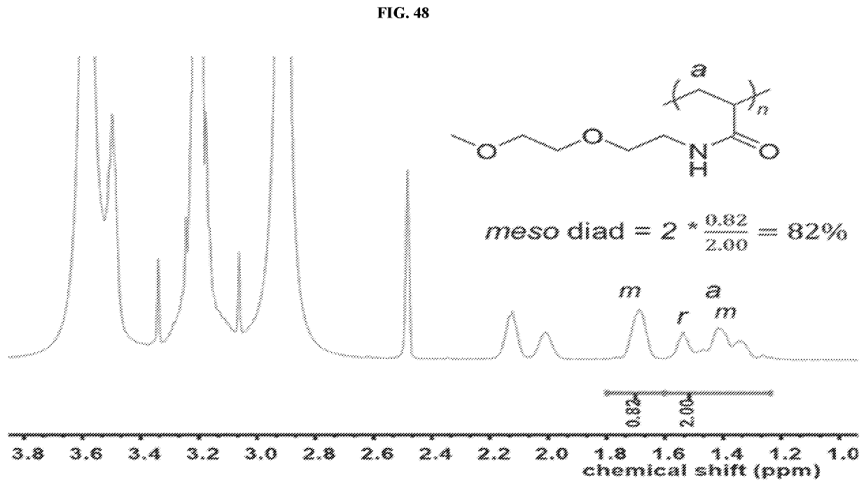

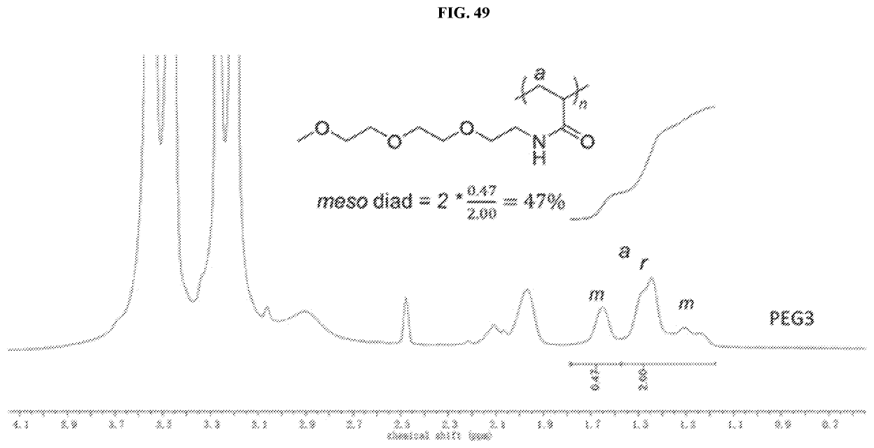

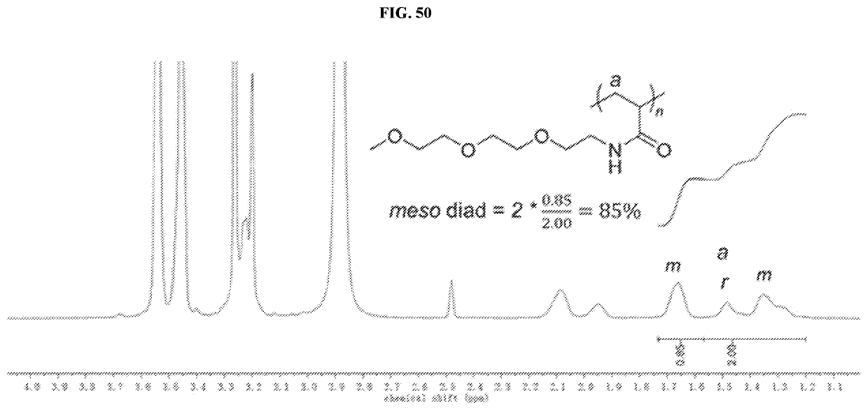

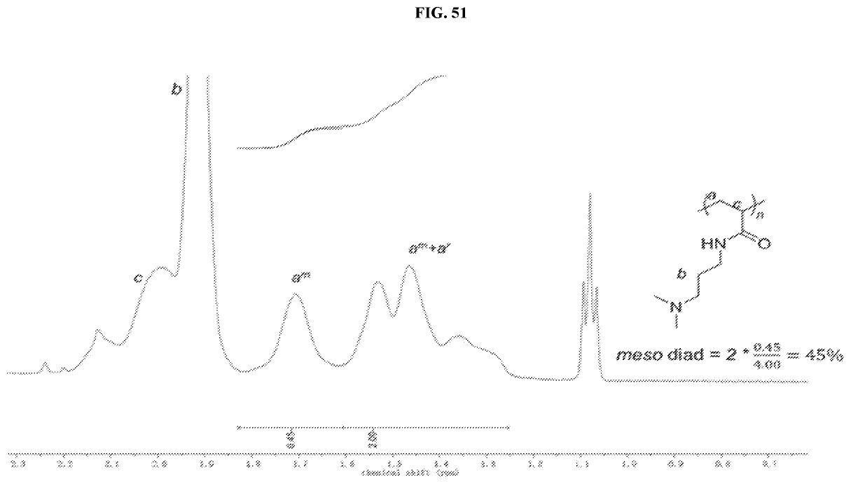

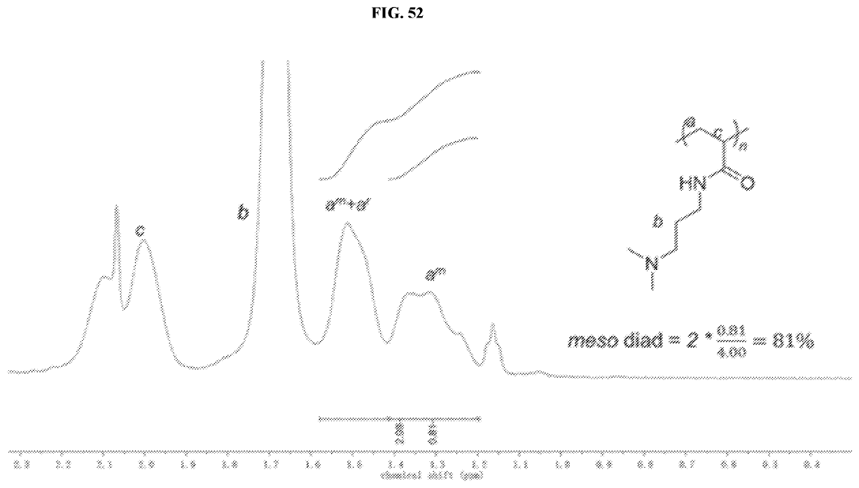

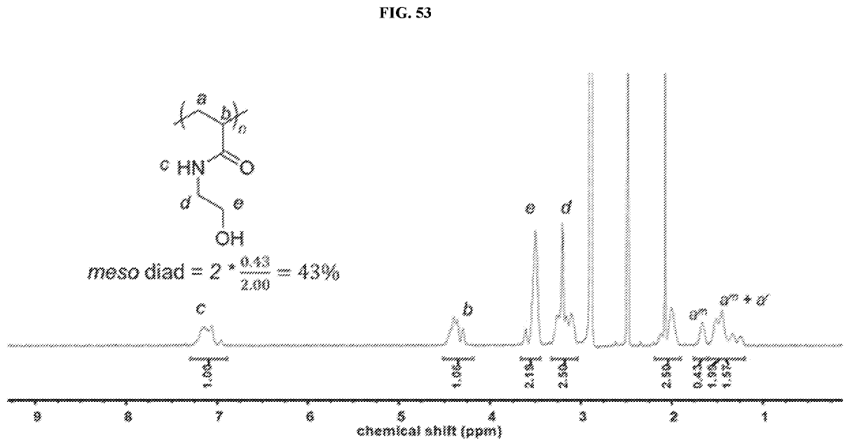

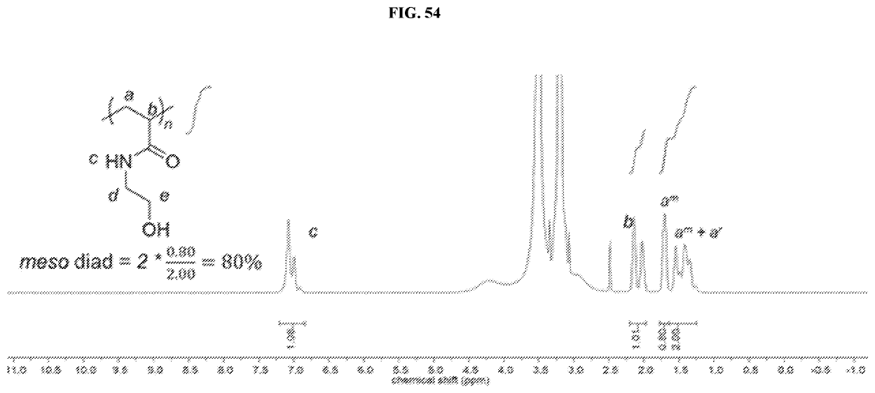

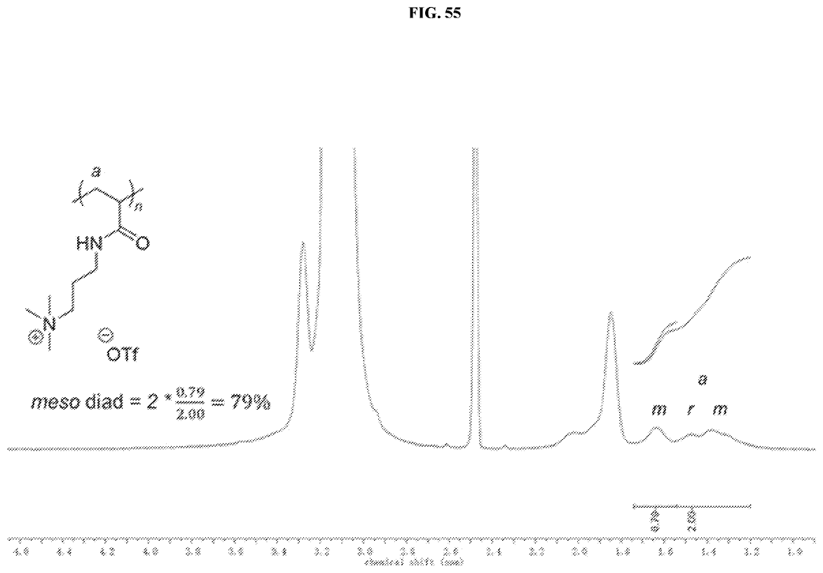

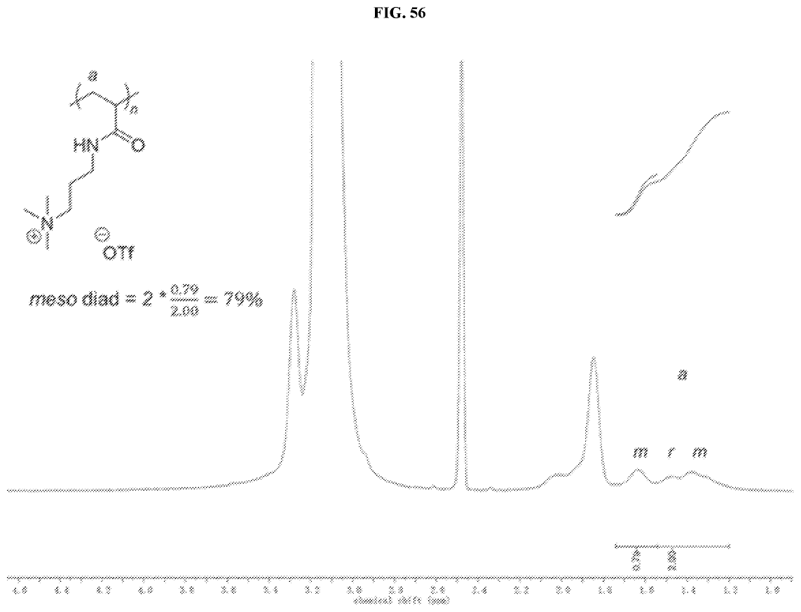

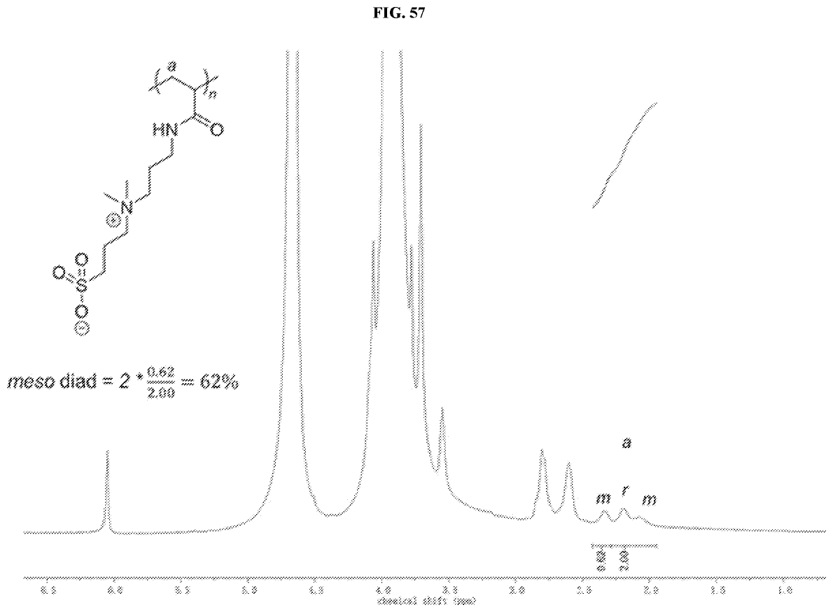

Demonstrated herein is an additive-free method to enhance ionic conductivity in the polyAPTMAT system that contains polycationic backbone and mobile counter anions. Three polyAPTMAT solids with similar shear moduli (xxxG′) were prepared with adjusted tacticity through LACoP-mediated LRP. A microphase-separated morphology with a correlation length of 1.8 nm was observed in isotacticity-enriched samples (FIG. 18E), indicting an ion clustering and related phase segregation facilitated by meso-configurated ionic pendants. The conductivity of atactic polyAPTMAT with randomly packed chains was too low to be detectable based on the employed electrochemical impendence spectroscopy (FIG. 32) method.

However, ion conductivity in the range of 10⁻⁷-10⁻⁴ S/cm was obtained in both isotactic samples. PolyAPTMAT with 79% meso diads and a more intense scattering peak exhibited ionic conductivity over 10 times higher than that with 61% meso diads, suggesting that the formed nanophase-separated structures provide conducting channels for the charge carries and therefore reduce the energetic barrier of the anion transport.

### ENUMERATED EMBODIMENTS

The following exemplary embodiments are provided, the numbering of which is not to be construed as designating levels of importance:

Embodiment 1 provides a compound selected from the group consisting of:

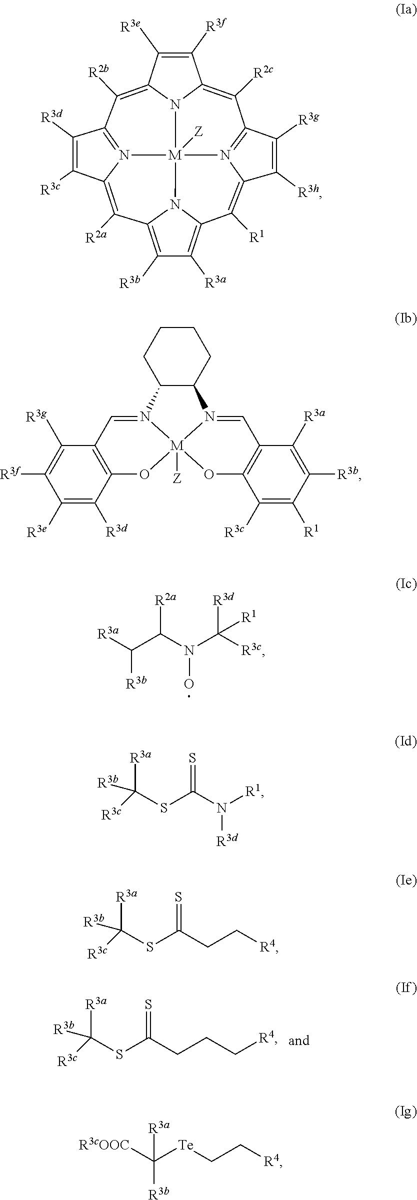

or a salt, solvate, stereoisomer, or isotopologue thereof, wherein:
M, if present, is Co;
Z is absent or an anionic ligand;
R¹, if present, is

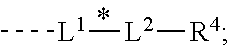

R^{2a}, R^{2b}, and R^{2c}, if present, are each independently selected from the group consisting of H, optionally substituted C₁-C₆ alkyl, optionally substituted C₃-C₈ cycloalkyl, optionally substituted C₆-C₁₀ aryl and optionally substituted C₂-C₁₀ heteroaryl;
R^{3a}, R^{3b}, R^{3c}, R^{3d}, R^{3e}, R^{3f}, R^{3g}, and R^{3h}, if present, are each independently selected from the group consisting of H and optionally substituted C₁-C₆ alkyl;
L¹, if present, is selected from the group consisting of -(optionally substituted C₆-C₁₀ arylenyl)-* and -(optionally substituted C₂-C₁₀ heteroarylenyl)-*,
   wherein a substituent of the C₆-C₁₀ arylenyl or the C₂-C₁₀ heteroarylenyl in L¹ can combine with a substituent of the C₆-C₁₀ aryl or C₂-C₁₀ heteroaryl in R^{2b} to form an optionally substituted C₂₀-C₃₀ heterocycloalkenyl;
L², if present, is selected from the group consisting of *—C(═O)N(R⁵)(optionally substituted C₁-C₆ alkylenyl)-, *—C(═O)O(optionally substituted C₁-C₆ alkylenyl)-, *—C(═O)(optionally substituted C₁-C₆ alkylenyl)-, *-(optionally substituted C₁-C₆ alkylenyl)-, *—N(R⁵)C(═O)(optionally substituted C₁-C₆ alkylenyl)-, *—OC(═O)(optionally substituted C₁-C₆ alkylenyl)-, *—C(═O)N(R⁵)(optionally substituted C₁-C₆ heteroalkylenyl)-, *—C(═O)O(optionally substituted C₁-C₆ heteroalkylenyl)-, *—C(═O)(optionally substituted C₁-C₆ heteroalkylenyl)-, *-(optionally substituted C₁-C₆ heteroalkylenyl)-, *—N(R⁵)C(═O)(optionally substituted C₁-C₆ heteroalkylenyl)-, and *—OC(═O)(optionally substituted C₁-C₆ heteroalkylenyl)-;
R⁴ is an optionally substituted C₆-C₈ heterocycloalkyl,
   wherein the C₆-C₈ heterocycloalkyl comprises at least three atoms selected from the group consisting of N, O, and S; and
each occurrence of R⁵, if present, is independently selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

Embodiment 2 provides the compound of Embodiment 1, wherein L¹ is selected from the group consisting of:

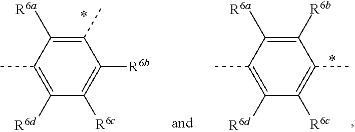

wherein R^{6a}, R^{6b}, R^{6c}, and R^{6d} are each independently selected from the group consisting of H, optionally substituted C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵.

Embodiment 3 provides the compound of Embodiment 2, wherein at least one of the following applies:
(a) at least one of R^{6a}, R^{6b}, R^{6c}, and R^{6d} is H;
(b) at least two of R^{6a}, R^{6b}, R^{6c}, and R^{6d} are H;
(c) at least three of R^{6a}, R^{6b}, R^{6c}, and R^{6d} are H; and
(d) each of R^{6a}, R^{6b}, R^{6c}, and R^{6d} are H.

Embodiment 4 provides the compound of any one of Embodiments 1-3, wherein L² is *—C(═O)NH(C₁-C₆ alkylenyl)-.

Embodiment 5 provides the compound of any one of Embodiments 1-4, wherein L² is *—C(═O)NHCH₂CH₂—.

Embodiment 6 provides the compound of any one of Embodiments 1-5, wherein R⁴ is:

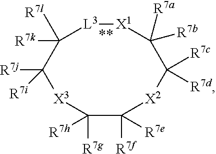

wherein:
L³ is selected from the group consisting of a bond and —X⁴—C(R^{7m})(R⁷ⁿ)—C(R^{7o})(R^{7p})—**;
X¹, X², X³, and X⁴, if present, are each independently selected from the group consisting of N(R⁸) and O;
R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, if present, are each independently selected from the group consisting of H, C₁-C₆ alkyl, CN, NO₂, OR⁵,
N(R⁵)(R⁵), halogen, C(═O)OR⁵, C(═O)N(R⁵)(R⁵), C(═O)R⁵, OC(═O)R⁵, and N(R⁵)C(═O)R⁵,
   wherein one of R^{7a}, R^{7b}, R^{7c}, R^{7d}, R^{7e}, R^{7f}, R^{7g}, R^{7h}, R⁷ⁱ, R^{7j}, R^{7k}, and R^{7l} is -L²-*, or
   one of X¹, X², and X³ is N-L²-*; and
R¹ is selected from the group consisting of H and optionally substituted C₁-C₆ alkyl.

Embodiment 7 provides the compound of any one of Embodiments 1-6, wherein one of X¹, X², and X³, is N-L²-*.

Embodiment 8 provides the compound of any one of Embodiments 1-7, wherein at least one of the following applies:
(a) at least one of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, is H;
(b) at least two of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(c) at least three of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(d) at least four of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(e) at least five of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(f) at least six of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(g) at least seven of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(h) at least eight of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(i) at least nine of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(j) at least ten of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(k) at least eleven of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(1) at least twelve of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(m) at least thirteen of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(n) at least fourteen of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H;
(o) at least fifteen of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H; and
(p) each of R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, are H.

Embodiment 9 provides the compound of any one of Embodiments 1-8, wherein R⁴ is selected from the group consisting of

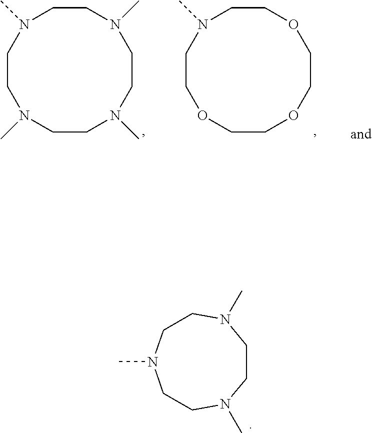

Embodiment 10 provides the compound of any one of Embodiments 1-9, wherein R^{2a}, R^{2b}, and R^{2d} are each independently:

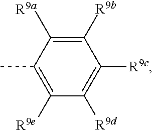

wherein R^{9a}, R^{9b}, R^{9c}, R^{9d}, and R^{9e} are each independently selected from the group consisting of H, optionally substituted C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵.

Embodiment 11 provides the compound of Embodiment 10, wherein R^{9a}, R^{9b}, R^{9e}, R^{9d}, and R^{9e} are each independently selected from the group consisting of H and CH₃.

Embodiment 12 provides the compound of any one of Embodiments 1-11, wherein
R^{2a}, R^{2b}, and R^{2e} are each independently

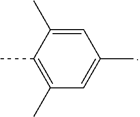

Embodiment 13 provides the compound of any one of Embodiments 1-12, wherein at least one of the following applies:
(a) at least one of R^{3a}, R^{3b}, R^{3c}, R^{3d}, R^{3e}, R^{3f}, R^{3g}, and R^{3h} is H;
(b) at least two of R^{3a}, R^{3b}, R^{3c}, R^{3d}, R^{3e}, R^{3f}, R^{3g}, and R^{3h} are H;
(c) at least three of R^{3a}, R^{3b}, R^{3c}, R^{3d}, R^{3e}, R^{3f}, R^{3g}, and R^{3h} are H;
(d) at least four of R^{3a}, R^{3b}, R^{3c}, R^{3d}, R^{3e}, R^{3f}, R^{3g}, and R^{3h} are H;
(e) at least five of R^{3a}, R^{3b}, R^{3c}, R^{3d}, R^{3e}, R^{3f}, R^{3g}, and R^{3h} are H;
(f) at least six of R^{3a}, R^{3b}, R^{3c}, R^{3d}, R^{3e}, R^{3f}, R^{3g}, and R^{3h} are H;
(g) at least seven of R^{3a}, R^{3b}, R^{3c}, R^{3d}, R^{3e}, R^{3f}, R^{3g}, and R^{3h} are H; and
(h) each of R^{3a}, R^{3b}, R^{3c}, R^{3d}, R^{3e}, R^{3f}, R^{3g}, and R^{3h} are H.

Embodiment 14 provides the compound of any one of Embodiments 1-13, wherein Z is absent.

Embodiment 15 provides the compound of any one of Embodiments 1-14, which is selected from the group consisting of

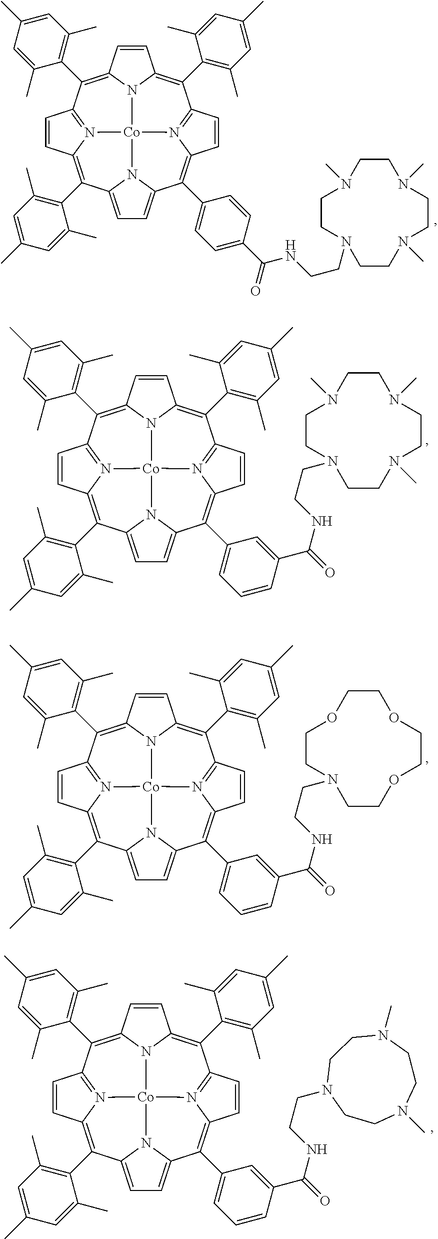

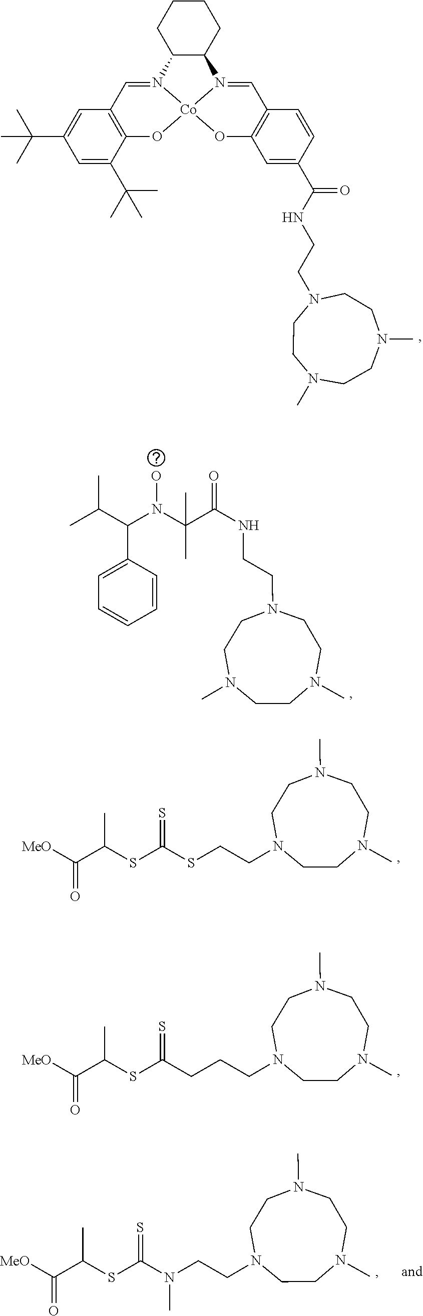

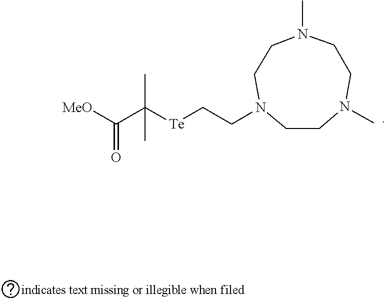

Embodiment 16 provides the compound of any one of Embodiments 1-13, wherein Z is selected from the group consisting of C(═O)OR⁵, optionally substituted alkyl, and halogen.

Embodiment 17 provides the compound of any one of Embodiments 1-13 and 16, wherein Z is C(═O)OMe.

Embodiment 18 provides the compound of any one of Embodiments 1-13 and 16-17, which is selected from the group consisting of:

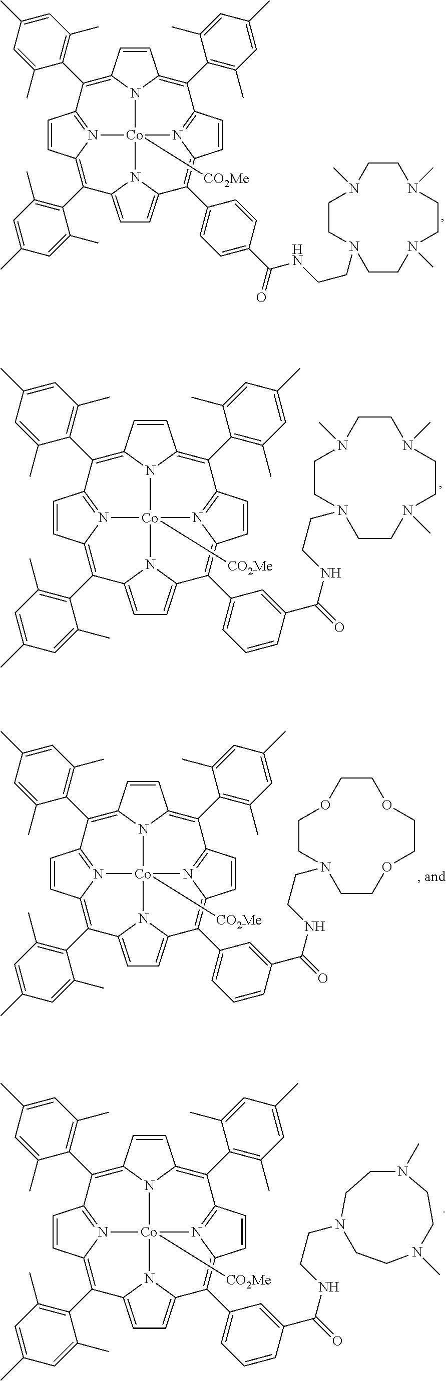

Embodiment 19 provides a method of promoting stereocontrolled living radical polymerization reaction, the method comprising:
(a) contacting the compound of any one of Embodiments 1-18 and a Lewis acid to provide a Lewis acid-catalyst complex;
(b) contacting the Lewis acid-catalyst complex with at least two vinyl monomers to provide a mixture,
   wherein each vinyl monomer is substituted with at least one substituent comprising a heteroatom having at least one coordinating lone pair of electrons.
   wherein each vinyl monomer is identical; and
(c) irradiating the mixture to provide a catalyst-nascent polymer complex.

Embodiment 20 provides the method of Embodiment 19, wherein the compound is present in an amount ranging from about 0.01 to about 20.0 mol %.

Embodiment 21 provide the method of Embodiment 19 or 20, wherein the Lewis acid comprises a rare earth metal.

Embodiment 22 provides the method of Embodiment 21, wherein the rare earth metal is La(III).

Embodiment 23 provides the method of any one of Embodiments 19-22, wherein the Lewis acid is La(OTf)₃.

Embodiment 24 provides the method of any one of Embodiments 19-23, wherein the Lewis acid is present in an amount ranging from about 0.1 to about 10.0 mol %.

Embodiment 25 provides the method of any one of Embodiments 19-24, wherein the reaction is performed in the presence of a solvent, optionally wherein the solvent is aqueous.

Embodiment 26 provides the method of Embodiment 25, wherein the solvent comprises methanol.

Embodiment 27 provides the method of any one of Embodiments 19-26, wherein the reaction is performed at about room temperature.

Embodiment 28 provides the method of any one of Embodiments Embodiment 19-27, wherein each vinyl monomer is a compound of Formula (II), which is selected from the group consisting of:

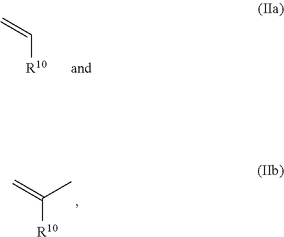

wherein:
R¹⁰ is selected from the group consisting of C(═O)N(R^{11a})(R^{11b}), C(═O)OR^{11a}, OC(═O)R^{11a}, optionally substituted C₂-C₁₀ heteroaryl. OR^{11a}, and CN;
R^{11a} and R^{11b}, if present, are independently selected from the group consisting of H, optionally substituted C₁-C₁₂ alkyl, optionally substituted C₁-C₁₂ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or
   wherein R^{11a} and R^{11b} can combine with the nitrogen atom to which they are bound to form an optionally substituted C₂-C₈ heterocycloalkyl or C₂-C₁₀ heteroaryl.

Embodiment 29 provides the method of Embodiment 28, wherein R^{11a} and R^{11b}, if present, are independently selected from the group consisting of C₁-C₆ alkyl, C₁-C₆ hydroxyalkyl, C₁-C₁₂ alkoxyalkyl, and C₁-C₆ aminoalkyl.

Embodiment 30 provides the method of Embodiment 28 or 29, wherein each occurrence of R^{11a} and R^{11b}, if present, is independently selected from the group consisting of methyl, ethyl, isopropyl. —CH₂CH₂OH, —(CH₂CH₂O)₂₋₃CH₃. —CH₂CH₂CH₂OCH₃, —CH₂CH₂CH₂N(CH₃)₂, and —CH₂CH₂CH₂N(CH₃)₃⁺.

Embodiment 31 provides the method of any one of Embodiments 19-30, wherein the vinyl monomer is selected from the group consisting of:

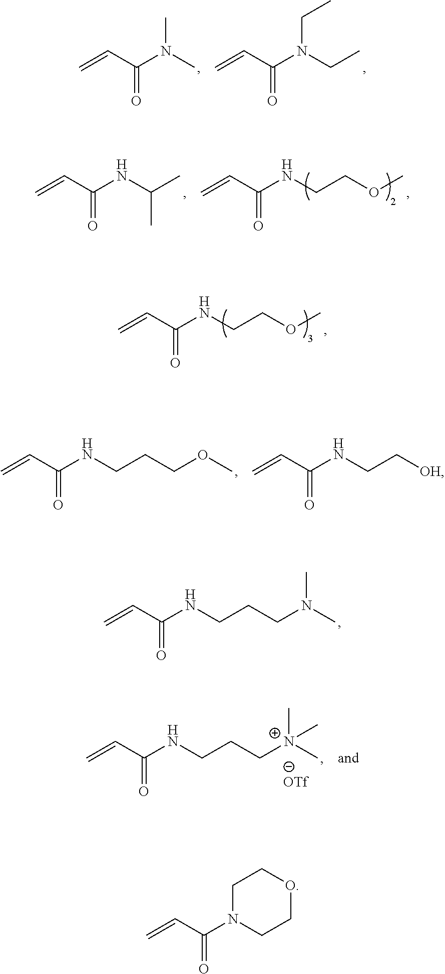

Embodiment 32 provides the method of any one of Embodiments 19-31, further comprising terminating the reaction to obtain a polymer product.

Embodiment 33 provides the method of Embodiment 32, wherein the polymer product comprises at least one selected from the group consisting of:

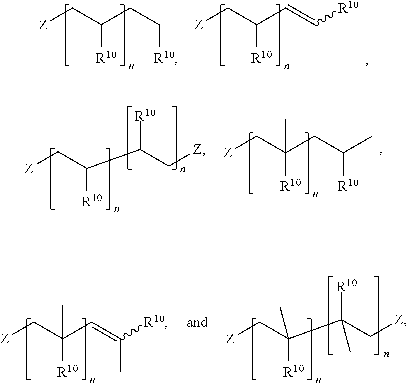

wherein:
R¹⁰ is selected from the group consisting of C(═O)N(R^{11a})(R^{11b}), C(═O)OR^{11a}, and CN;
R^{11a} and R^{11b}, if present, are independently selected from the group consisting of H, optionally substituted C₁-C₁₂ alkyl, optionally substituted C₁-C₁₂ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or
   wherein R^{11a} and R^{11b} can combine with the nitrogen atom to which they are bound to form an optionally substituted C₂-C₈ heterocycloalkyl or C₂-C₁₀ heteroaryl; and
each occurrence of n is independent an integer from 1 to 5,000.

Embodiment 34 provides the method of any one of Embodiments 28-33, wherein R¹⁰ is selected from the group consisting of:

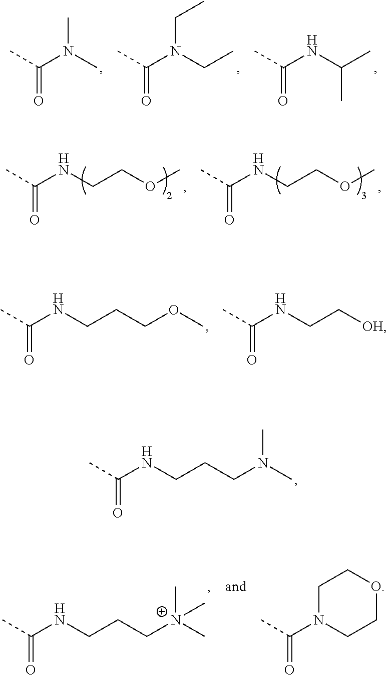

Embodiment 35 provides the method of any one of Embodiments 19-31, further comprising:
(d) contacting the catalyst-nascent polymer complex with at least two second vinyl monomers to provide a second mixture,
   wherein each second vinyl monomer is substituted with at least one substituent comprising a heteroatom having at least one coordinating lone pair of electrons,
   wherein each second vinyl monomer is substituted with identical substituents,
   wherein the first vinyl monomer and second vinyl monomer are not identical; and
(e) irradiating the second mixture to provide a second catalyst-nascent polymer complex.

Embodiment 36 provides the method of Embodiment 35, further comprising terminating the reaction to obtain a polymer product.

Embodiment 37 provides the method of any one of Embodiments 32-36, wherein the polymer product has a tacticity ranging from about 50% m to about 100% m.

Embodiment 38 provides the method of any one of Embodiments 32-37, wherein the tacticity of the polymer product is positively correlated with the amount of the Lewis acid-catalyst complex.

Embodiment 39 provides a polymer composition of Formula (III):

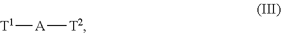

wherein:

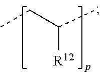

each occurrence of A comprises o units of
each occurrence of T¹ is selected from the group consisting of C(═O)OR¹³, optionally substituted alkyl, and halogen;
T² is selected from the group consisting of H,

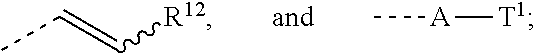

o is an integer ranging from 1 to 10;
each occurrence of p is an integer ranging from 1 to 5,000;
R¹² is substituted with at least one substituent comprising a heteroatom having at least one coordinating lone pair of electrons,
   wherein the R¹² in each unit of

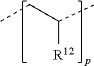

is identical; and
each occurrence of R¹³ is selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

Embodiment 40 provides the polymer of Embodiment 39, wherein T¹ is C(═O)OMe.

Embodiment 41 provides the polymer of Embodiment 39 or 40, wherein each R¹² in a unit of

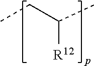

is selected from the group consisting of C(═O)N(R^{14a})(R^{13b}), C(═O)OR^{14a}, and CN;
R^{14a} and R^{14b}, if present, are independently selected from the group consisting of H, optionally substituted C₁-C₁₂ alkyl, optionally substituted C₁-C₁₂ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or
   wherein R^{14a} and R^{14b} can combine with the nitrogen atom to which they are bound to form an optionally substituted C₂-C₈ heterocycloalkyl or C₂-C₁₀ heteroaryl.

Embodiment 42 provides the polymer of any one of Embodiments 39-41, wherein R^{14a} and R^{14b} in a unit of

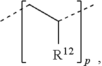

if present, are each independently selected from the group consisting of C₁-C₆ alkyl, C₁-C₆ hydroxyalkyl, C₁-C₁₂ alkoxyalkyl, and C₁-C₆ aminoalkyl.

Embodiment 43 provides the polymer of Embodiment 41 or 42, wherein R^{14a} and R^{14b} in a unit of

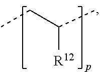

if present, are each independently selected from the group consisting of methyl, ethyl, isopropyl, —CH₂CH₂OH, —(CH₂CH₂O)₂₋₃CH₃, —CH₂CH₂CH₂OCH₃, —CH₂CH₂CH₂N(CH₃)₂, and —CH₂CH₂CH₂N(CH₃)₃*.

Embodiment 44 provides the polymer of any one of Embodiments 39-43, wherein R¹² in a unit of

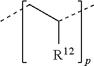

is selected from the group consisting of:

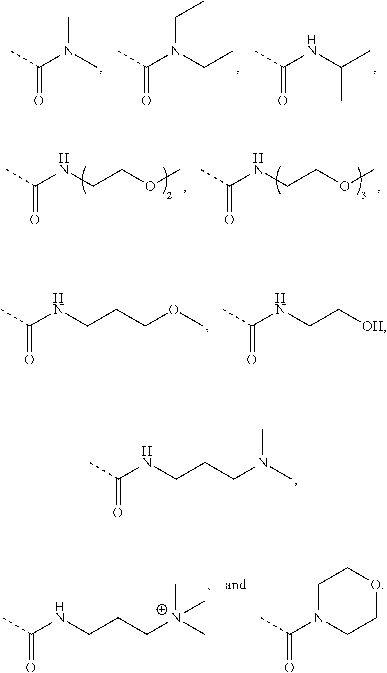

Embodiment 45 provides the polymer of any one of Embodiments 39-44, wherein the polymer has a tacticity ranging from about 50% m to about 100% m.

Embodiment 46 provides the polymer of any one of Embodiments 39-45, wherein the polymer is prepared according to any one of Embodiments 19-38.

Embodiment 47 provides a pharmaceutical composition comprising at least one therapeutic agent at least partially encapsulated in the polymer of any one of Embodiments 39-46.

Embodiment 48 provides the pharmaceutical composition of Embodiment 47, wherein the polymer comprises at least one selected from the group consisting of polyDEAA and polyNIPAM.

Embodiment 49 provides an adhesive composition comprising the polymer of any one of Embodiments 39-46.

Embodiment 50 provides the adhesive composition of Embodiment 49, wherein the polymer comprises polyHEAA.

Embodiment 51 provides an ion-exchange membrane composition comprising the polymer of any one of Embodiments 39-46, wherein the polymer is substituted with at least one ionic substituent.

Embodiment 52 provides the ion-exchange membrane composition of Embodiment 51, wherein the polymer comprises polyAPTMAT.

Embodiment 53 provides a battery comprising the ion-exchange membrane composition of Embodiment 51 or 52.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound selected from the group consisting of: 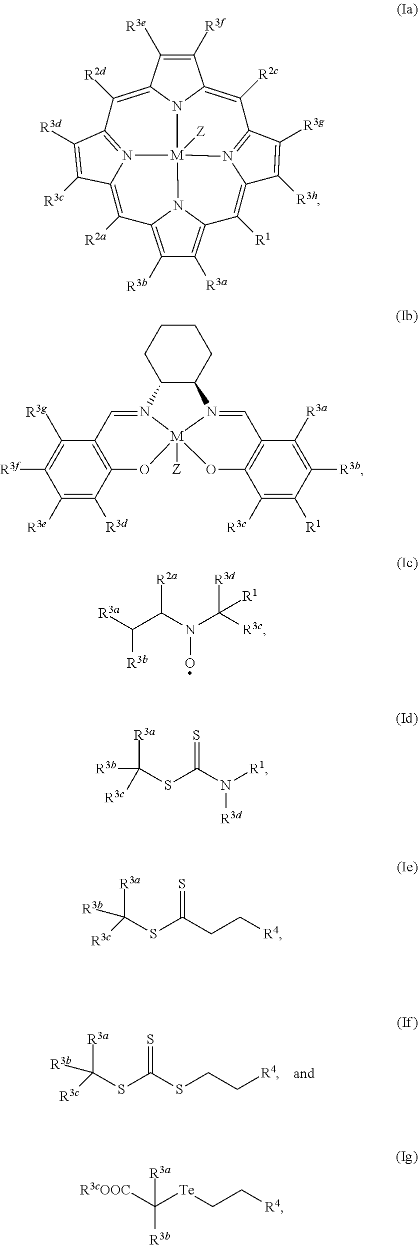
or a salt, solvate, stereoisomer, or isotopologue thereof, wherein:
M, if present, is Co;
Z is absent or an anionic ligand;
R¹, if present, is 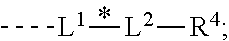
R^{2a}, R^{2b}, and R^{2c}, if present, are each independently selected from the group consisting of H, optionally substituted C₁-C₆ alkyl, optionally substituted C₃-C₈ cycloalkyl, optionally substituted C₆-C₁₀ aryl and optionally substituted C₂-C₁₀ heteroaryl;
R^{3a}, R^{3b}, R^{3c}, R^{3d}, R^{3e}, R^{3f}, R^{3g}, and R^{3h}, if present, are each independently selected from the group consisting of H and optionally substituted C₁-C₆ alkyl;
L¹, if present, is selected from the group consisting of -(optionally substituted C₆-C₁₀ arylenyl)-* and -(optionally substituted C₂-C₁₀ heteroarylenyl)-*,
wherein a substituent of the C₆-C₁₀ arylenyl or the C₂-C₁₀ heteroarylenyl in L¹ can combine with a substituent of the C₆-C₁₀ aryl or C₂-C₁₀ heteroaryl in R^{2b} to form an optionally substituted C₂₀-C₃₀ heterocycloalkenyl;
L², if present, is selected from the group consisting of *—C(═O)N(R⁵)(optionally substituted C₁-C₆ alkylenyl)-, *—C(═O)O(optionally substituted C₁-C₆ alkylenyl)-, *—C(═O)(optionally substituted C₁-C₆ alkylenyl)-, *-(optionally substituted C₁-C₆ alkylenyl)-, *—N(R⁵)C(═O)(optionally substituted C₁-C₆ alkylenyl)-, *—OC(═O)(optionally substituted C₁-C₆ alkylenyl)-, *—C(═O)N(R⁵)(optionally substituted C₁-C₆ heteroalkylenyl)-, *—C(═O)O(optionally substituted C₁-C₆ heteroalkylenyl)-, *—C(═O)(optionally substituted C₁-C₆ heteroalkylenyl)-, *-(optionally substituted C₁-C₆ heteroalkylenyl)-, *—N(R⁵)C(═O)(optionally substituted C₁-C₆ heteroalkylenyl)-, and *—OC(═O)(optionally substituted C₁-C₆ heteroalkylenyl)-;
R⁴ is an optionally substituted C₆-C₈ heterocycloalkyl,
wherein the C₆-C₈ heterocycloalkyl comprises at least three atoms selected from the group consisting of N, O, and S; and
each occurrence of R⁵, if present, is independently selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

2. The compound of claim 1, wherein at least one of the following applies:
(a) L¹ is selected from the group consisting of: 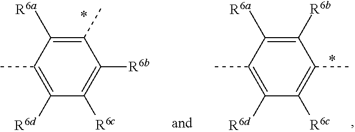
wherein R^{6a}, R^{6b}, R^{6c}, and R^{6d} are each independently selected from the group consisting of H, optionally substituted C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵;
(b) L² is *—C(═O)NH(C₁-C₆ alkylenyl)-, optionally wherein L² is *—C(═O)NHCH₂CH₂—,
(c) R⁴ is selected from the group consisting of: 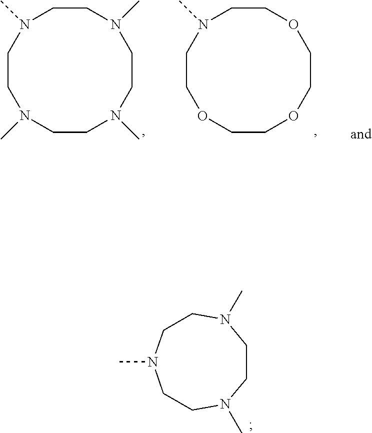
(d) R^{2a}, R^{2b}, and R^{2c} are each independently 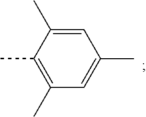
and
(e) Z is absent or selected from the group consisting of C(═O)OR⁵, optionally substituted alkyl, and halogen, optionally wherein C(═O)OR⁵ is C(═O)OMe.

3-5. (canceled)

6. The compound of claim 1, wherein R⁴ is: 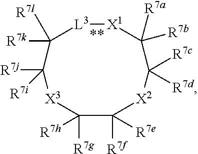
wherein:
L³ is selected from the group consisting of a bond and —X⁴—C(R^{7m})(R⁷ⁿ)—C(R^{7o})(R^{7p})—**;
X¹, X², X³, and X⁴, if present, are each independently selected from the group consisting of N(R⁸) and O;
R^{7a}, R^{7b}, R^{7c}, R^{7d}, R^{7e}, R^{7f}, R^{7g}, R^{7h}, R⁷ⁱ, R^{7j}, R^{7k}, R^{7l}, R^{7m}, R⁷ⁿ, R^{7o}, and R^{7p}, if present, are each independently selected from the group consisting of H, C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵,
wherein one of R^{7a}, R^{7b}, R^{7c}, R^{7d}, R^{7e}, R^{7f}, R^{7g}, R^{7h}, R⁷ⁱ, R^{7j}, R^{7k}, and R^{7l} is -L²-*, or
one of X¹, X², and X³ is N-L²-*; and
R⁸ is selected from the group consisting of H and optionally substituted C₁-C₆ alkyl.

7-9. (canceled)

10. The compound of claim 1, wherein R^{2a}, R^{2b}, and R^{2d} are each independently: 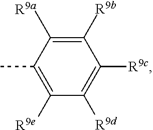
wherein R^{9a}, R^{9b}, R^{9c}, R^{9d}, and R^{9e} are each independently selected from the group consisting of H, optionally substituted C₁-C₆ alkyl, CN, NO₂, OR⁵, N(R⁵)(R⁵), halogen, C(═O)OR⁵, C(═O)N(R⁵)(R⁵), C(═O)R⁵, OC(═O)R⁵, and N(R⁵)C(═O)R⁵,
optionally wherein R^{9a}, R^{9b}, R^{9c}, R^{9d}, and R^{9e} are each independently selected from the group consisting of H and CH₃.

11-14. (canceled)

15. The compound of claim 1, which is selected from the group consisting of:

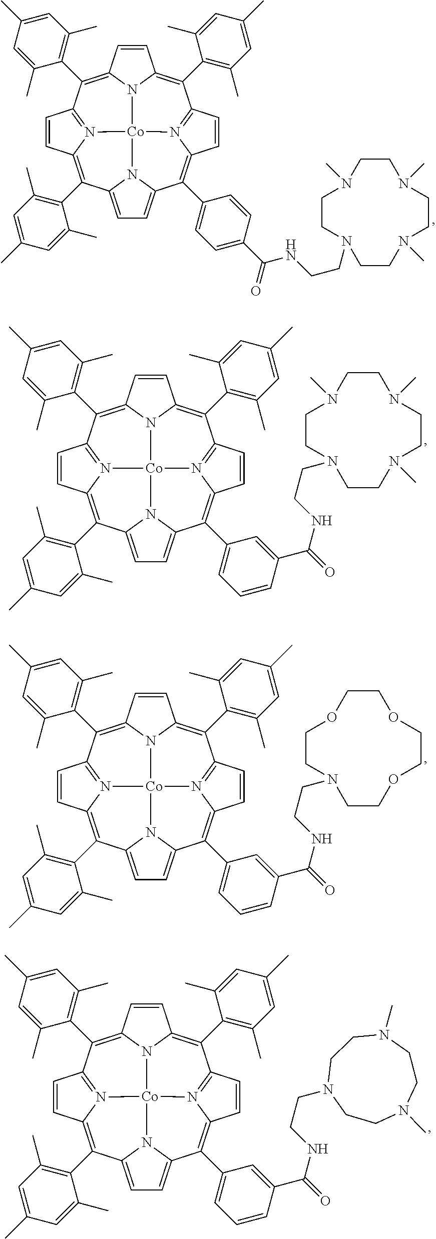

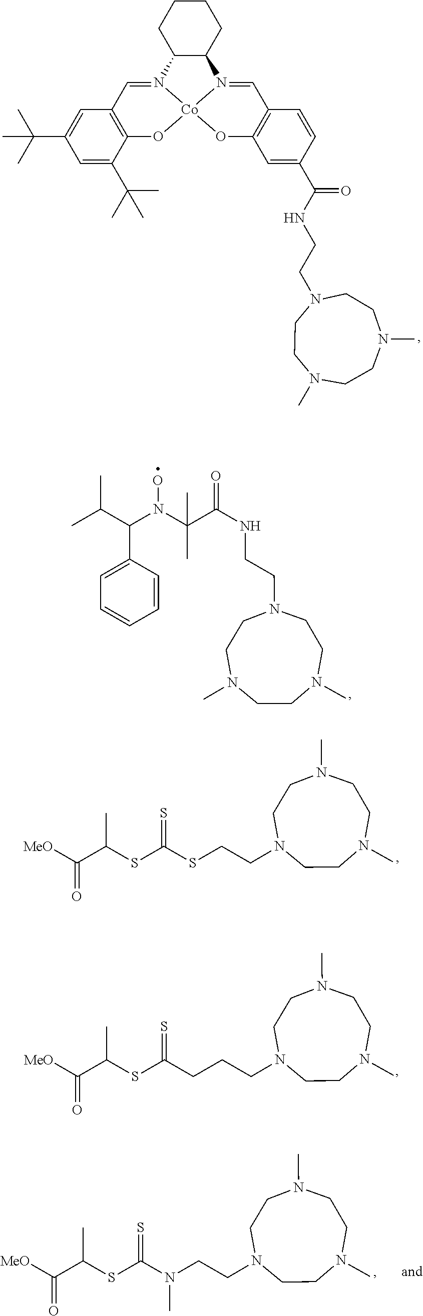

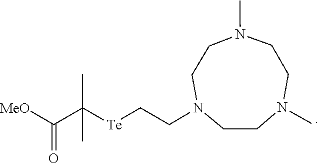

16-17. (canceled)

18. The compound of claim 1, which is selected from the group consisting of:

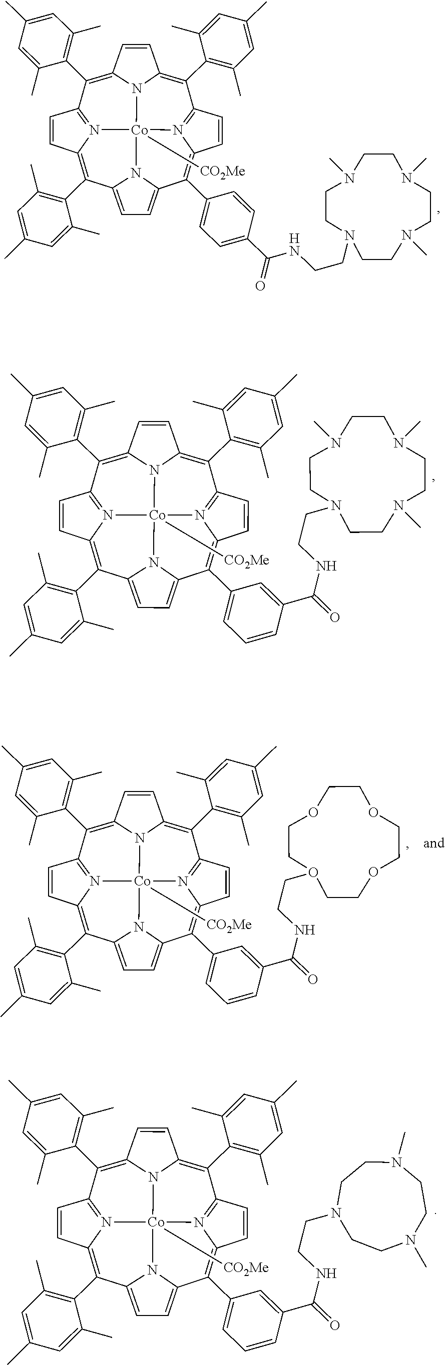

19. A method of promoting stereocontrolled living radical polymerization reaction, the method comprising:
(a) contacting the compound of claim 1 and a Lewis acid to provide a Lewis acid-catalyst complex;
(b) contacting the Lewis acid-catalyst complex with at least two vinyl monomers to provide a mixture,
wherein each vinyl monomer is substituted with at least one substituent comprising a heteroatom having at least one coordinating lone pair of electrons,
wherein each vinyl monomer is identical; and
(c) irradiating the mixture to provide a catalyst-nascent polymer complex.

20. The method of claim 19, wherein at least one of the following applies:
(a) the compound is present in an amount ranging from about 0.01 to about 20.0 mol %;
(b) the Lewis acid comprises a rare earth metal, optionally wherein the rare earth metal is La(III), and optionally wherein the Lewis acid is La(OTf)₃;
(c) the Lewis acid is present in an amount ranging from about 0.1 to about 10.0 mol %;
(d) the reaction is performed in the presence of a solvent, optionally wherein the solvent is aqueous, and optionally wherein the solvent comprises methanol; and
(e) the reaction is performed at about room temperature.

21-27. (canceled)

28. The method of claim 19, wherein each vinyl monomer is a compound of Formula (II), which is selected from the group consisting of: 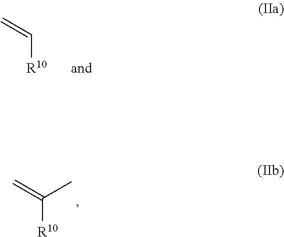
wherein:
R¹⁰ is selected from the group consisting of C(═O)N(R^{11a})(R^{11b}), C(═O)OR^{11a}, OC(═O)R^{11a}, optionally substituted C₂-C₁₀ heteroaryl, OR^{11a}, and CN;
R^{11a} and R^{11b}, if present, are independently selected from the group consisting of H, optionally substituted C₁-C₁₂ alkyl, optionally substituted C₁-C₁₂ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or
wherein R^{11a} and R^{11b} can combine with the nitrogen atom to which they are bound to form an optionally substituted C₂-C₈ heterocycloalkyl or C₂-C₁₀ heteroaryl.

29. The method of claim 28, wherein at least one of the following applies:
(a) R^{11a} and R^{11b}, if present, are independently selected from the group consisting of C₁-C₆ alkyl, C₁-C₆ hydroxyalkyl, C₁-C₁₂ alkoxyalkyl, and C₁-C₆ aminoalkyl, optionally wherein each occurrence of R^{11a} and R^{11b}, if present, is independently selected from the group consisting of methyl, ethyl, isopropyl, —CH₂CH₂OH, —(CH₂CH₂O)₂₋₃CH₃, —CH₂CH₂CH₂OCH₃, —CH₂CH₂CH₂N(CH₃)₂, and —CH₂CH₂CH₂N(CH₃)₃⁺; and
(b) R¹⁰ is selected from the group consisting of

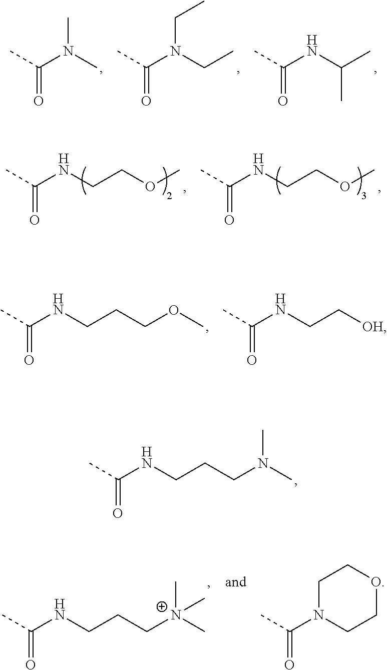

30. (canceled)

31. The method of claim 19, wherein the vinyl monomer is selected from the group consisting of:

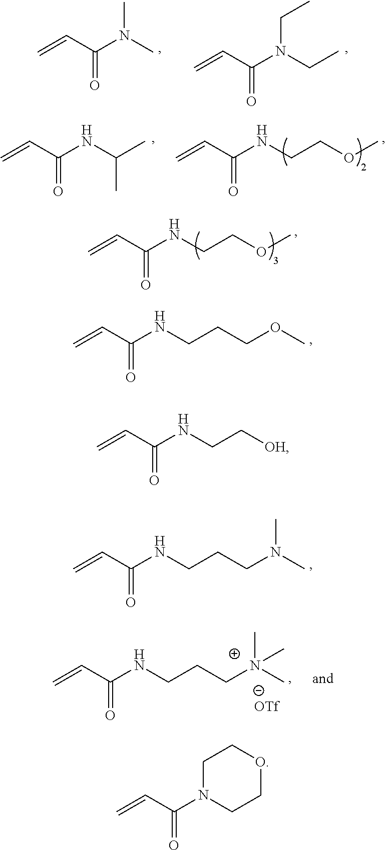

32. The method of claim 19, further comprising terminating the reaction to obtain a polymer product,
optionally wherein the polymer product comprises at least one selected from the group consisting of: 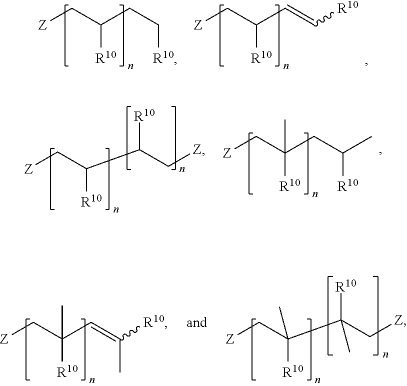
wherein:
R¹⁰ is selected from the group consisting of C(═O)N(R^{11a})(R^{11b}), C(═O)OR^{11a}, and CN;
R^{11a} and R^{11b}, if present, are independently selected from the group consisting of H, optionally substituted C₁-C₁₂ alkyl, optionally substituted C₁-C₁₂ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or
wherein R^{11a} and R^{11b} can combine with the nitrogen atom to which they are bound to form an optionally substituted C₂-C₈ heterocycloalkyl or C₂-C₁₀ heteroaryl; and
each occurrence of n is independent an integer from 1 to 5,000.

33-34. (canceled)

35. The method of claim 19, further comprising:
(d) contacting the catalyst-nascent polymer complex with at least two second vinyl monomers to provide a second mixture,
wherein each second vinyl monomer is substituted with at least one substituent comprising a heteroatom having at least one coordinating lone pair of electrons,
wherein each second vinyl monomer is substituted with identical substituents,
wherein the first vinyl monomer and second vinyl monomer are not identical;
(e) irradiating the second mixture to provide a second catalyst-nascent polymer complex; and
(f) optionally terminating the reaction to obtain a polymer product.

36-38. (canceled)

39. A polymer composition of Formula (III): 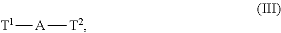
wherein:
each occurrence of A comprises o units of 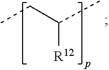
each occurrence of T¹ is selected from the group consisting of C(═O)OR¹³, optionally substituted alkyl, and halogen;
T² is selected from the group consisting of H, 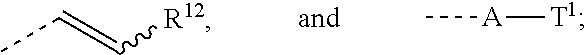
o is an integer ranging from 1 to 10;
each occurrence of p is an integer ranging from 1 to 5,000;
R¹² is substituted with at least one substituent comprising a heteroatom having at least one coordinating lone pair of electrons,
wherein the R¹² in each unit of 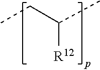
is identical; and
each occurrence of R¹³ is selected from the group consisting of H, optionally substituted C₁-C₆ alkyl, optionally substituted C₃-C₈ cycloalkyl, optionally substituted C₂-C₁₀ heterocyclyl, and optionally substituted C₆-C₁₀ aryl.

40. The polymer of claim 39, wherein at least one of the following applies:
(a) T¹ is C(═O)OMe; and
(b) each R¹² in a unit of 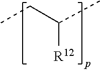
is selected from the group consisting of C(═O)N(R^{14a})(R^{13b}), C(═O)OR^{14a}, and CN;
R^{14a} and R^{14b}, if present, are independently selected from the group consisting of H, optionally substituted C₁-C₁₂ alkyl, optionally substituted C₁-C₁₂ heteroalkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl, or
wherein R^{14a} and R^{14b} can combine with the nitrogen atom to which they are bound to form an optionally substituted C₂-C₈ heterocycloalkyl or C₂-C₁₀ heteroaryl.

41. (canceled)

42. The polymer of claim 39, wherein at least one of the following applies:
(a) R^{4a} and R^{14b} in a unit of 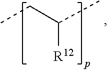
if present, are each independently selected from the group consisting of C₁-C₆ alkyl, C₁-C₆ hydroxyalkyl, C₁-C₁₂ alkoxyalkyl, and C₁-C₆ aminoalkyl, optionally wherein R^{14a} and R^{14b} in a unit of 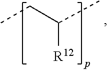
if present, are each independently selected from the group consisting of methyl, ethyl, isopropyl, —CH₂CH₂OH, —(CH₂CH₂O)₂₋₃CH₃, —CH₂CH₂CH₂OCH₃, —CH₂CH₂CH₂N(CH₃)₂, and —CH₂CH₂CH₂N(CH₃)₃⁺; and
(b) R¹² in a unit of 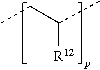
is selected from the group consisting of:

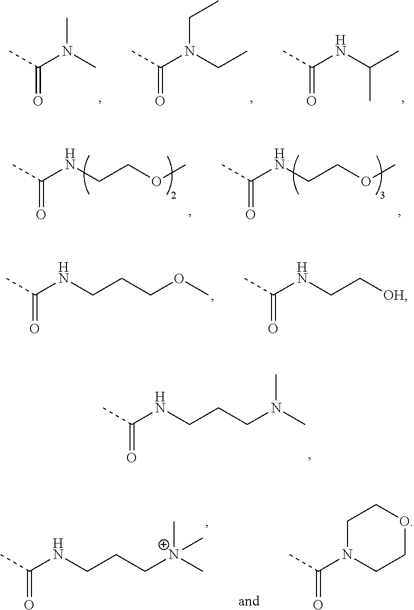

43-46. (canceled)

47. A pharmaceutical composition comprising at least one therapeutic agent at least partially encapsulated in the polymer of claim 39,
optionally wherein the polymer comprises at least one selected from the group consisting of polyDEAA and polyNIPAM.

48. (canceled)

49. An adhesive composition comprising the polymer of claim 39,
optionally wherein the polymer comprises polyHEAA.

50. (canceled)

51. An ion-exchange membrane composition comprising the polymer of claim 39, wherein the polymer is substituted with at least one ionic substituent,
optionally wherein the polymer comprises polyAPTMAT.

52. (canceled)

53. A battery comprising the ion-exchange membrane composition of claim 51.
